(19) United States
(12) Patent Application Publication
(10) Pub. No.: US 2024/0292739 A1
(43) Pub. Date: Aug. 29, 2024

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Hyun KIM, Gyeonggi-do (KR); Hyun-Woo KANG, Gyeonggi-do (KR); Hong-Se OH, Gyeonggi-do (KR); So-Mi PARK, Gyeonggi-do (KR); Jin-Man KIM, Gyeonggi-do (KR); Dong-Hyung LEE, Gyeonggi-do (KR); Samuel KIM, Gyeonggi-do (KR); Tae-Jin LEE, Gyeonggi-do (KR)

(21) Appl. No.: 18/417,102

(22) Filed: Jan. 19, 2024

(30) Foreign Application Priority Data
Feb. 2, 2023 (KR) 10-2023-0014289
Jan. 3, 2024 (KR) 10-2024-0000823

(51) Int. Cl. H10K 85/60; C07C 211/61; C07D 307/91; C07D 333/76; C09K 11/06; H10K 50/11; H10K 50/15; H10K 50/18

(52) U.S. Cl. CPC H10K 85/633; C07C 211/61; C07D 307/91; C07D 333/76; C09K 11/06; H10K 85/636; C07C 2603/40; C07C 2603/86; C09K 2211/1003; C09K 2211/1018; H10K 50/11; H10K 50/156; H10K 50/181; H10K 85/622; H10K 85/626; H10K 85/6574; H10K 85/6576

(57) ABSTRACT

The present disclosure relates to an organic electroluminescent compound represented by formula 1 and an organic electroluminescent device comprising the same. By comprising an organic electroluminescent compound according to the present disclosure, it is possible to produce an organic electroluminescent device having improved current efficiency and/or lifetime properties.

### TECHNICAL FIELD

The present disclosure relates to an organic electroluminescent compound, and an organic electroluminescent device comprising the same.

### BACKGROUND ART

An electroluminescent device (EL device) is a self-luminous display device that has the advantages of a wide viewing angle, excellent contrast, and fast response speed. In 1987, Eastman Kodak first developed an organic electroluminescent device using small aromatic diamine molecules and aluminum complexes as materials for forming a light-emitting layer [Appl. Phys. Lett. 51, 913, 1987].

An organic electroluminescent device has a multi-layer structure including a hole injection layer, a hole transport layer, a light-emitting layer, an electron transport layer, and an electron injection layer, etc., to increase efficiency and stability. At this time, selection of compounds included in the hole transport layer is recognized as one of the means to improve device characteristics such as hole transport efficiency to the light-emitting layer, luminous efficiency, and life time.

In this regard, copper phthalocyanine (CuPc), 4,4′-bis[N-(1-naphthyl)-N-phenylamino]biphenyl (NPB), N,N′-diphenyl-N,N′-bis(3-methylphenyl)-(1,1′-biphenyl)-4,4′-diamine (TPD), 4,4′,4″-tris(3-methylphenylphenylamino)triphenylamine (MTDATA), etc. have been used as hole injection and transport materials in an organic electroluminescent device. However, when these materials are used, the quantum efficiency and lifetime of an organic electroluminescent device are reduced. The reason is that, when an organic electroluminescent device is driven at a high current, thermal stress occurs between the anode and the hole injection layer, and the lifetime of the device is rapidly reduced due to this thermal stress. In addition, since the organic material used in the hole injection layer has very high hole mobility, the hole-electron charge balance is broken, resulting in a decrease in quantum efficiency (cd/A).

Meanwhile, Korean Patent Application Laying-Open No. 2017-0124957 discloses an organic electroluminescent device comprising a benzofluorene compound comprising an amino group. However, the aforementioned reference does not specifically disclose a compound claimed herein, and the development of hole transport materials to improve OLED performance is still required.

### DISCLOSURE OF INVENTION

### Technical Problem

The objective of the present disclosure is firstly, to provide an organic electroluminescent compound effective in producing an organic electroluminescent device with improved current efficiency and/or lifetime characteristics. Secondly, the objective of the present disclosure is to provide an organic electroluminescent device comprising the organic electroluminescent compound.

### Solution to Problem

As a result of intensive studies to solve the technical problems, the present inventors found that the above objective can be achieved by an organic electroluminescent compound represented by the following formula 1.

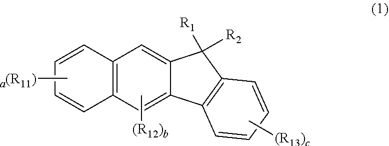

In formula 1,
R₁ and R₂, each independently, represent a substituted or unsubstituted (C1-C30)alkyl, a substituted or unsubstituted (C6-C30)aryl, or a substituted or unsubstituted (3- to 30-membered)heteroaryl; or may be linked to each other to form a ring(s);
R₁₁ to R₁₃, each independently, represent hydrogen, deuterium, or a substituted or unsubstituted (C6-C30)aryl, or are represented by the following formula 1-1, with the proviso that at least one of R₁₂ and R₁₃ is represented by the following formula 1-1;

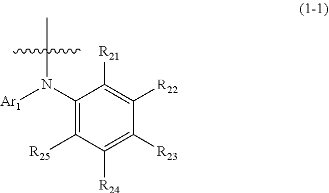

in formula 1-1,
Ar₁ represents a substituted or unsubstituted (C6-C30)aryl, or a substituted or unsubstituted (3- to 30-membered)heteroaryl;
R₂₁ and R₂₅, each independently, represent hydrogen, deuterium, an unsubstituted (C1-C30)alkyl, or a combination thereof;
R₂₂ to R₂₄, each independently, represent hydrogen, deuterium, a substituted or unsubstituted (C1-C30)alkyl, a substituted or unsubstituted (C6-C30)aryl, or a substituted or unsubstituted (3- to 30-membered)heteroaryl, with the proviso that at least one of R₂₂ to R₂₄ represents a substituted or unsubstituted (C12-C30)aryl comprising the following linking group a in ortho form;

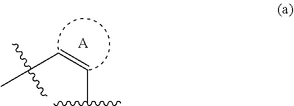

ring A represents a substituted or unsubstituted (C6-C24)arene ring;
a and c, each independently, represent an integer of 1 to 4, and b represents an integer of 1 to 2, in which if a to c represent an integer of 2 or more, each of R₁ to each of R₁₃ may be the same or different; and
the heteroaryl and heteroarene each comprise at least one heteroatom selected from B, N, O, S, Si and P.

### Advantageous Effects of Invention

By using the organic electroluminescent compound according to the present disclosure, an organic electroluminescent device with improved current efficiency and/or lifetime characteristics can be provided.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc. The hole transport zone material may be at least one selected from the group consisting of hole transport materials, hole injection materials, electron blocking materials, hole auxiliary materials, and light-emitting auxiliary materials.

The organic electroluminescent material of the present disclosure may comprise at least one compound represented by formula 1 above. The compound of formula 1 may be comprised in at least one layer constituting an organic electroluminescent device, and may be comprised in at least one layer among the layers constituting a hole transport zone, but is not limited thereto. When the compound of formula 1 may be comprised in a hole transport layer, a hole auxiliary layer, an electron blocking layer, a light-emitting layer, or a light-emitting auxiliary layer, it may be included as a hole transport material, a hole auxiliary material, an electron blocking material, a host material, or a light-emitting auxiliary material.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, and 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7 ring backbone atoms, preferably having 5 to 7, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” or “(C6-C30)arene” is meant to be a monocyclic or fused ring radical derived from an aromatic hydrocarbon having 6 to 30 ring backbone carbon atoms. The above aryl may be partially saturated, and may comprise a spiro structure. The number of the ring backbone carbon atoms is preferably 6 to 25, more preferably 6 to 18. The above aryl may include phenyl, biphenyl, terphenyl, naphthyl, binaphthyl, phenylnaphthyl, naphthylphenyl, phenylterphenyl, fluorenyl, phenylfluorenyl, diphenylfluorenyl, dimethylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” and “(3- to 30-membered)heteroarene” are meant to be an aryl(ene) group having 3 to 30 ring backbone atoms, and including at least one, preferably 1 to 4 heteroatoms selected from the group consisting of B, N, O, S, Si, P, Se, Te, and Ge. The above heteroaryl(ene) may be a monocyclic ring, or a fused ring condensed with at least one benzene ring; may be partially saturated; may be one formed by linking at least one heteroaryl or aryl group to a heteroaryl(ene) group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, benzophenanthrofuranyl, dibenzothiophenyl, benzimidazolyl, benzothiazolyl, benzoisothiazolyl, benzophenanthrothiophenyl, benzoisoxazolyl, benzoxazolyl, phenanthrooxazolyl, phenanthrothiazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. “Heteroaryl(ene)” can be classified into heteroaryl(ene) with electronic properties and heteroaryl(ene) with hole properties. Heteroaryl(ene) with electronic properties is a substituent which is relatively rich in electrons in the parent nucleus, for example, a substituted or unsubstituted pyridyl, a substituted or unsubstituted pyrimidinyl, a substituted or unsubstituted triazinyl, a substituted or unsubstituted quinazolinyl, a substituted or unsubstituted quinoxalinyl, or a substituted or unsubstituted quinolyl, etc. Heteroaryl(ene) with hole properties is a substituent which is relatively poor in electrons in the parent nucleus, for example, a substituted or unsubstituted carbazolyl, a substituted or unsubstituted dibenzofuranyl, a substituted or unsubstituted dibenzothiophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2 or positions 2 and 3, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Unless otherwise specified, the substituent may replace hydrogen at a position where the substituent can be substituted without limitation, and when two or more hydrogen atoms in a certain functional group are each replaced with a substituent, each substituent may be the same or different from each other. The maximum number of substituents that can be substituted for a certain functional group may be the total number of valences that can be substituted for each atom forming the functional group. Herein, the substituted aryl, the substituted arene, the substituted heteroaryl, the substituted heteroarene, and the substituted alkyl, each independently, are substituted with at least one selected from the group consisting of deuterium, a halogen, a cyano, a carboxyl, a nitro, a hydroxy, a phosphine oxide, a (C1-C30)alkyl unsubstituted or substituted with deuterium, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (3- to 30-membered)heteroaryl unsubstituted or substituted with a (C6-C30)aryl(s), a (C6-C30)aryl unsubstituted or substituted with a (3- to 30-membered)heteroaryl(s), a tri(C1-C30)alkylsilyl, a tri(C6-C30)arylsilyl, a di(C1-C30)alkyl(C6-C30)arylsilyl, a (C1-C30)alkyldi(C6-C30)arylsilyl, a fused ring group of a (C3-C30)aliphatic ring(s) and a (C6-C30)aromatic ring(s), an amino, a mono- or di-(C1-C30)alkylamino, a mono- or di-(C2-C30)alkenylamino, a mono- or di-(C6-C30)arylamino, a mono- or di-(3- to 30-membered)heteroarylamino, a (C1-C30)alkyl(C2-C30)alkenylamino, a (C1-C30)alkyl(C6-C30)arylamino, a (C1-C30)alkyl(3- to 30-membered)heteroarylamino, a (C2-C30)alkenyl(C6-C30)arylamino, a (C2-C30)alkenyl(3- to 30-membered)heteroarylamino, a (C6-C30)aryl(3- to 30-membered)heteroarylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl. According to one embodiment of the present disclosure, the substituted aryl, etc., each independently, are substituted with at least one selected from the group consisting of a (C1-C30)alkyl unsubstituted or substituted with deuterium, and a (C6-C30)aryl. According to another embodiment of the present disclosure, the substituted aryl, etc., each independently, are substituted by at least one selected from the group consisting of deuterium, a (C1-C10)alkyl unsubstituted or substituted with deuterium, and a (C6-C20)aryl. For example, the substituted aryl, etc., each independently, may be substituted with at least one selected from the group consisting of deuterium, a methyl, a tert-butyl, an isopropyl unsubstituted or substituted with deuterium, a methyl substituted with deuterium, a phenyl, a naphthyl, a cyclohexyl, a bicyclo(2,2,1)heptyl, or an adamantyl.

If a substituent is not indicated in the chemical formulas or compound structures herein, it may mean that all positions at which may be substituted are hydrogen or deuterium. That is, in the case of deuterium, it is an isotope of hydrogen, and some hydrogen atoms may be the isotope deuterium, and in this case, the content of deuterium may be 0% to 100%. In cases where substituents are not indicated in the chemical formulas or compound structures herein, if deuterium is not explicitly excluded, for example unless the content of deuterium is 0% and the content of hydrogen is 100%, unless all substituents are hydrogen, hydrogen and deuterium can be used together in the compound. The deuterium is one of the isotopes of hydrogen and is an element that has a deuteron consisting of one proton and one neutron, as its nucleus. The deuterium may be expressed as hydrogen-2, and may be written as element symbol D or 2H. The isotopes refer to atoms with the same atomic number (Z) but different mass numbers (A), and may also be interpreted as elements with the same number of protons but different numbers of neutrons.

The term “a combination thereof” in the present disclosure refers to a combination of one or more elements from the concerned list to form a known or chemically stable arrangement that may be envisioned by one skilled in the art from the concerned list. For example, alkyl and deuterium can be combined to form a partially or fully deuterated alkyl group; halogen and alkyl can be combined to form a halogenated alkyl substituent; halogen, alkyl, and aryl can be combined to form a halogenated arylalkyl. For example, preferred combinations of substituents include up to 50 atoms that are not hydrogen or deuterium, or up to 40 atoms that are not hydrogen or deuterium, or up to 30 atoms that are not hydrogen or deuterium. Alternatively, in many cases, preferred combinations of substituents may include up to 20 atoms that are not hydrogen or deuterium.

Hereinafter, the compound represented by formula 1 will be described in more detail.

In formula 1, R₁ and R₂, each independently, represent a substituted or unsubstituted (C1-C30)alkyl, a substituted or unsubstituted (C6-C30)aryl, or a substituted or unsubstituted (3- to 30-membered)heteroaryl; or may be linked to each other to form a ring(s). According to one embodiment of the present disclosure, R₁ and R₂, each independently, represent a substituted or unsubstituted (C1-C10)alkyl, or a substituted or unsubstituted (C6-C20)aryl. According to another embodiment of the present disclosure, R₁ and R₂, each independently, represent an unsubstituted (C1-C10)alkyl, or an unsubstituted (C6-C20)aryl. According to another embodiment of the present disclosure, R₁ and R₂, each independently, represent a methyl unsubstituted or substituted with deuterium, an ethyl unsubstituted or substituted with deuterium, or a phenyl unsubstituted or substituted with deuterium. For example, R₁ and R₂, each independently, may be a methyl, an ethyl, a phenyl, etc.

In formula 1, R₁₁ to R₁₃, each independently, represent hydrogen, deuterium, or a substituted or unsubstituted (C6-C30)aryl, or are represented by formula 1-1, with the proviso that at least one of R₁₂ and R₁₃ is represented by formula 1-1. According to one embodiment of the present disclosure, R₁₁ to R₁₃ each independently, represent hydrogen, or a substituted or unsubstituted (C6-C20)aryl, or are represented by formula 1-1. According to another embodiment of the present disclosure, R₁₁ to R₁₃, each independently, represent hydrogen, or an unsubstituted (C6-C20)aryl, or are represented by formula 1-1. According to another embodiment of the present disclosure, R₁₁ to R₁₃, each independently, represent hydrogen, a methyl unsubstituted or substituted with deuterium, or a phenyl unsubstituted or substituted with deuterium, or are represented by formula 1-1. For example, R₁₁ to R₁₃, each independently, may be hydrogen, a phenyl, etc., or may be represented by formula 1-1.

In formula 1-1, Ar₁ represents a substituted or unsubstituted (C6-C30)aryl, or a substituted or unsubstituted (3- to 30-membered)heteroaryl. According to one embodiment of the present disclosure, Ar₁ represents a substituted or unsubstituted (C6-C20)aryl, or a substituted or unsubstituted (5- to 20-membered)heteroaryl. According to another embodiment of the present disclosure, Ar₁ represents a (C6-C20)aryl unsubstituted or substituted with at least one of deuterium and a (C1-C10)alkyl(s) unsubstituted or substituted with deuterium; or an unsubstituted (5- to 20-membered)heteroaryl. For example, Ar₁ may be a phenyl unsubstituted or substituted with a methyl(s), an isopropyl(s) unsubstituted or substituted with deuterium, a methyl(s) substituted with deuterium, a cyclohexyl(s), a bicyclo(2,2,1)heptyl(s), or an adamantyl(s); a naphthyl; a biphenyl unsubstituted or substituted with a methyl(s), a tert-butyl(s), or a methyl(s) substituted with deuterium; dimethylfluorenyl; terphenyl; dibenzofuranyl; dibenzothiophenyl, etc.

In formula 1-1, R₂₁ and R₂₅, each independently, represent hydrogen, deuterium, an unsubstituted (C1-C30)alkyl, or a combination thereof. According to one embodiment of the present disclosure, R₂₁ and R₂₅, each independently, represent hydrogen, or a substituted or unsubstituted (C1-C10)alkyl. According to another embodiment of the present disclosure, R₂₁ and R₂₅, each independently, represent hydrogen, or a (C1-C10)alkyl unsubstituted or substituted with deuterium. For example, R₂₁ and R₂₅, each independently, may be hydrogen, a methyl, a methyl substituted with deuterium, etc.

In formula 1-1, R₂₂ to R₂₄, each independently, represent hydrogen, deuterium, a substituted or unsubstituted (C1-C30)alkyl, a substituted or unsubstituted (C6-C30)aryl, or a substituted or unsubstituted (3- to 30-membered)heteroaryl, with the proviso that at least one of R₂₂ to R₂₄ represents a substituted or unsubstituted (C12-C30)aryl comprising linking group a in ortho form. According to one embodiment of the present disclosure, R₂₂ to R₂₄, each independently, represent hydrogen, a substituted or unsubstituted (C1-C10)alkyl, or a substituted or unsubstituted (C12-C20)aryl comprising linking group a in ortho form. According to another embodiment of the present disclosure, R₂₂ to R₂₄, each independently, represent hydrogen, an unsubstituted (C1-C10)alkyl, or an unsubstituted (C12-C20)aryl comprising linking group a in ortho form. For example, R₂₂ to R₂₄, each independently, may be hydrogen, a methyl, etc.; or a biphenyl, a phenylnaphthyl, a naphthylphenyl, a terphenyl, etc., comprising linking group a in ortho form.

In linking group a, ring A represents a substituted or unsubstituted (C6-C24)arene ring. According to one embodiment of the present disclosure, ring A represents a substituted or unsubstituted (C6-C12)arene ring. According to another embodiment of the present disclosure, ring A represents an unsubstituted (C6-C12)arene ring. For example, ring A may be a benzene ring, a naphthalene ring, etc.

In formula 1, a and c, each independently, represent an integer of 1 to 4, and b represents an integer of 1 to 2, in which if a to c represent an integer of 2 or more, each of R₁₁ to each of R₁₃ may be the same or different.

According to one embodiment of the present disclosure, the formula 1 is represented by at least one of the following formulas 2 to 4.

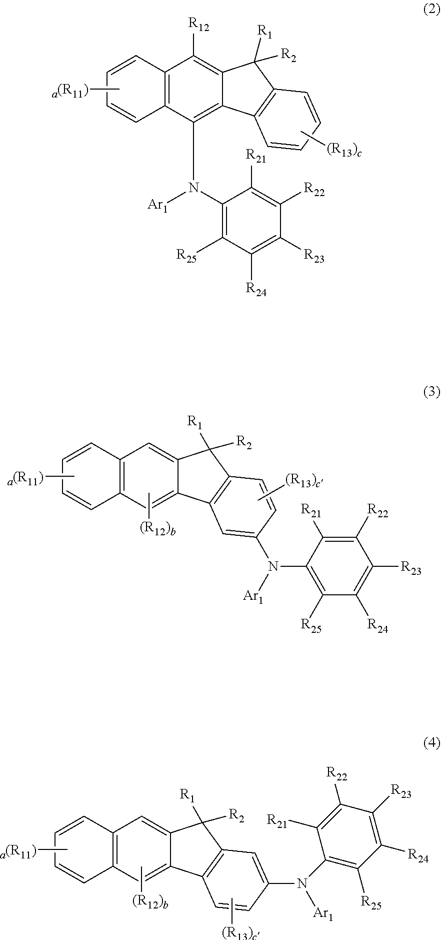

In formulas 2 to 4,
c′ represents an integer of 1 to 3, in which if c′ represents an integer of 2 or more, each of R₁₃ may be the same or different; and
R₁, R₂, R₁₁ to R₁₃, R₂₁ to R₂₅, Ar₁, a and b are as defined in formula 1.

According to one embodiment of the present disclosure, in the formula 1, the linking group a is represented by any one of the following structures.

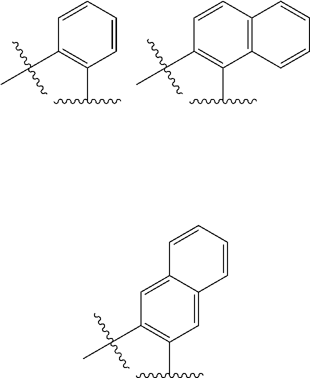 [SMILES: *c1ccccc1* ; *c1ccc2ccccc2c1* ; *c1cc2ccccc2cc1*]

In the structures, hydrogen may be substituted with at least one deuterium.

According to one embodiment of the present disclosure, in the formula 1, a substituted or unsubstituted (C12-C30)aryl comprising the linking group a in ortho form is represented by any one of the following structures.

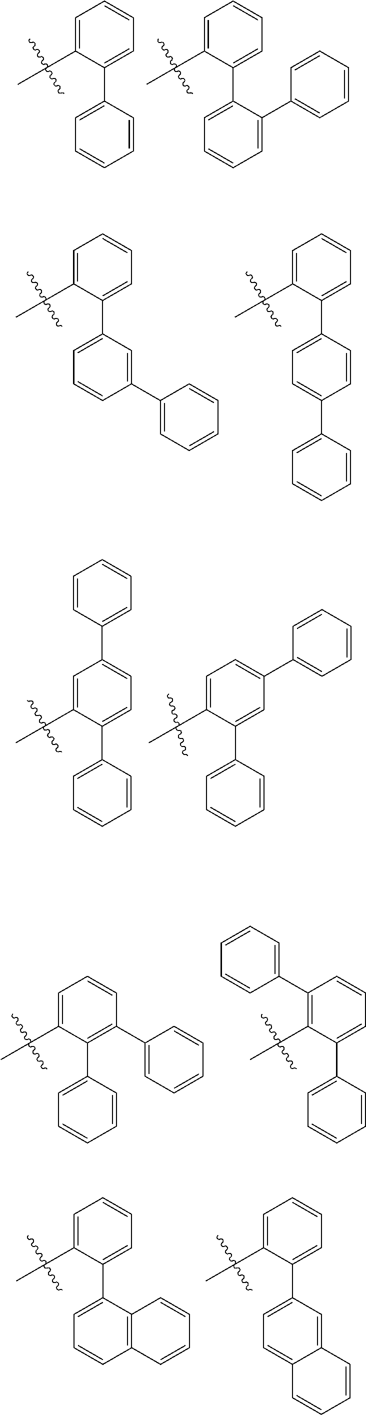

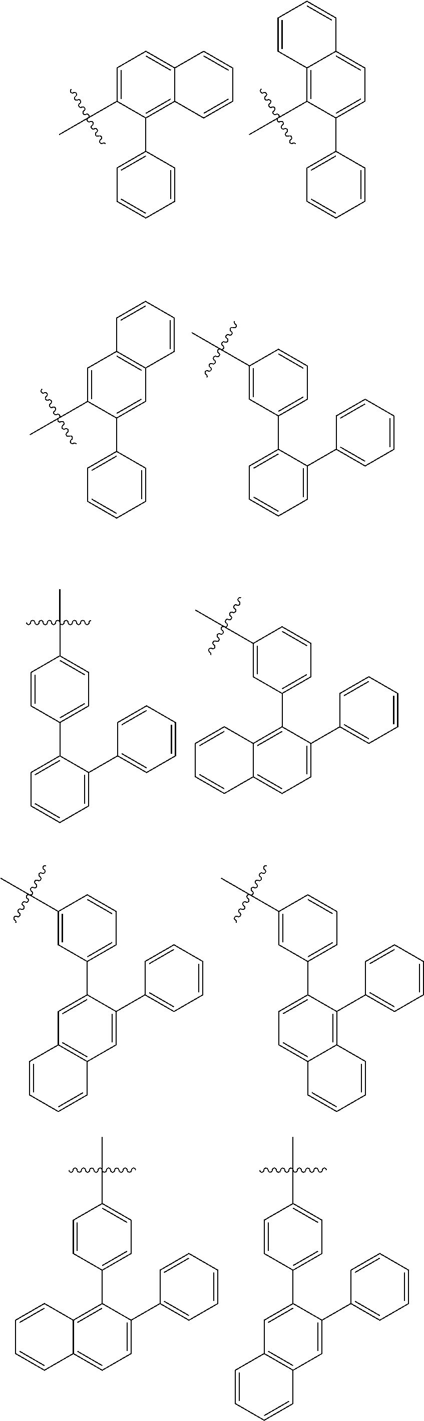

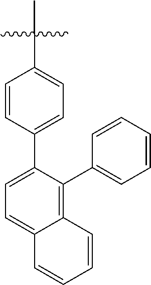 [SMILES: *c1ccc(-c2ccc3ccccc3c2-c2ccccc2)cc1]

In the structures, hydrogen may be substituted with at least one of deuterium and a methyl(s) unsubstituted or substituted with at least one deuterium.

The compound represented by formula 1 may be selected from the group consisting of the following compounds, but is not limited thereto.

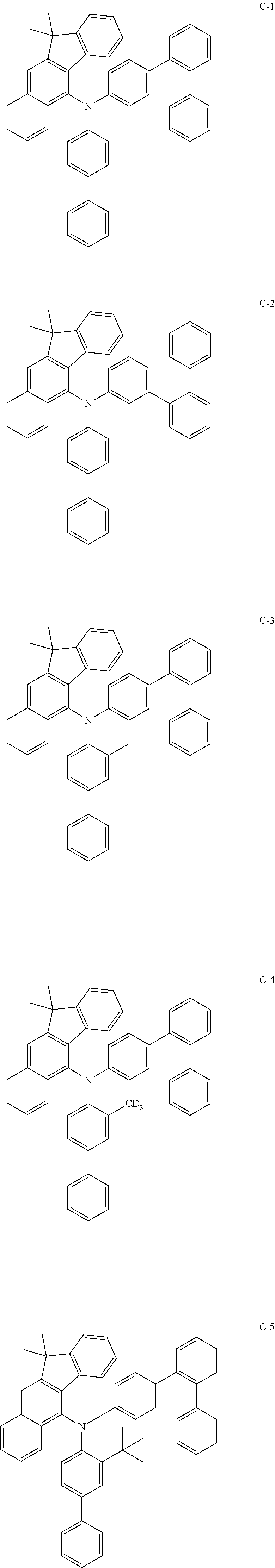 [SMILES: CC1(C)c2ccccc2-c2c1cc1ccccc1c2N(c1ccc(-c2ccccc2)cc1)c1ccc(-c2ccccc2-c2ccccc2)cc1 ; CC1(C)c2ccccc2-c2c1cc1ccccc1c2N(c1ccc(-c2ccccc2)cc1)c1cccc(-c2ccccc2-c2ccccc2)c1 ; Cc1cc(-c2ccccc2)ccc1N(c1ccc(-c2ccccc2-c2ccccc2)cc1)c1c2c(cc3ccccc13)C(C)(C)c1ccccc1-2 ; [2H]C([2H])([2H])c1cc(-c2ccccc2)ccc1N(c1ccc(-c2ccccc2-c2ccccc2)cc1)c1c2c(cc3ccccc13)C(C)(C)c1ccccc1-2 ; CC(C)(C)c1cc(-c2ccccc2)ccc1N(c1ccc(-c2ccccc2-c2ccccc2)cc1)c1c2c(cc3ccccc13)C(C)(C)c1ccccc1-2]

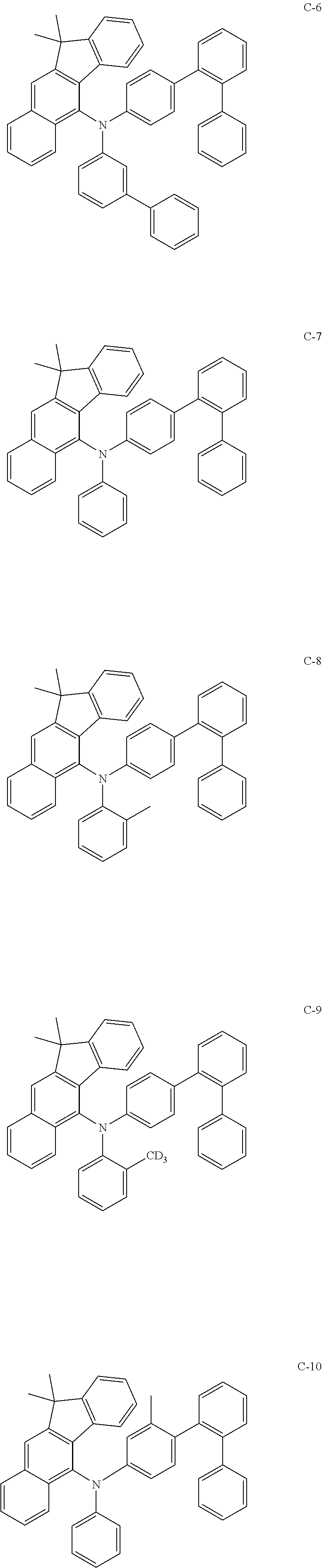 [SMILES: CC1(C)c2ccccc2-c2c1cc1ccccc1c2N(c1ccc(-c2ccccc2-c2ccccc2)cc1)c1cccc(-c2ccccc2)c1 ; CC1(C)c2ccccc2-c2c1cc1ccccc1c2N(c1ccccc1)c1ccc(-c2ccccc2-c2ccccc2)cc1 ; Cc1ccccc1N(c1ccc(-c2ccccc2-c2ccccc2)cc1)c1c2c(cc3ccccc13)C(C)(C)c1ccccc1-2 ; [2H]C([2H])([2H])c1ccccc1N(c1ccc(-c2ccccc2-c2ccccc2)cc1)c1c2c(cc3ccccc13)C(C)(C)c1ccccc1-2 ; Cc1cc(N(c2ccccc2)c2c3c(cc4ccccc24)C(C)(C)c2ccccc2-3)ccc1-c1ccccc1-c1ccccc1]

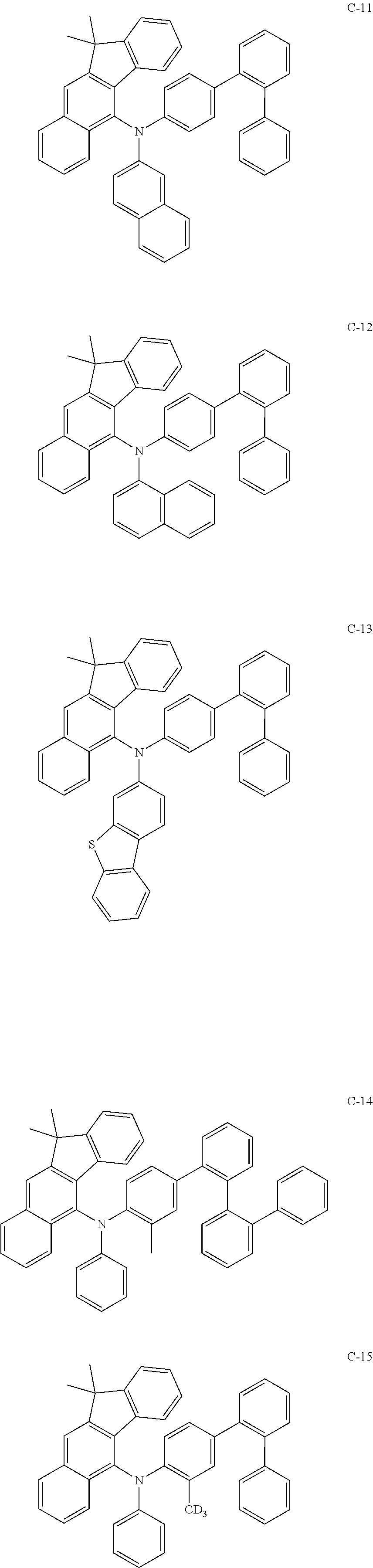 [SMILES: CC1(C)c2ccccc2-c2c1cc1ccccc1c2N(c1ccc(-c2ccccc2-c2ccccc2)cc1)c1ccc2ccccc2c1 ; CC1(C)c2ccccc2-c2c1cc1ccccc1c2N(c1ccc(-c2ccccc2-c2ccccc2)cc1)c1cccc2ccccc12 ; CC1(C)c2ccccc2-c2c1cc1ccccc1c2N(c1ccc(-c2ccccc2-c2ccccc2)cc1)c1ccc2c(c1)sc1ccccc12 ; Cc1cc(-c2ccccc2-c2ccccc2-c2ccccc2)ccc1N(c1ccccc1)c1c2c(cc3ccccc13)C(C)(C)c1ccccc1-2 ; [2H]C([2H])([2H])c1cc(-c2ccccc2-c2ccccc2)ccc1N(c1ccccc1)c1c2c(cc3ccccc13)C(C)(C)c1ccccc1-2]

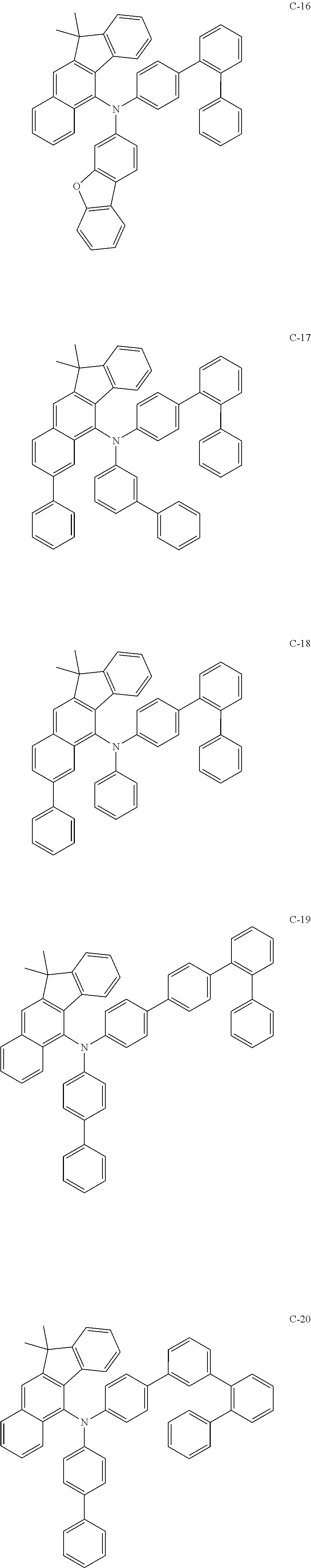

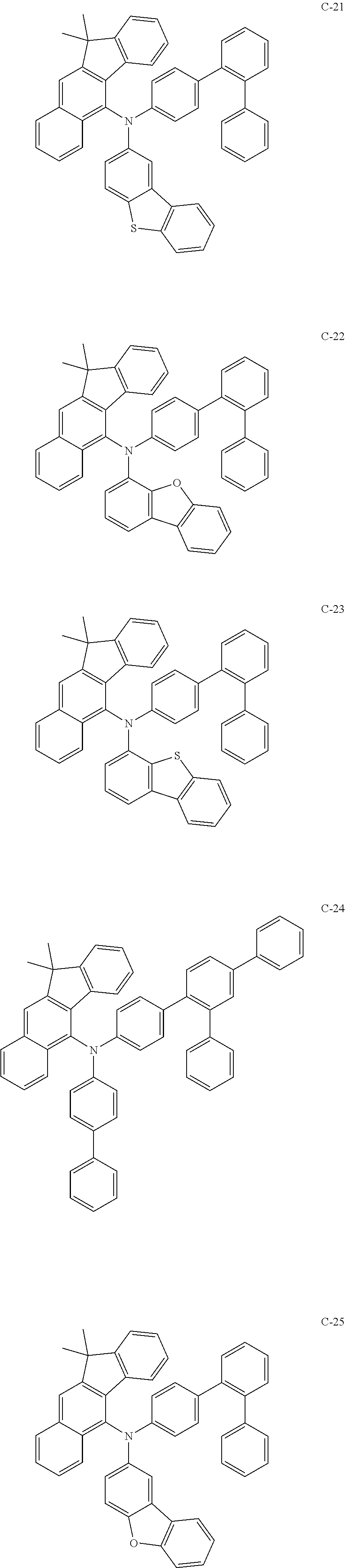 [SMILES: CC1(C)c2ccccc2-c2c1cc1ccccc1c2N(c1ccc(-c2ccccc2-c2ccccc2)cc1)c1ccc2sc3ccccc3c2c1 ; CC1(C)c2ccccc2-c2c1cc1ccccc1c2N(c1ccc(-c2ccccc2-c2ccccc2)cc1)c1cccc2c1oc1ccccc12 ; CC1(C)c2ccccc2-c2c1cc1ccccc1c2N(c1ccc(-c2ccccc2-c2ccccc2)cc1)c1cccc2c1sc1ccccc12 ; CC1(C)c2ccccc2-c2c1cc1ccccc1c2N(c1ccc(-c2ccccc2)cc1)c1ccc(-c2ccc(-c3ccccc3)cc2-c2ccccc2)cc1 ; CC1(C)c2ccccc2-c2c1cc1ccccc1c2N(c1ccc(-c2ccccc2-c2ccccc2)cc1)c1ccc2oc3ccccc3c2c1]

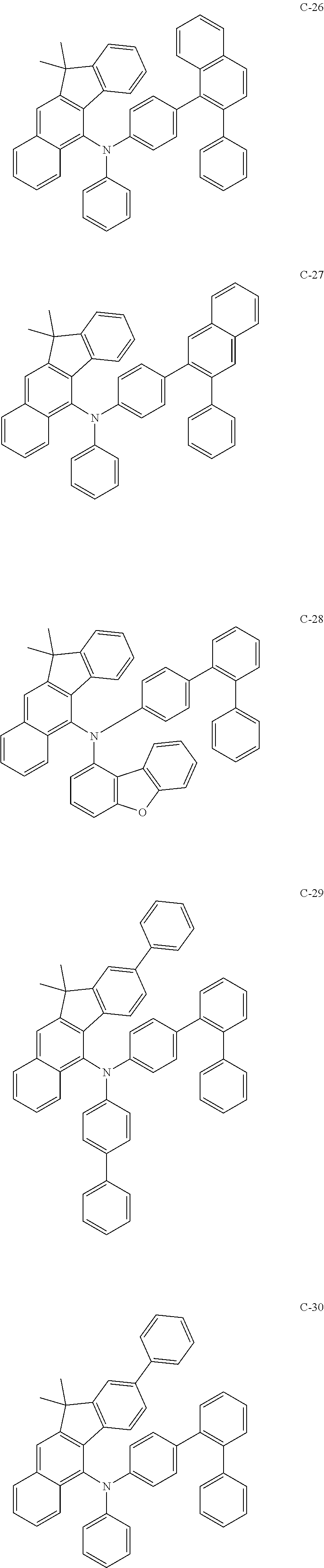 [SMILES: CC1(C)c2ccccc2-c2c1cc1ccccc1c2N(c1ccccc1)c1ccc(-c2c(-c3ccccc3)ccc3ccccc23)cc1 ; CC1(C)c2ccccc2-c2c1cc1ccccc1c2N(c1ccccc1)c1ccc(-c2cc3ccccc3cc2-c2ccccc2)cc1 ; CC1(C)c2ccccc2-c2c1cc1ccccc1c2N(c1ccc(-c2ccccc2-c2ccccc2)cc1)c1cccc2oc3ccccc3c12 ; CC1(C)c2cc(-c3ccccc3)ccc2-c2c1cc1ccccc1c2N(c1ccc(-c2ccccc2)cc1)c1ccc(-c2ccccc2-c2ccccc2)cc1 ; CC1(C)c2cc(-c3ccccc3)ccc2-c2c1cc1ccccc1c2N(c1ccccc1)c1ccc(-c2ccccc2-c2ccccc2)cc1]

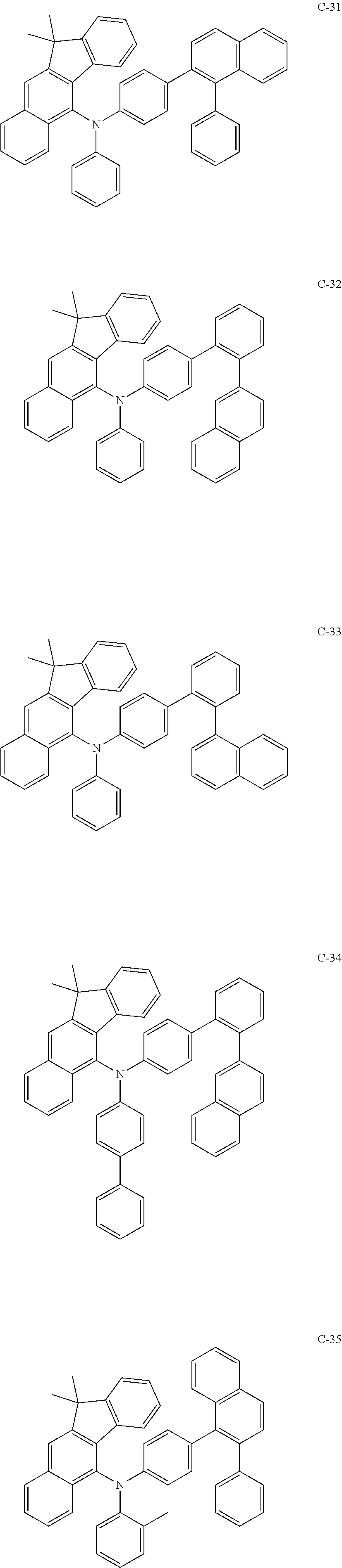 [SMILES: CC1(C)c2ccccc2-c2c1cc1ccccc1c2N(c1ccccc1)c1ccc(-c2ccc3ccccc3c2-c2ccccc2)cc1 ; CC1(C)c2ccccc2-c2c1cc1ccccc1c2N(c1ccccc1)c1ccc(-c2ccccc2-c2ccc3ccccc3c2)cc1 ; CC1(C)c2ccccc2-c2c1cc1ccccc1c2N(c1ccccc1)c1ccc(-c2ccccc2-c2cccc3ccccc23)cc1 ; CC1(C)c2ccccc2-c2c1cc1ccccc1c2N(c1ccc(-c2ccccc2)cc1)c1ccc(-c2ccccc2-c2ccc3ccccc3c2)cc1 ; Cc1ccccc1N(c1ccc(-c2c(-c3ccccc3)ccc3ccccc23)cc1)c1c2c(cc3ccccc13)C(C)(C)c1ccccc1-2]

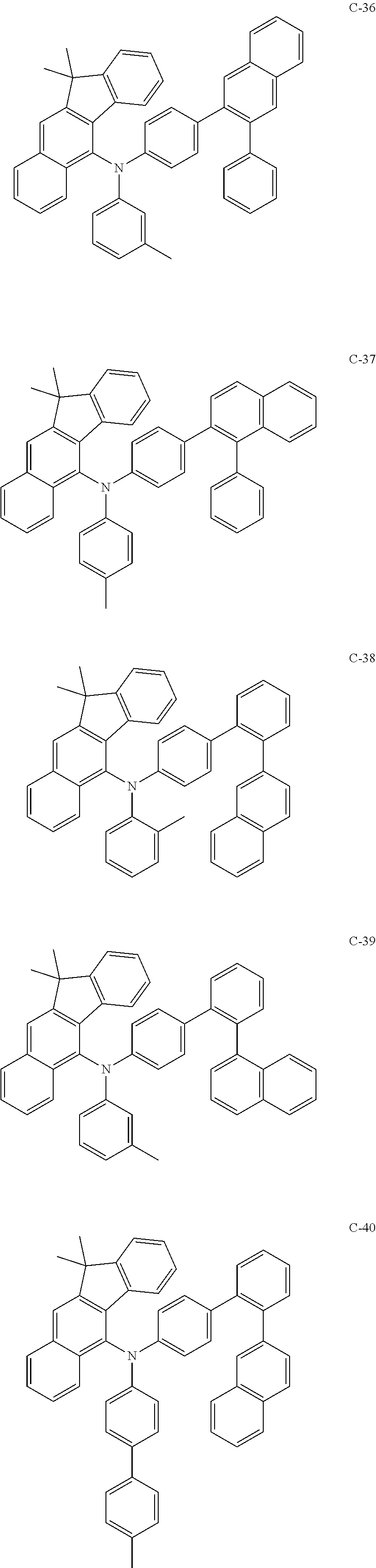 [SMILES: Cc1cccc(N(c2ccc(-c3cc4ccccc4cc3-c3ccccc3)cc2)c2c3c(cc4ccccc24)C(C)(C)c2ccccc2-3)c1 ; Cc1ccc(N(c2ccc(-c3ccc4ccccc4c3-c3ccccc3)cc2)c2c3c(cc4ccccc24)C(C)(C)c2ccccc2-3)cc1 ; Cc1ccccc1N(c1ccc(-c2ccccc2-c2ccc3ccccc3c2)cc1)c1c2c(cc3ccccc13)C(C)(C)c1ccccc1-2 ; Cc1cccc(N(c2ccc(-c3ccccc3-c3cccc4ccccc34)cc2)c2c3c(cc4ccccc24)C(C)(C)c2ccccc2-3)c1 ; Cc1ccc(-c2ccc(N(c3ccc(-c4ccccc4-c4ccc5ccccc5c4)cc3)c3c4c(cc5ccccc35)C(C)(C)c3ccccc3-4)cc2)cc1]

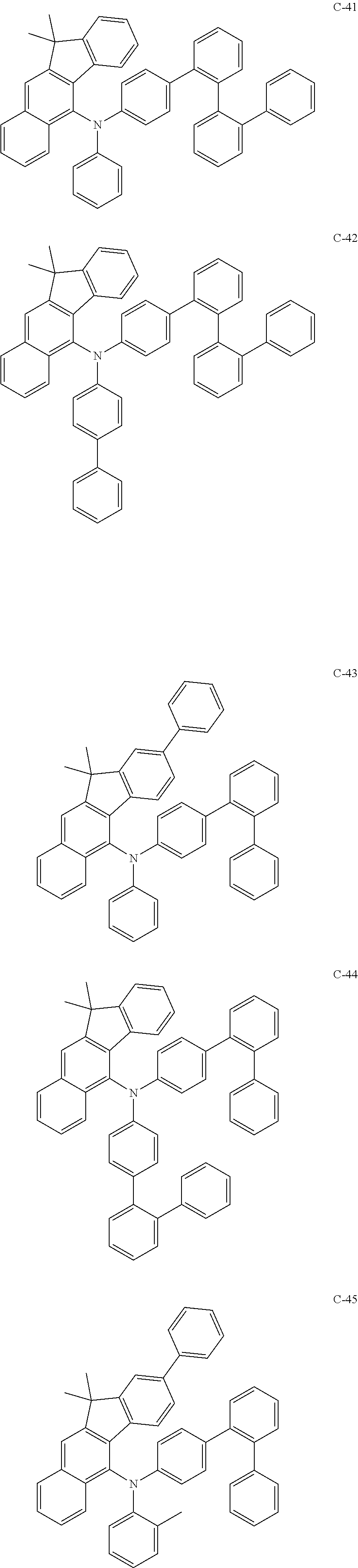 [SMILES: CC1(C)c2ccccc2-c2c1cc1ccccc1c2N(c1ccccc1)c1ccc(-c2ccccc2-c2ccccc2-c2ccccc2)cc1 ; CC1(C)c2ccccc2-c2c1cc1ccccc1c2N(c1ccc(-c2ccccc2)cc1)c1ccc(-c2ccccc2-c2ccccc2-c2ccccc2)cc1 ; CC1(C)c2cc(-c3ccccc3)ccc2-c2c1cc1ccccc1c2N(c1ccccc1)c1ccc(-c2ccccc2-c2ccccc2)cc1 ; CC1(C)c2ccccc2-c2c1cc1ccccc1c2N(c1ccc(-c2ccccc2-c2ccccc2)cc1)c1ccc(-c2ccccc2-c2ccccc2)cc1 ; Cc1ccccc1N(c1ccc(-c2ccccc2-c2ccccc2)cc1)c1c2c(cc3ccccc13)C(C)(C)c1cc(-c3ccccc3)ccc1-2]

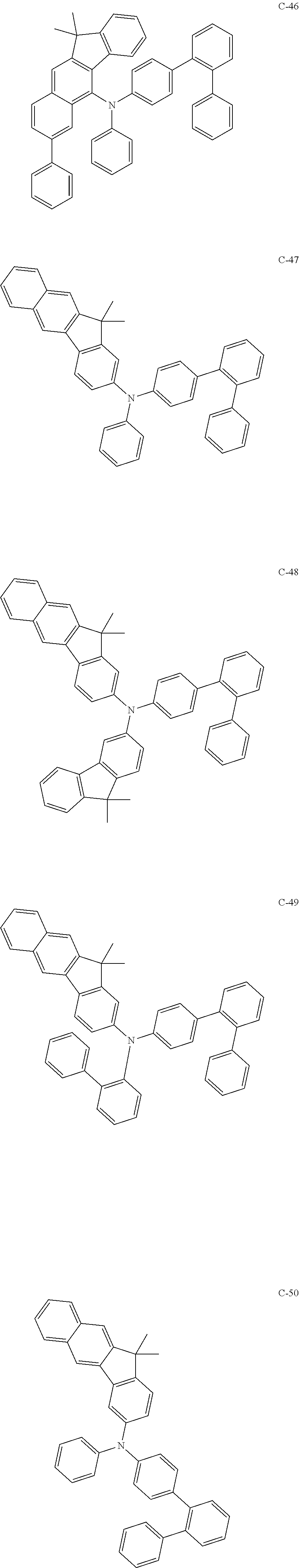 [SMILES: CC1(C)c2ccccc2-c2c1cc1ccc(-c3ccccc3)cc1c2N(c1ccccc1)c1ccc(-c2ccccc2-c2ccccc2)cc1 ; CC1(C)c2cc(N(c3ccccc3)c3ccc(-c4ccccc4-c4ccccc4)cc3)ccc2-c2cc3ccccc3cc21 ; CC1(C)c2ccccc2-c2cc(N(c3ccc(-c4ccccc4-c4ccccc4)cc3)c3ccc4c(c3)C(C)(C)c3cc5ccccc5cc3-4)ccc21 ; CC1(C)c2cc(N(c3ccc(-c4ccccc4-c4ccccc4)cc3)c3ccccc3-c3ccccc3)ccc2-c2cc3ccccc3cc21 ; CC1(C)c2ccc(N(c3ccccc3)c3ccc(-c4ccccc4-c4ccccc4)cc3)cc2-c2cc3ccccc3cc21]

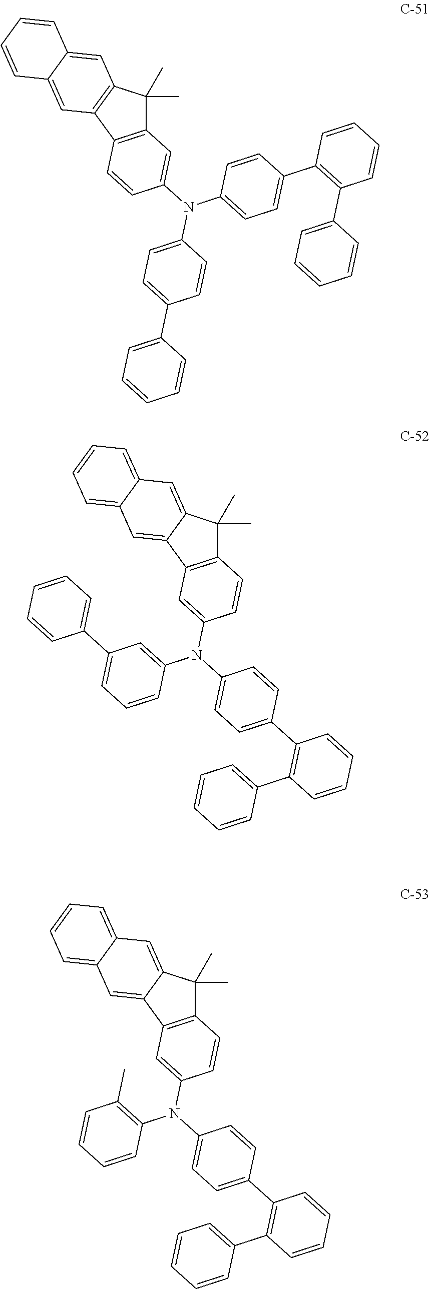 [SMILES: CC1(C)c2cc(N(c3ccc(-c4ccccc4)cc3)c3ccc(-c4ccccc4-c4ccccc4)cc3)ccc2-c2cc3ccccc3cc21 ; CC1(C)c2ccc(N(c3ccc(-c4ccccc4-c4ccccc4)cc3)c3cccc(-c4ccccc4)c3)cc2-c2cc3ccccc3cc21 ; Cc1ccccc1N(c1ccc(-c2ccccc2-c2ccccc2)cc1)c1ccc2c(c1)-c1cc3ccccc3cc1C2(C)C]

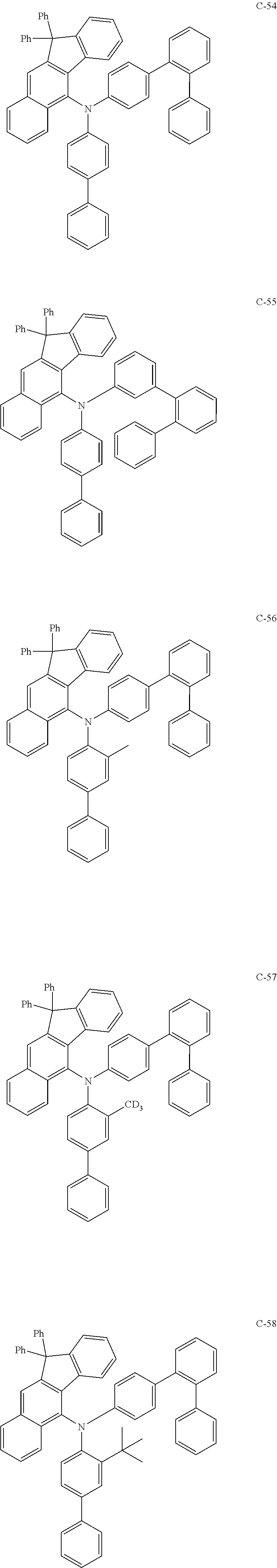

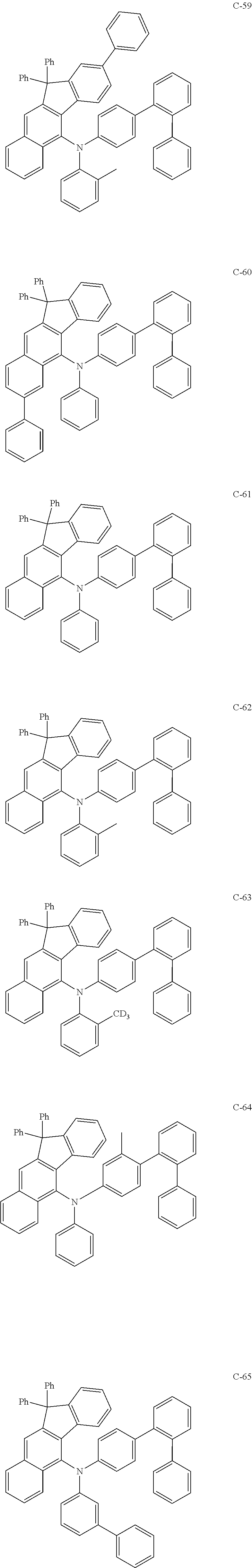

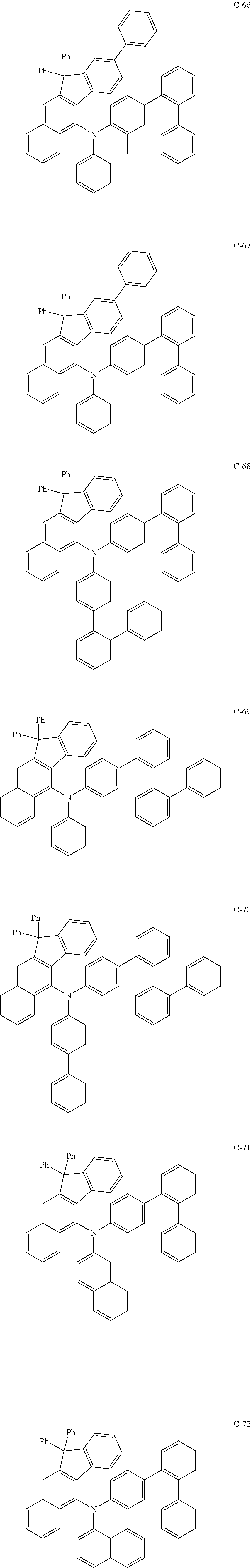

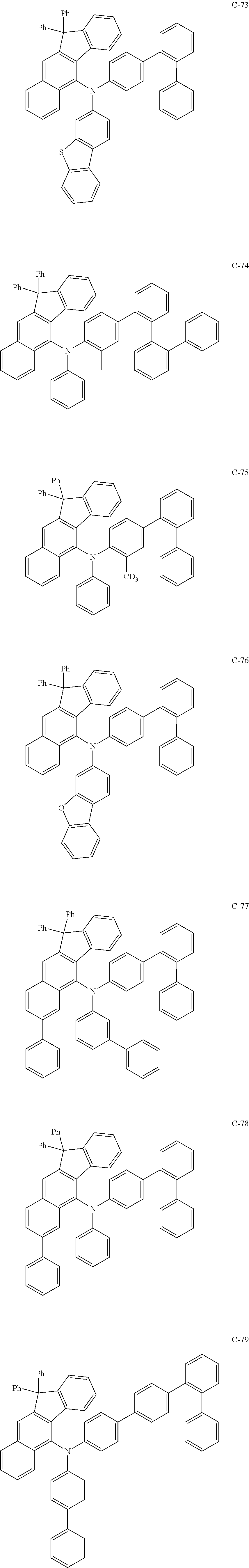

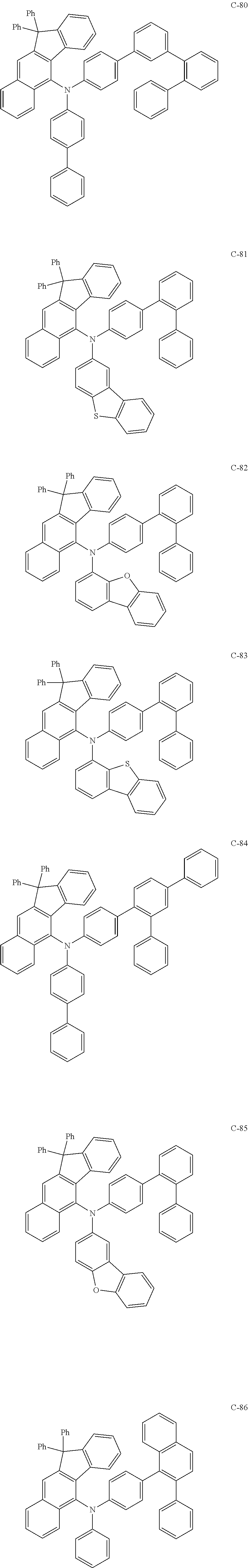

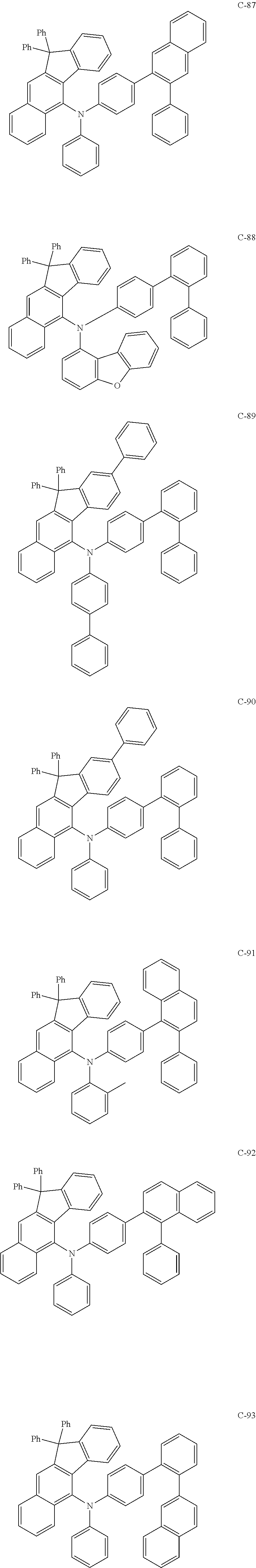

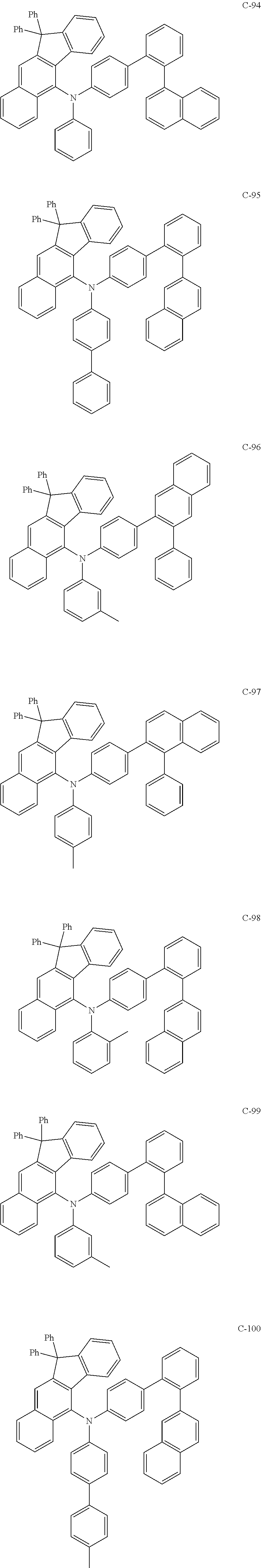

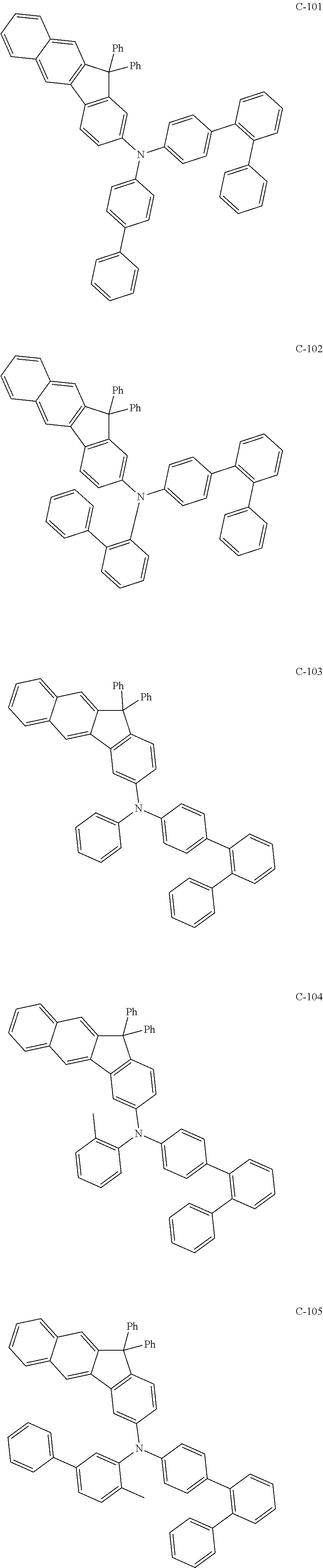 [SMILES: c1ccc(-c2ccc(N(c3ccc(-c4ccccc4-c4ccccc4)cc3)c3ccc4c(c3)C(c3ccccc3)(c3ccccc3)c3cc5ccccc5cc3-4)cc2)cc1 ; c1ccc(-c2ccccc2-c2ccc(N(c3ccc4c(c3)C(c3ccccc3)(c3ccccc3)c3cc5ccccc5cc3-4)c3ccccc3-c3ccccc3)cc2)cc1 ; c1ccc(-c2ccccc2-c2ccc(N(c3ccccc3)c3ccc4c(c3)-c3cc5ccccc5cc3C4(c3ccccc3)c3ccccc3)cc2)cc1 ; Cc1ccccc1N(c1ccc(-c2ccccc2-c2ccccc2)cc1)c1ccc2c(c1)-c1cc3ccccc3cc1C2(c1ccccc1)c1ccccc1 ; Cc1ccc(-c2ccccc2)cc1N(c1ccc(-c2ccccc2-c2ccccc2)cc1)c1ccc2c(c1)-c1cc3ccccc3cc1C2(c1ccccc1)c1ccccc1]

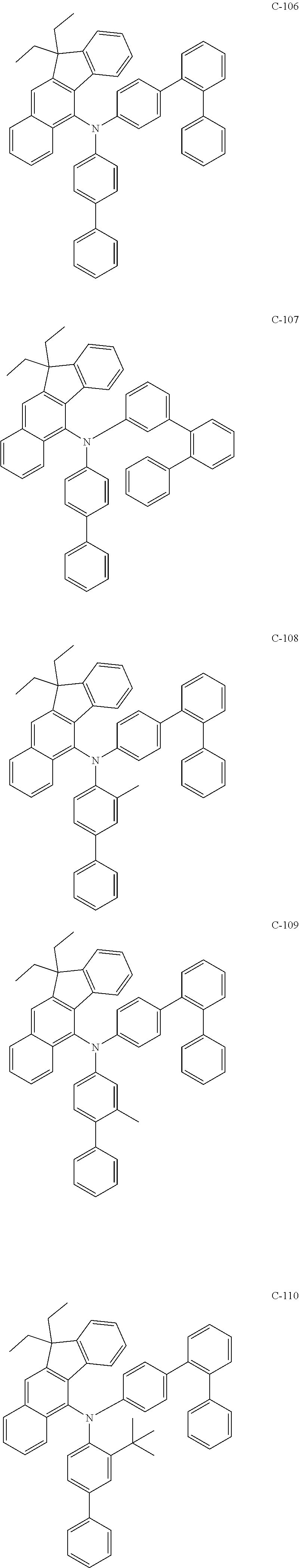

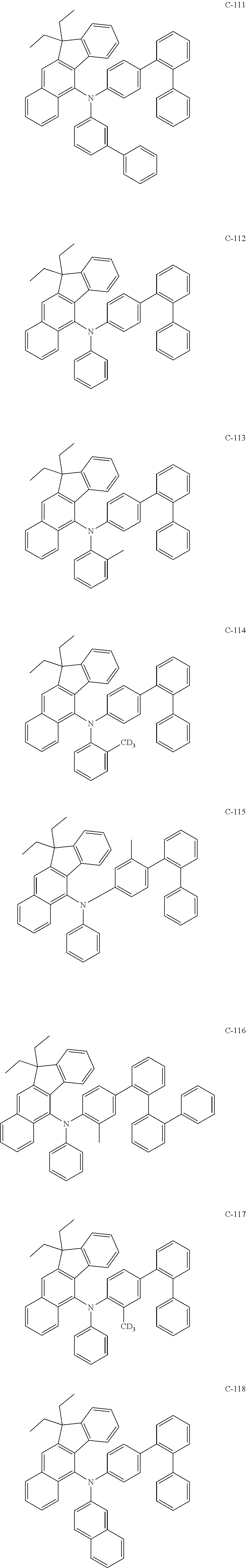

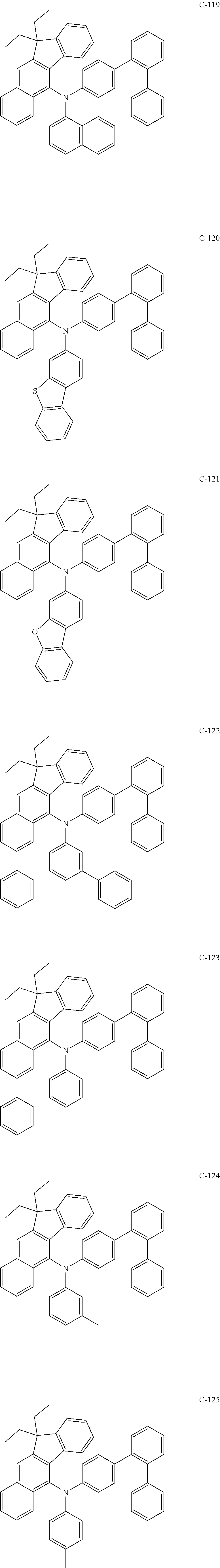

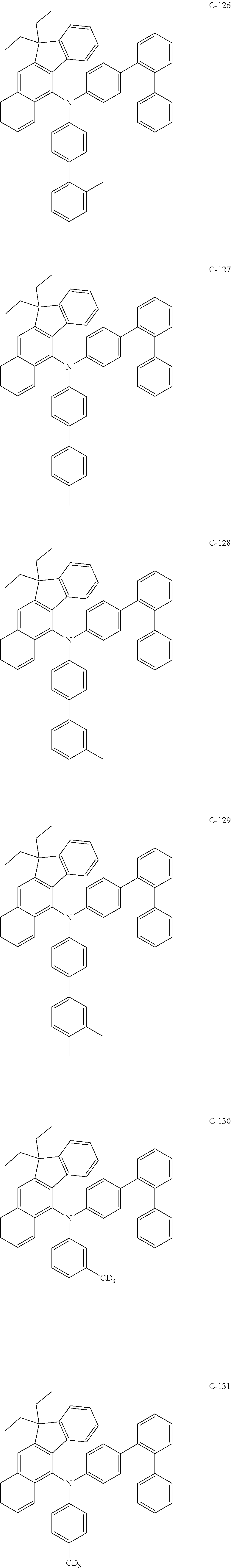

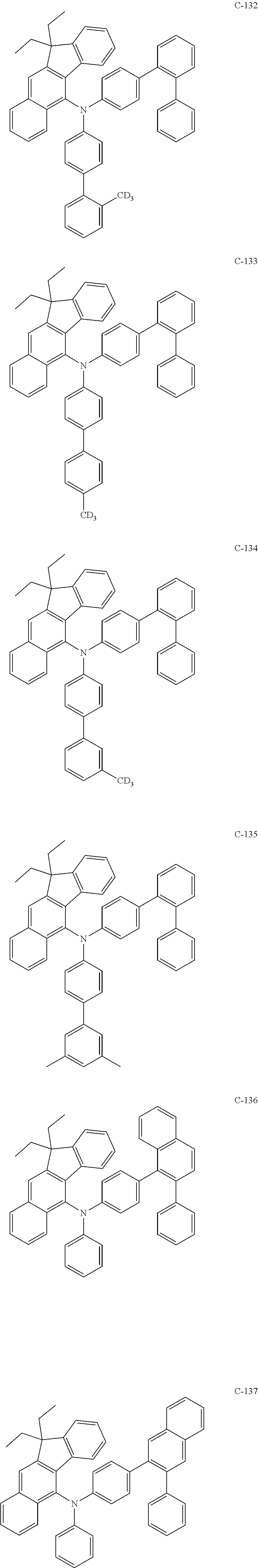

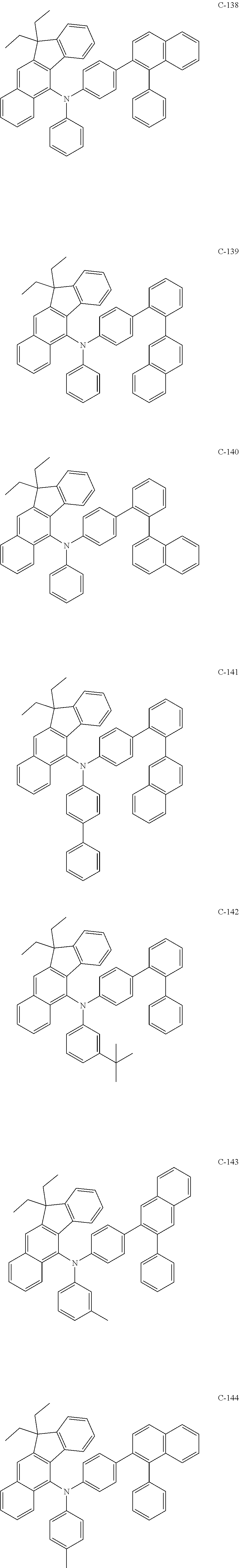

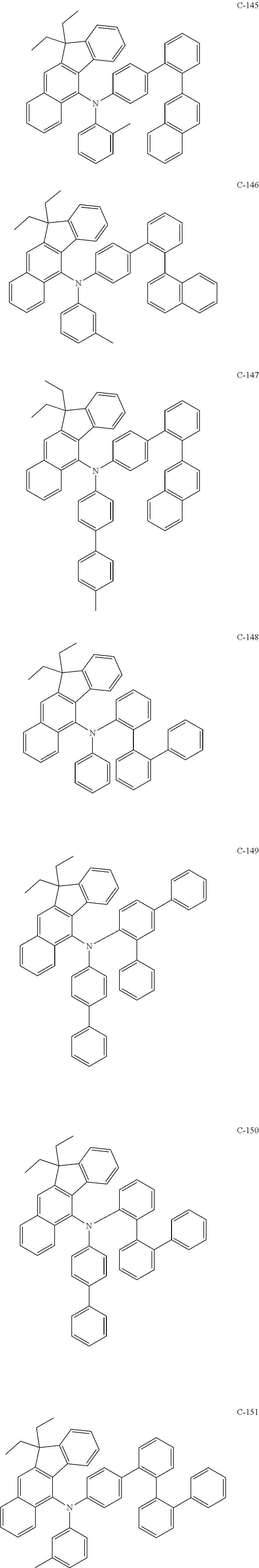

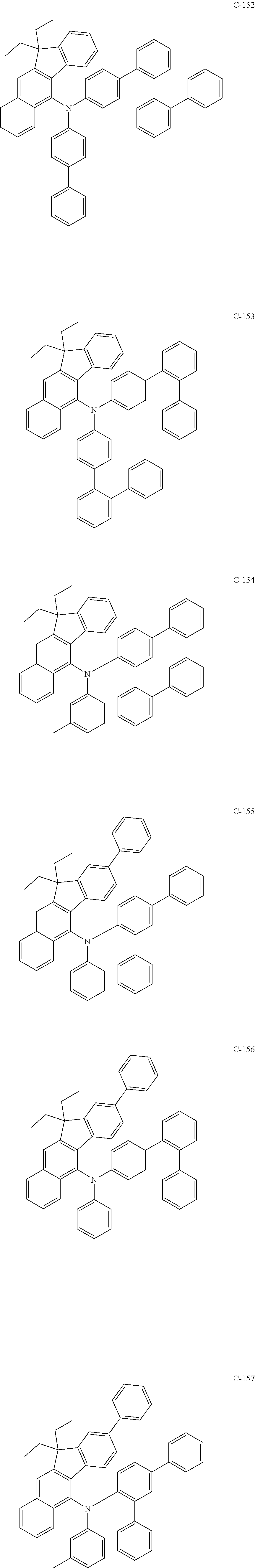

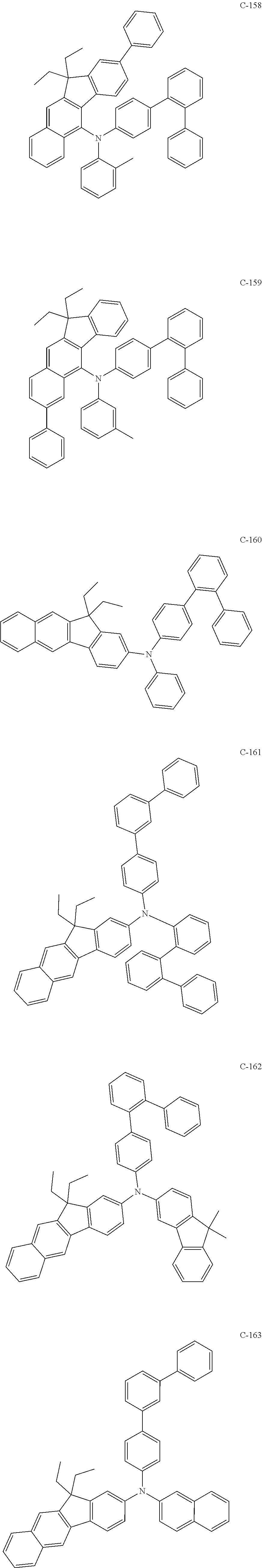

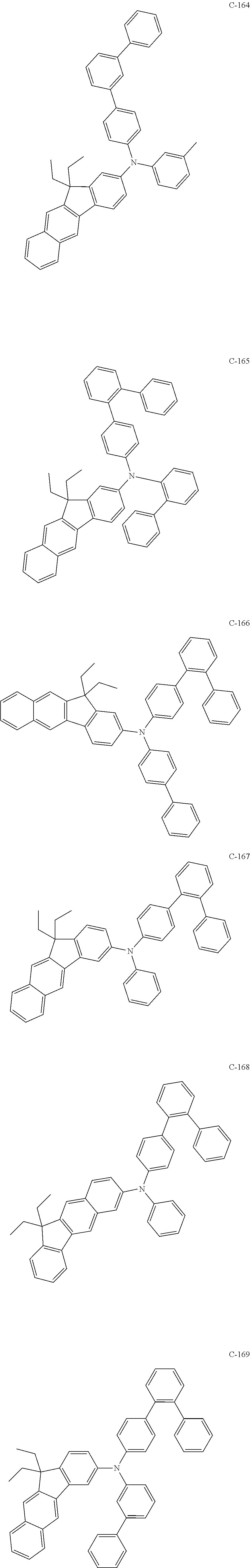

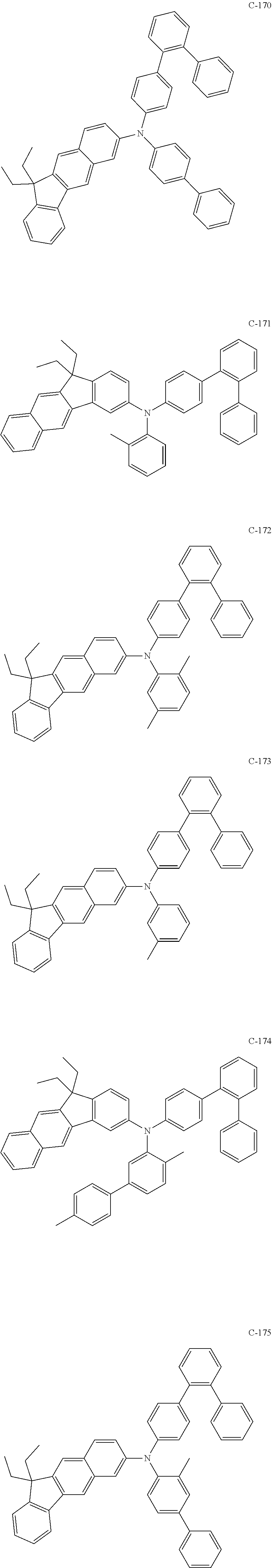 [SMILES: CCC1(CC)c2ccccc2-c2cc3cc(N(c4ccc(-c5ccccc5)cc4)c4ccc(-c5ccccc5-c5ccccc5)cc4)ccc3cc21 ; CCC1(CC)c2ccc(N(c3ccc(-c4ccccc4-c4ccccc4)cc3)c3ccccc3C)cc2-c2cc3ccccc3cc21 ; CCC1(CC)c2ccccc2-c2cc3cc(N(c4ccc(-c5ccccc5-c5ccccc5)cc4)c4cc(C)ccc4C)ccc3cc21 ; CCC1(CC)c2ccccc2-c2cc3cc(N(c4ccc(-c5ccccc5-c5ccccc5)cc4)c4cccc(C)c4)ccc3cc21 ; CCC1(CC)c2ccc(N(c3ccc(-c4ccccc4-c4ccccc4)cc3)c3cc(-c4ccc(C)cc4)ccc3C)cc2-c2cc3ccccc3cc21 ; CCC1(CC)c2ccccc2-c2cc3cc(N(c4ccc(-c5ccccc5-c5ccccc5)cc4)c4ccc(-c5ccccc5)cc4C)ccc3cc21]

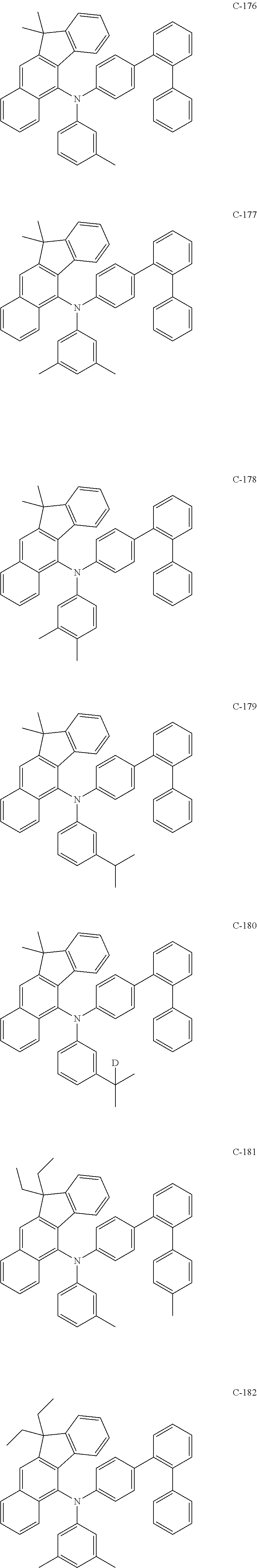

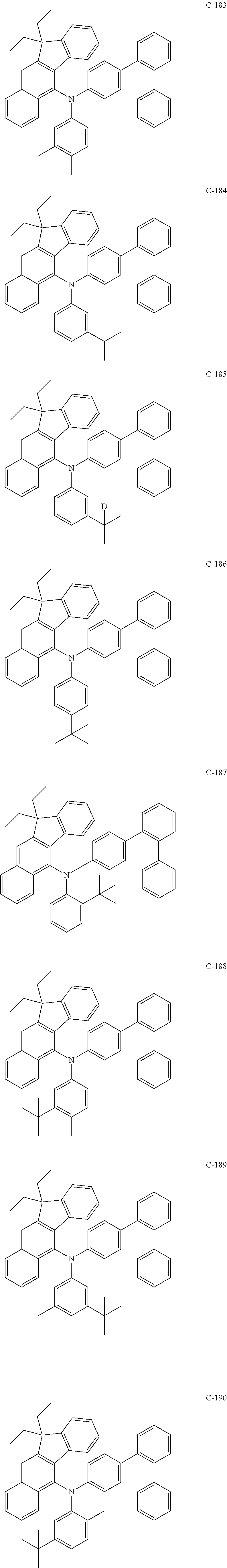

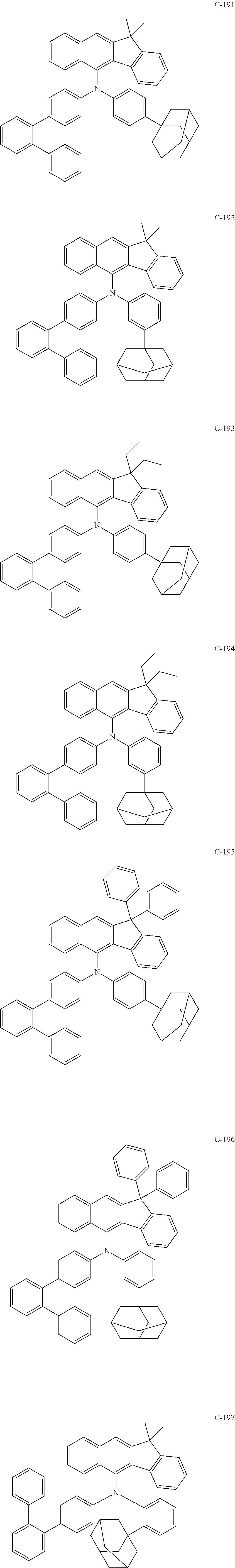

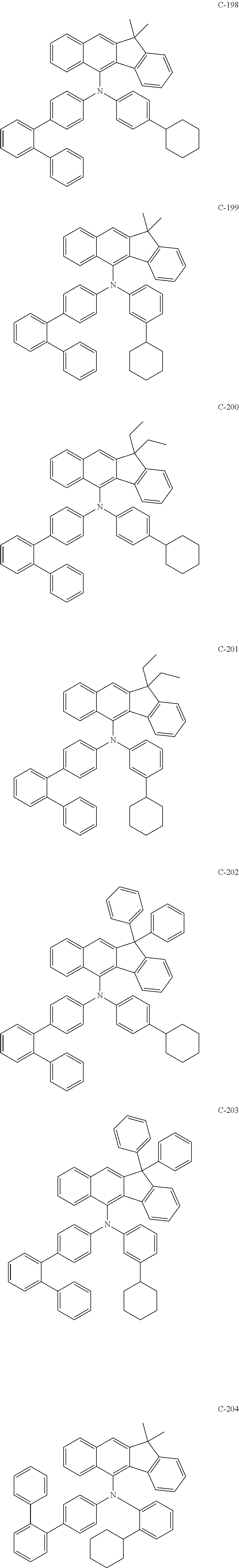

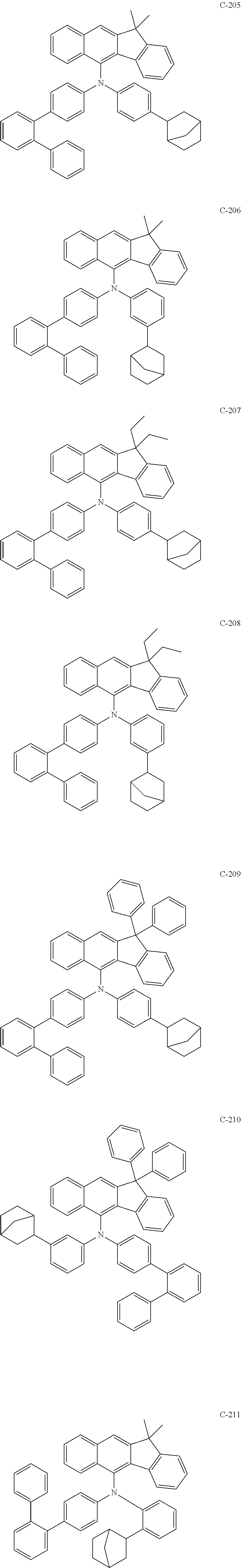

In the structures, Ph represents a phenyl group.

The compound represented by formula 1 according to the present disclosure may be produced by referring to the following reaction schemes, but is not limited thereto.

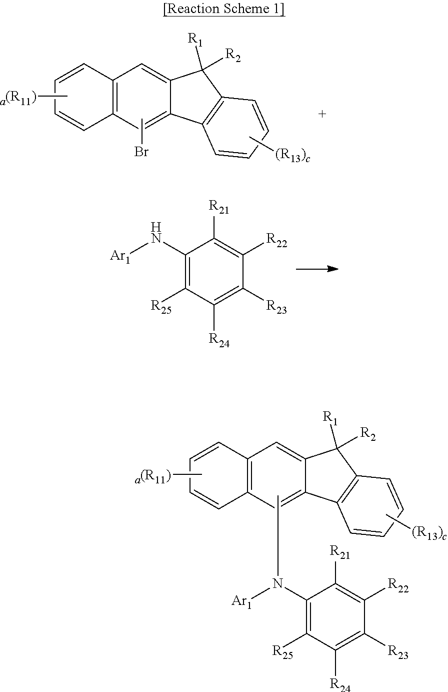

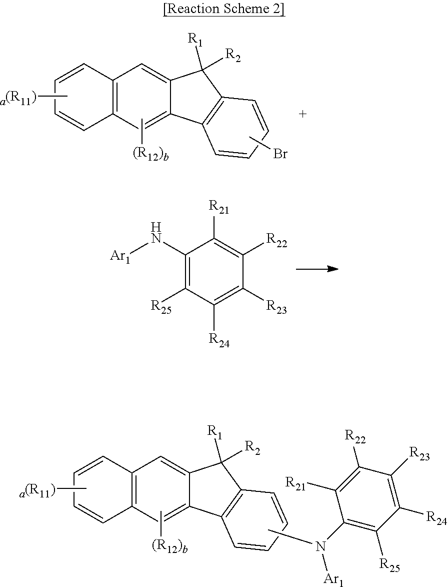

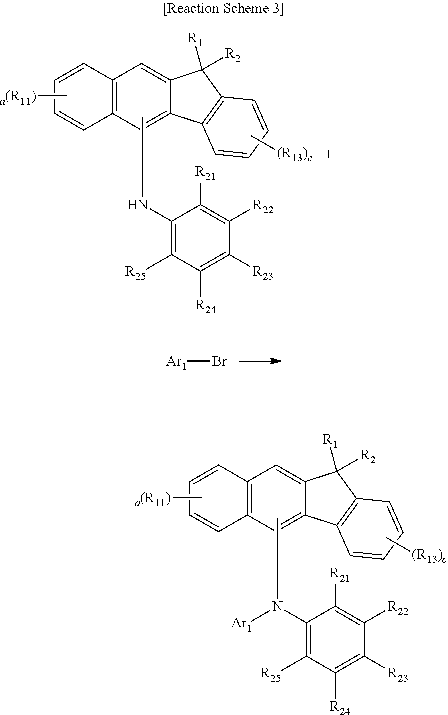

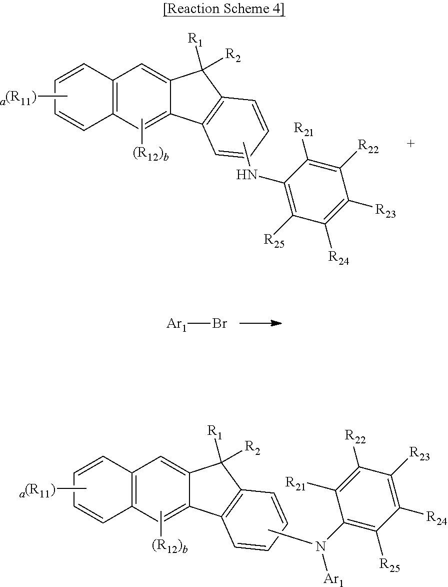

In reaction schemes 1 to 4, R₁, R₂, R₁₁ to R₁₃, R₂₁ to R₂₅, Ar₁, and a to c are as defined in formula 1.

The example of host compounds that can be used in combination with the organic electroluminescent compound of the present disclosure includes compounds represented by any one of the following formulas 11 to 13, but is not limited thereto.

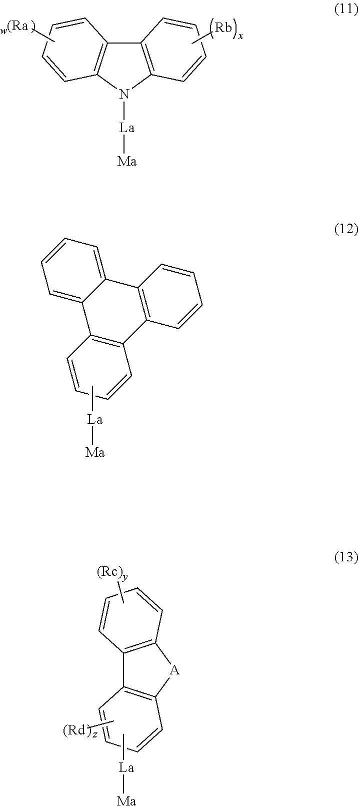

In formulas 11 to 13,
Ma represents a substituted or unsubstituted (C6-C30)aryl, a substituted or unsubstituted mono- or di-(C6-C30)arylamino, or a substituted or unsubstituted (3- to 30-membered)heteroaryl;
La represents a single bond, a substituted or unsubstituted (C6-C30)arylene, or a substituted or unsubstituted (3- to 30-membered)heteroarylene;
A represents S, O, N(Re) or C(Rf)(Rg);
Ra to Rd, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted (C1-C30)alkyl(C6-C30)arylamino, or a substituted or unsubstituted mono- or di-(C6-C30)arylamino; or may be linked to an adjacent substituent(s) to form a substituted or unsubstituted, mono- or polycyclic, (3- to 30-membered) alicyclic or aromatic ring, or a combination thereof, and the formed alicyclic or aromatic ring, or a combination thereof may contain at least one heteroatom selected from N, O, and S;
Re to Rg, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or Rf and Rg may be linked to each other to form a substituted or unsubstituted, mono- or polycyclic, (3- to 30-membered) alicyclic or aromatic ring, or a combination thereof, and the formed alicyclic or aromatic ring, or a combination thereof may contain at least one heteroatom selected from N, O, and S;
w to y, each independently, represent an integer of 1 to 4, and z represents an integer of 1 to 3; in which if w to z represent an integer of 2 or more, each of Ra to each of Rd may be the same or different; and
the heteroaryl(ene) comprises at least one heteroatoms selected from B, N, O, S, Si and P.

The compound represented by any one of formulas 11 to 13 according to the present disclosure may be produced by a synthetic method known to one skilled in the art, but is not limited thereto.

The present disclosure provides an organic electroluminescent material comprising the organic electroluminescent compound of formula 1 and an organic electroluminescent device comprising the organic electroluminescent material.

The present disclosure provides an organic electroluminescent material comprising the organic electroluminescent compound represented by formula 1 and the compound represented by any one of formulas 11 to 13 above, and an organic electroluminescent device comprising the organic electroluminescent material.

The organic electroluminescent material may be a hole transport material, a hole auxiliary material, an electron blocking material, a light-emitting material or a light-emitting auxiliary material, specifically a hole transport material, a hole auxiliary material, an electron blocking material, a light-emitting material or a light-emitting auxiliary material for a blue light-emitting organic electroluminescent device, in which if there are two or more hole transport layers, it may be a hole transport material (hole auxiliary material) included in a hole transport layer adjacent to a light-emitting layer.

The organic electroluminescent material may consist of the organic electroluminescent compound of the present disclosure alone, and may further comprise conventional materials included in an organic electroluminescent material.

The hole transport zone of the present disclosure may be composed of at least one layer from the group consisting of a hole transport layer, a hole injection layer, an electron blocking layer, and a hole auxiliary layer, and each of the layers may be further configured as a plurality of layers.

According to one embodiment of the present disclosure, the hole transport zone includes a hole transport layer. In addition, the hole transport zone may comprise a hole transport layer, and may further comprise at least one layer among a hole injection layer, an electron blocking layer and a hole auxiliary layer.

The organic electroluminescent device of the present disclosure comprises a first electrode; a second electrode; and at least one organic layer between the first electrode and the second electrode, wherein the organic layer may comprise at least one organic electroluminescent compound of formula 1. One of the first electrode and the second electrode may be an anode, and the other may be a cathode. In addition, the organic layer may comprise a light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

The organic electroluminescent compound of formula 1 according to the present disclosure may be comprised in at least one of a light-emitting layer, a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. It may preferably be comprised in at least one of a hole transport layer, a hole auxiliary layer, an electron blocking layer, a light-emitting auxiliary layer, and a light-emitting layer. If there are two or more hole transport layers, it can be used in at least one of them. For example, when used in a hole transport layer, the organic electroluminescent compound of the present disclosure may be comprised as a hole transport material. In addition, when used in a light-emitting layer, the organic electroluminescent compound of the present disclosure may be comprised as a host material.

The light-emitting layer may include at least one host and at least one dopant. If necessary, the light-emitting layer may include a co-host material, that is, two or more host materials. The organic electroluminescent compound of the present disclosure may be used as a co-host material.

The host used herein may be a phosphorescent host compound or a fluorescent host compound, and these host compounds are not particularly limited.

The dopant comprised in the organic electroluminescent device of the present disclosure may be at least one phosphorescent or fluorescent dopant, and is preferably a phosphorescent dopant. The phosphorescent dopant materials applied to the organic electroluminescent device according to the present disclosure are not particularly limited, but may be selected from metallated complex compounds of iridium (Ir), osmium (Os), copper (Cu), and platinum (Pt), preferably selected from ortho-metallated complex compounds of iridium (Ir), osmium (Os), copper (Cu), and platinum (Pt), and more preferably an ortho-metallated iridium complex compound.

The dopant comprised in the organic electroluminescent device of the present disclosure may be a compound represented by the following formula 101, but is not limited thereto.

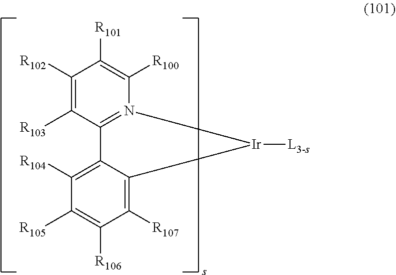

In formula 101,
L is any one selected from the following structures 1 to 3;

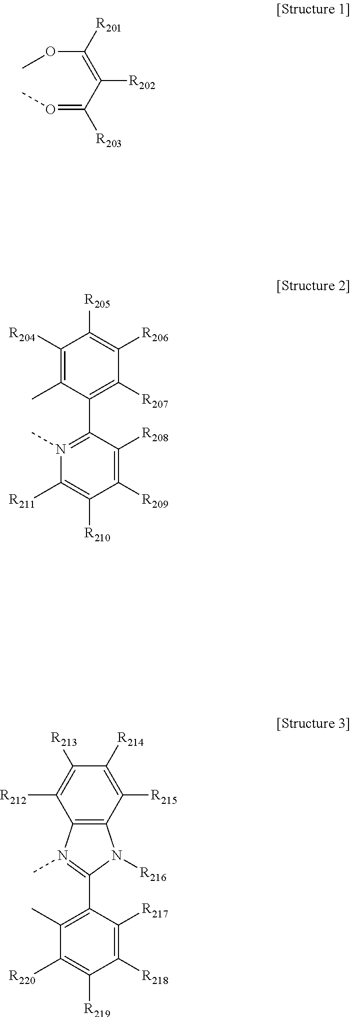

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, isoquinoline, benzofuropyridine, benzothienopyridine, indenopyridine, benzofuroquinoline, benzothienoquinoline, or indenoquinoline, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and
s represents an integer of 1 to 3.

The specific examples of the dopant compound are as follows, but are not limited thereto.

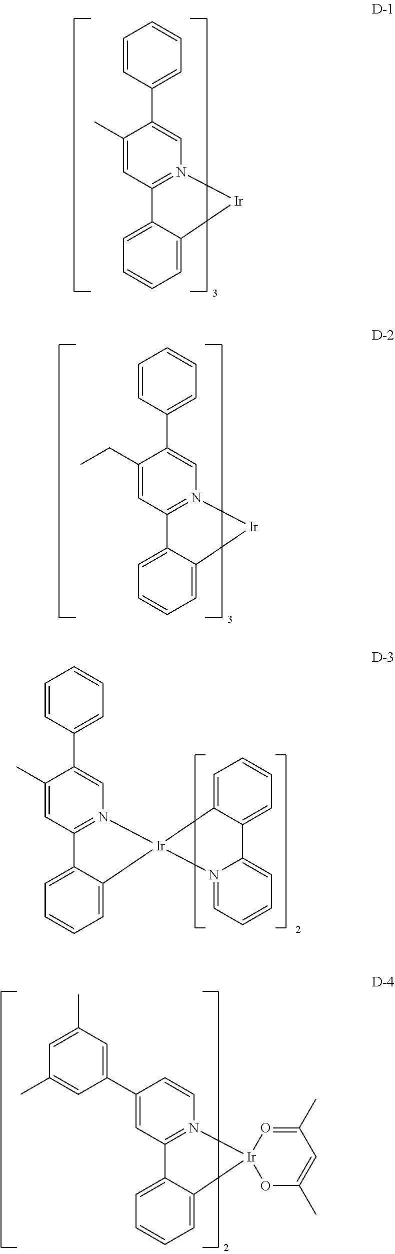

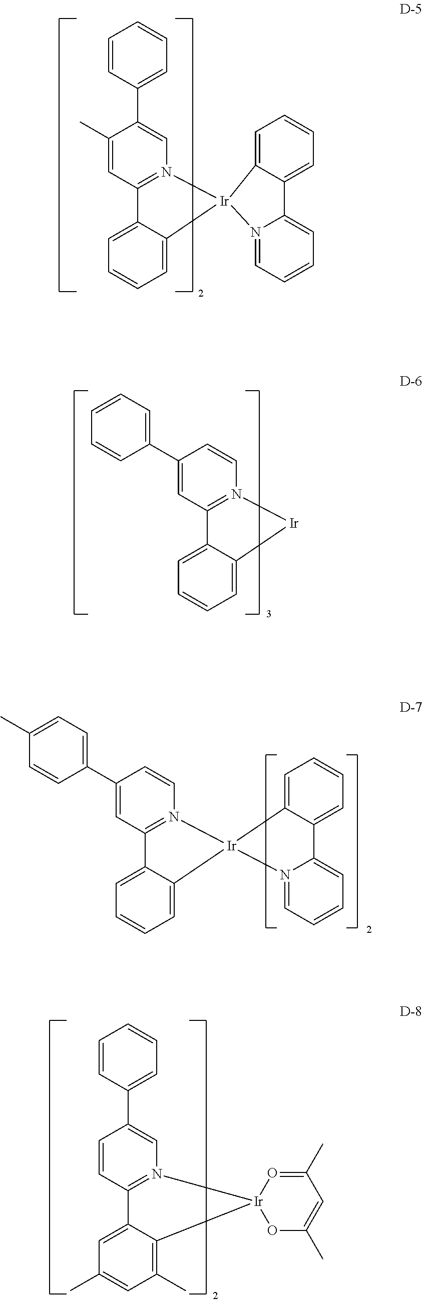

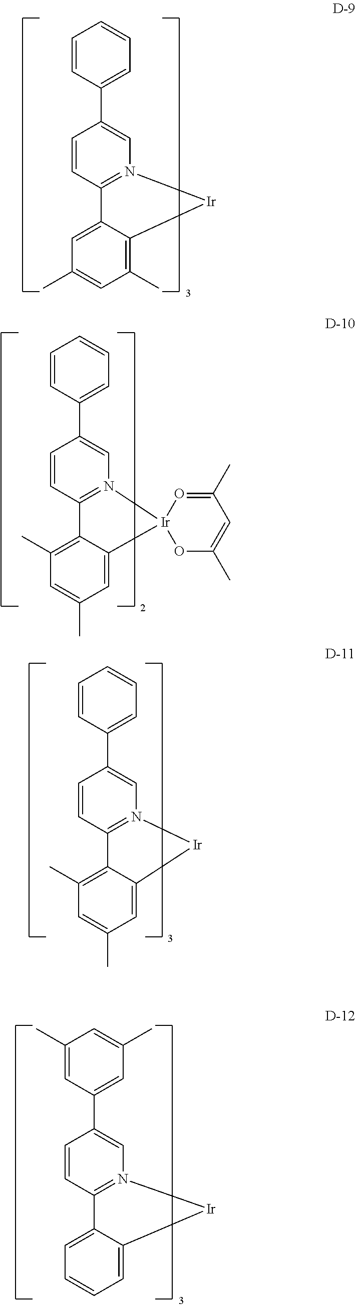

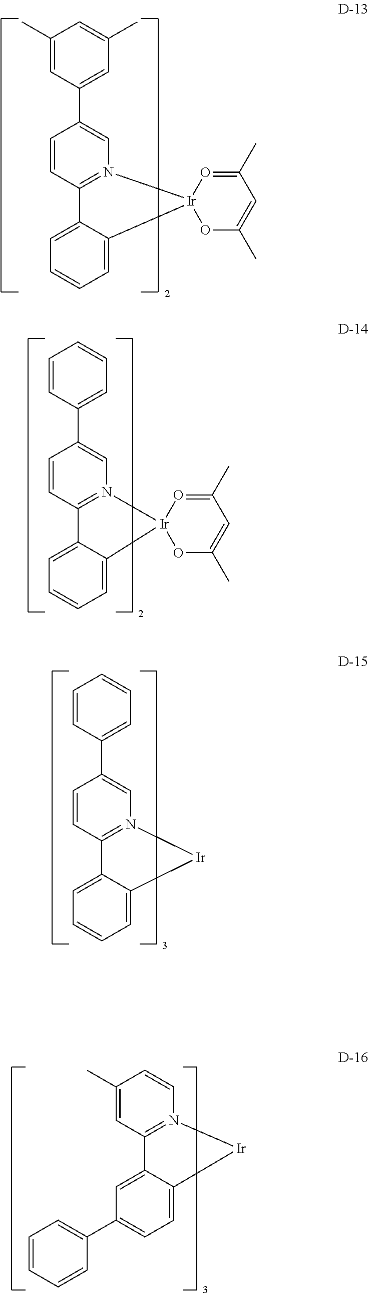

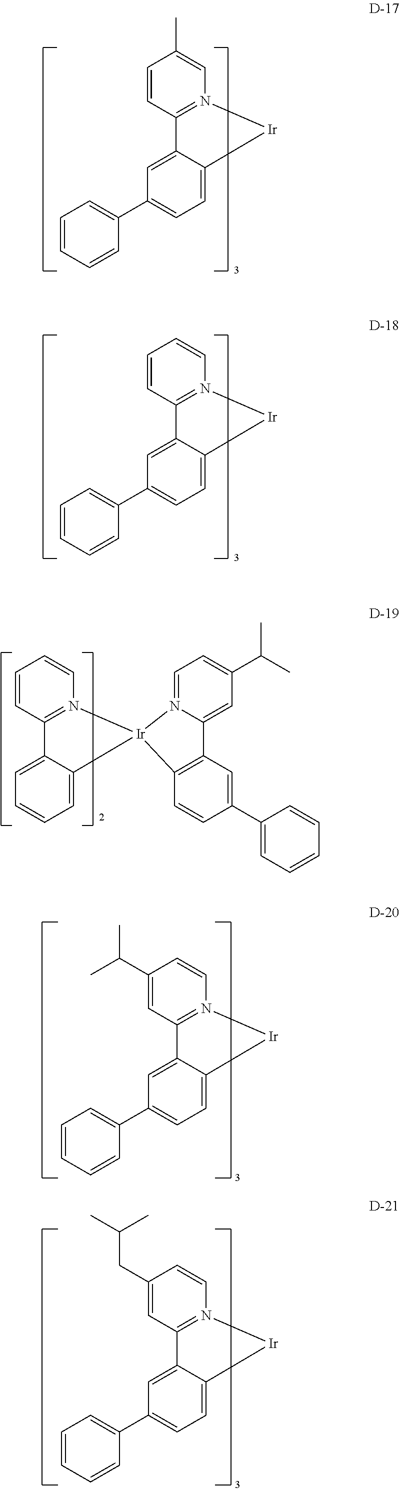

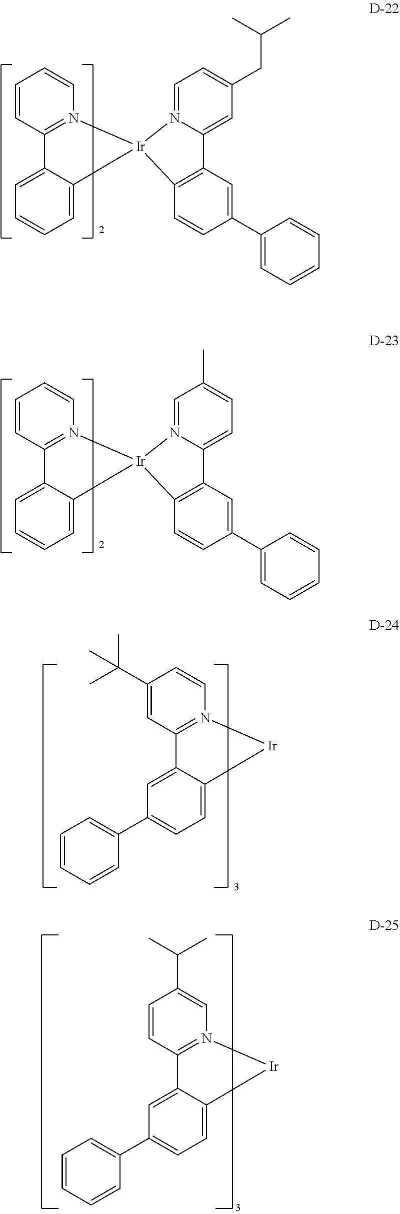

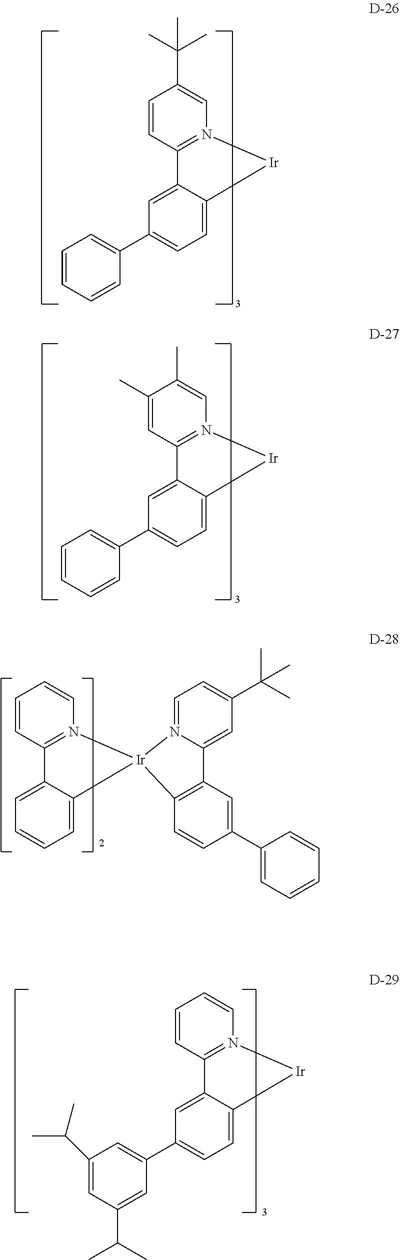

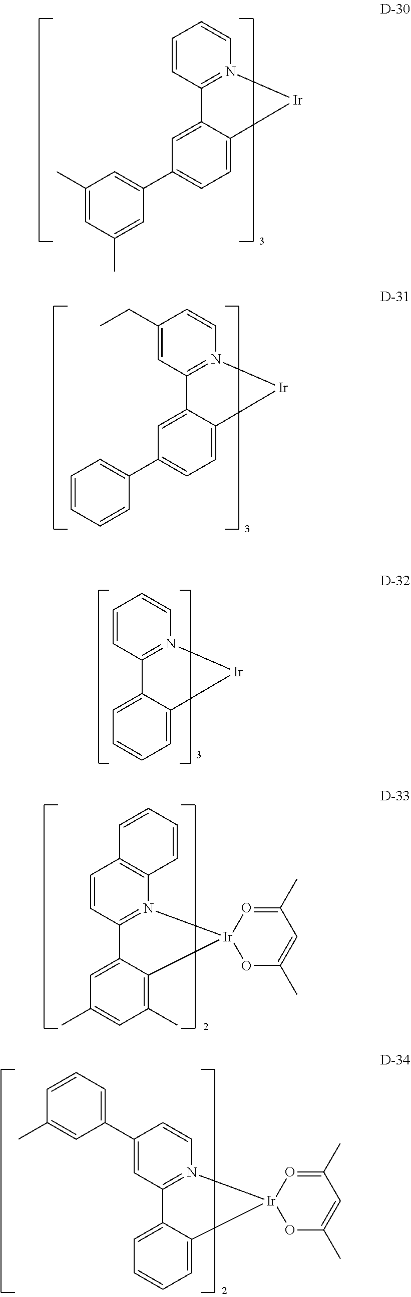

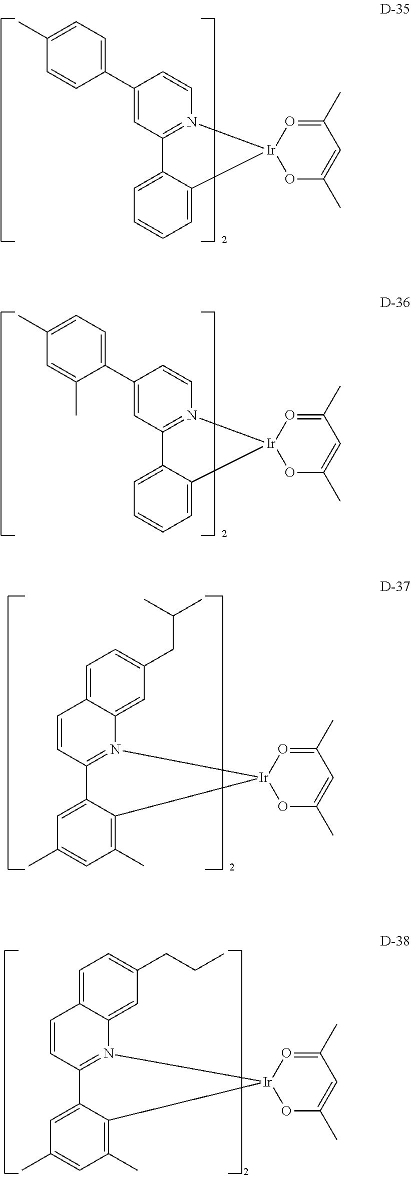

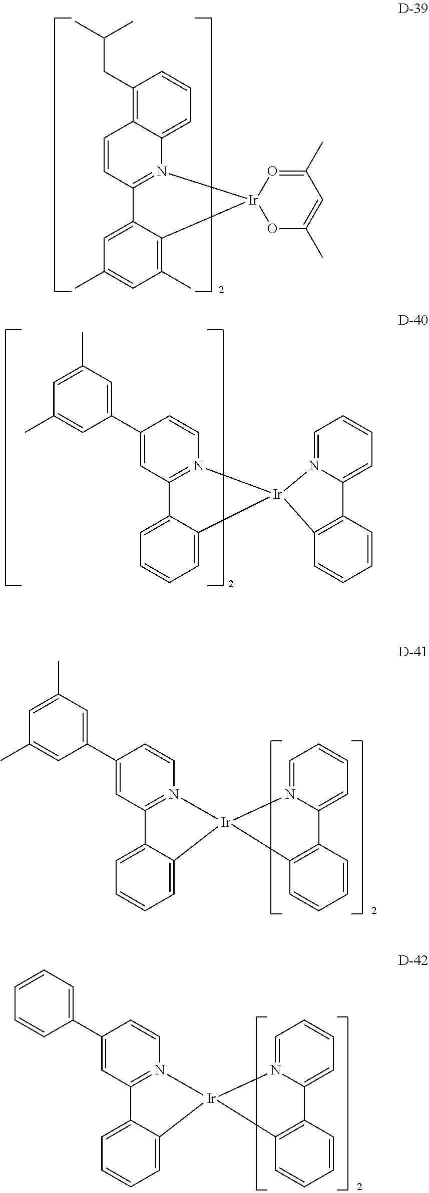

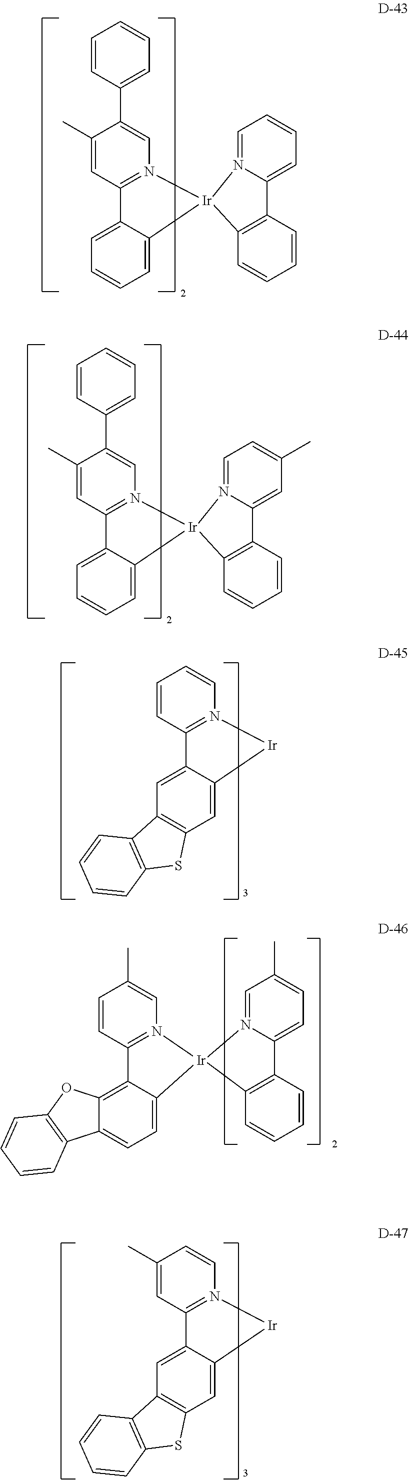

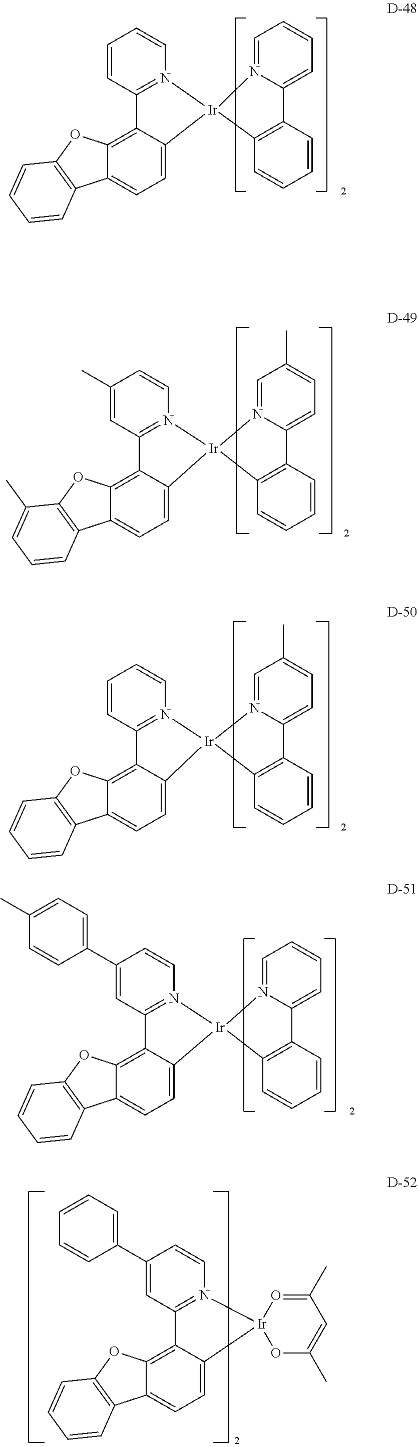

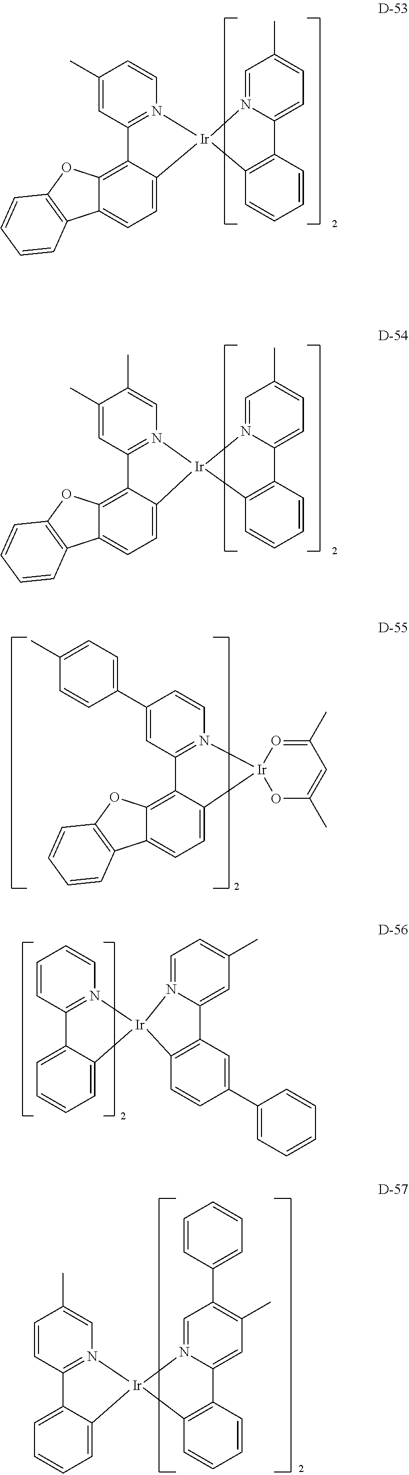

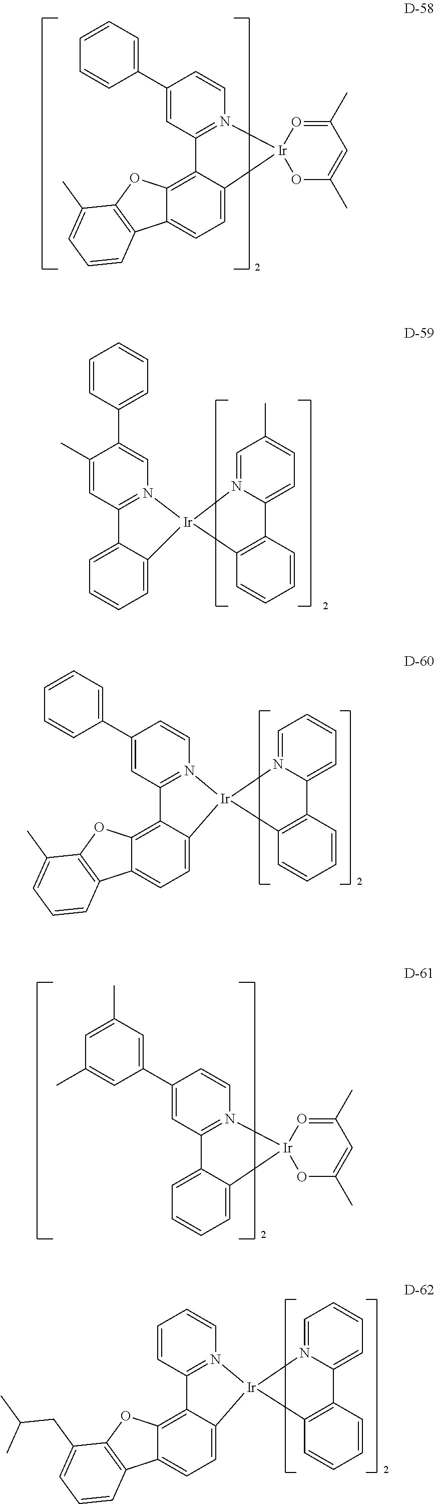

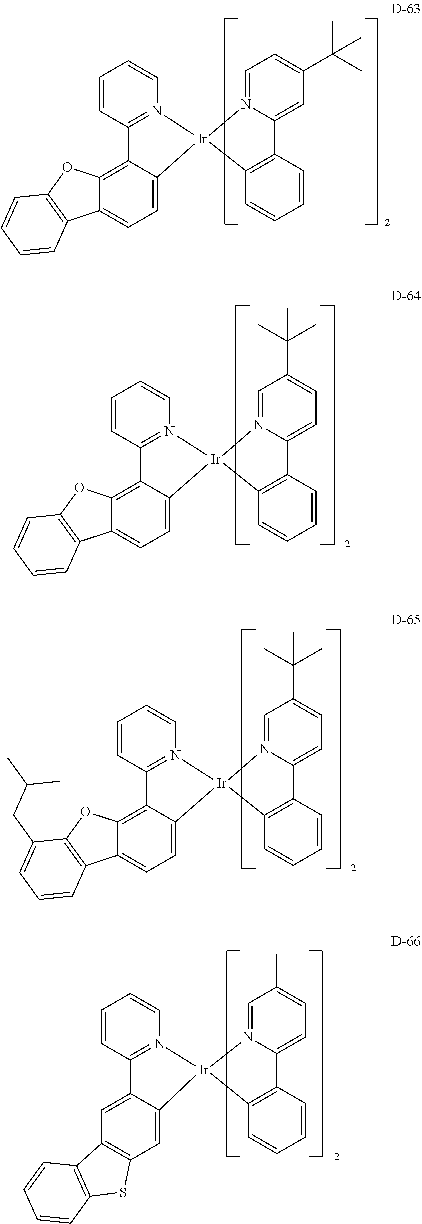

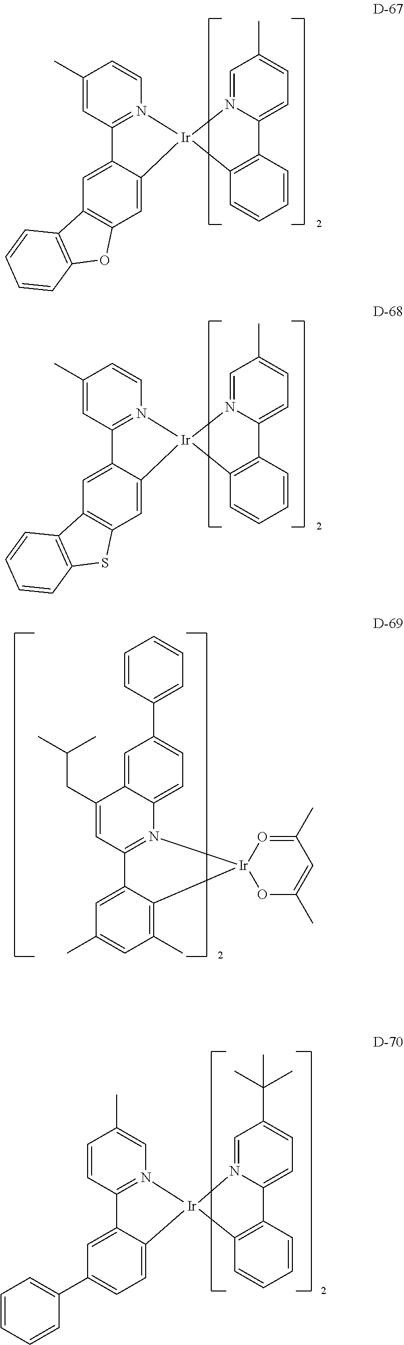

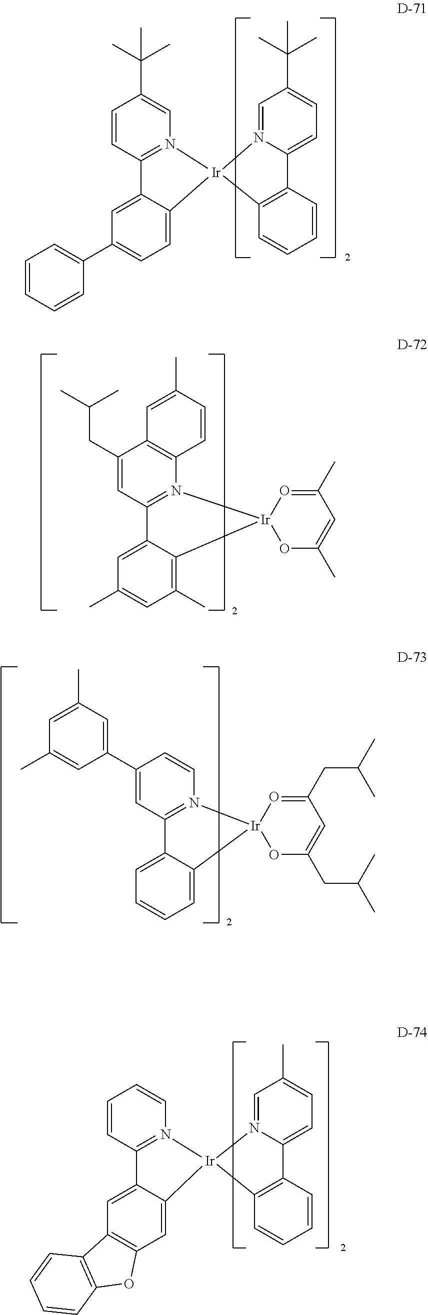

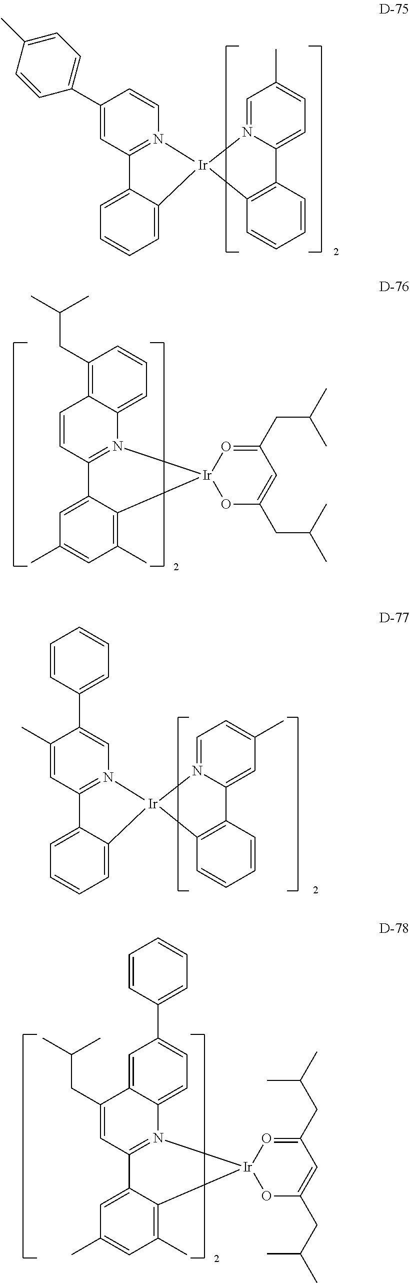

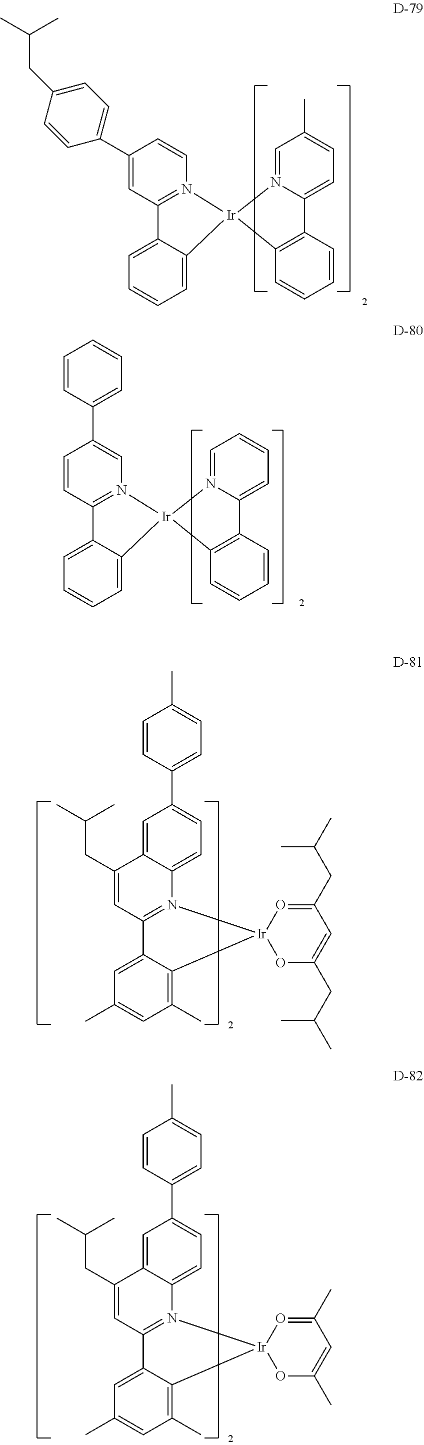

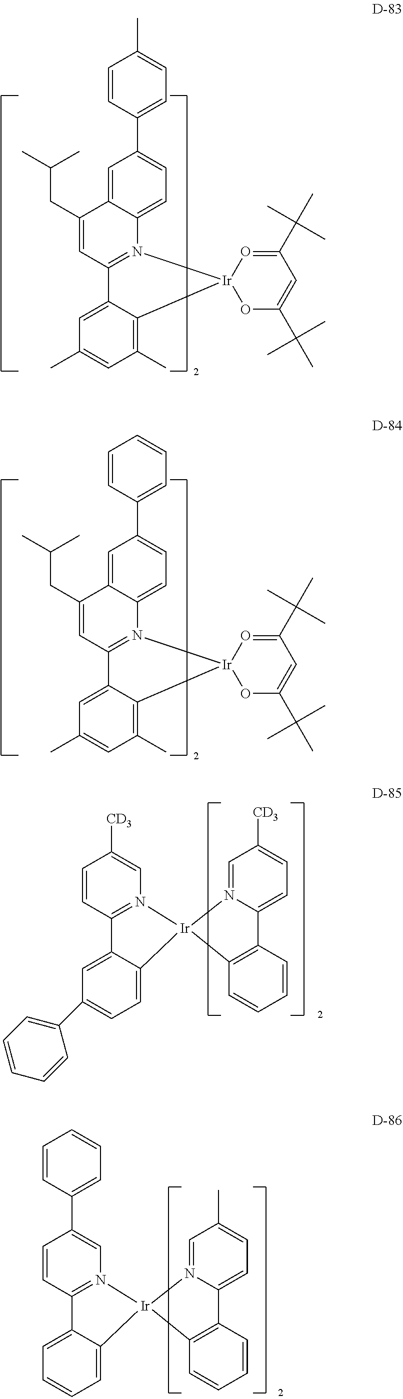

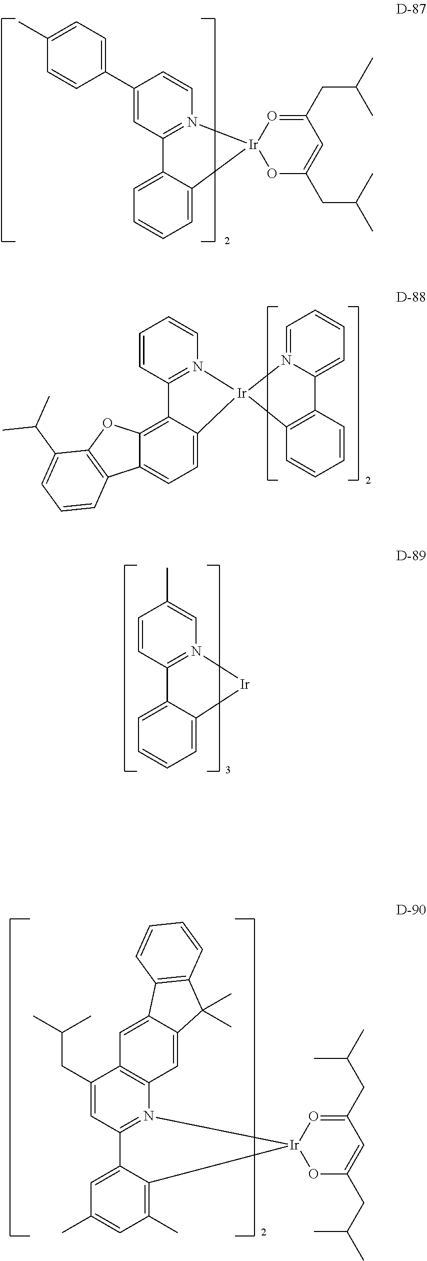

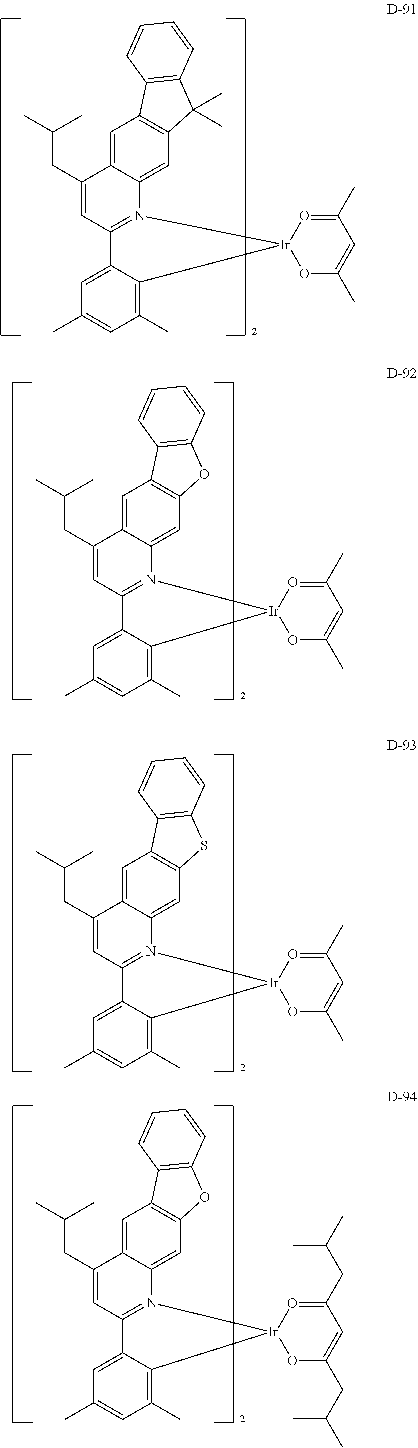

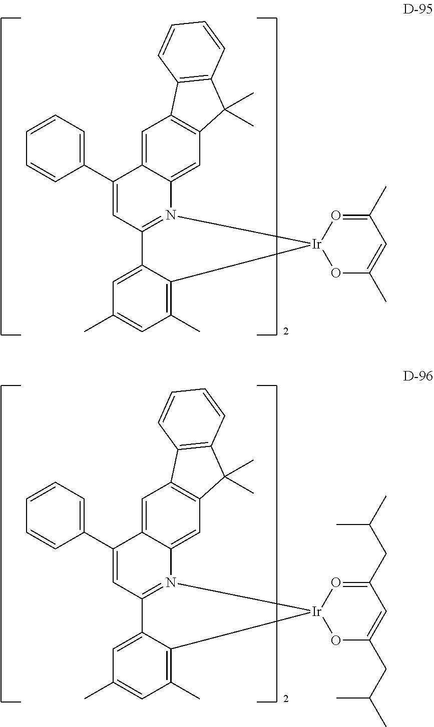

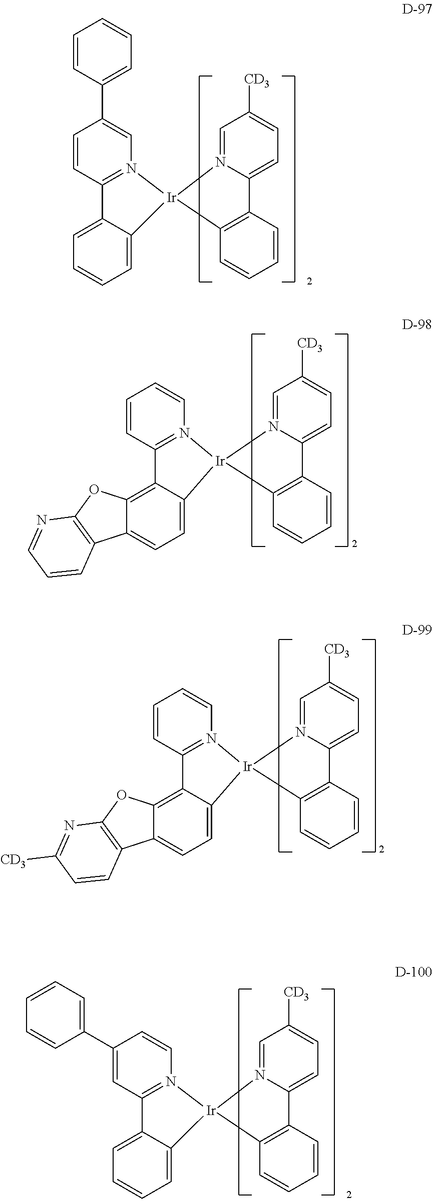

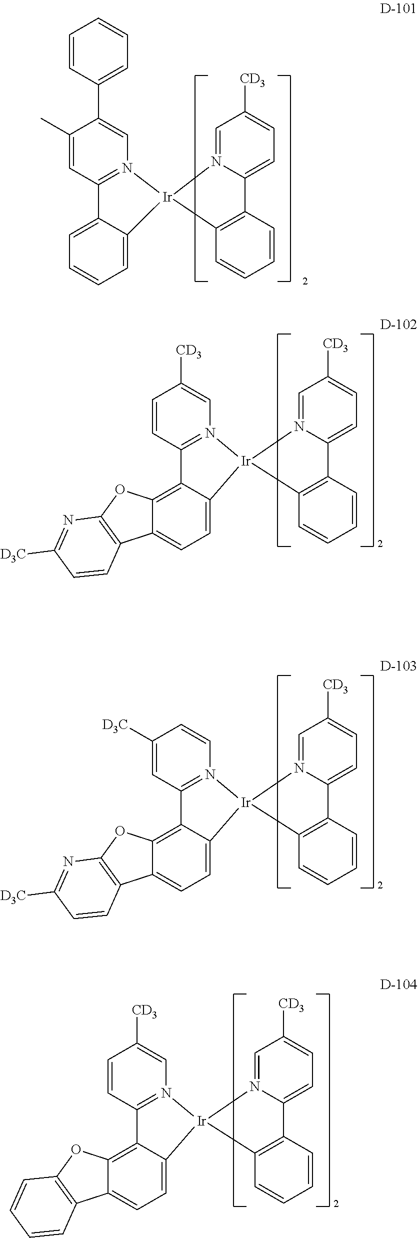

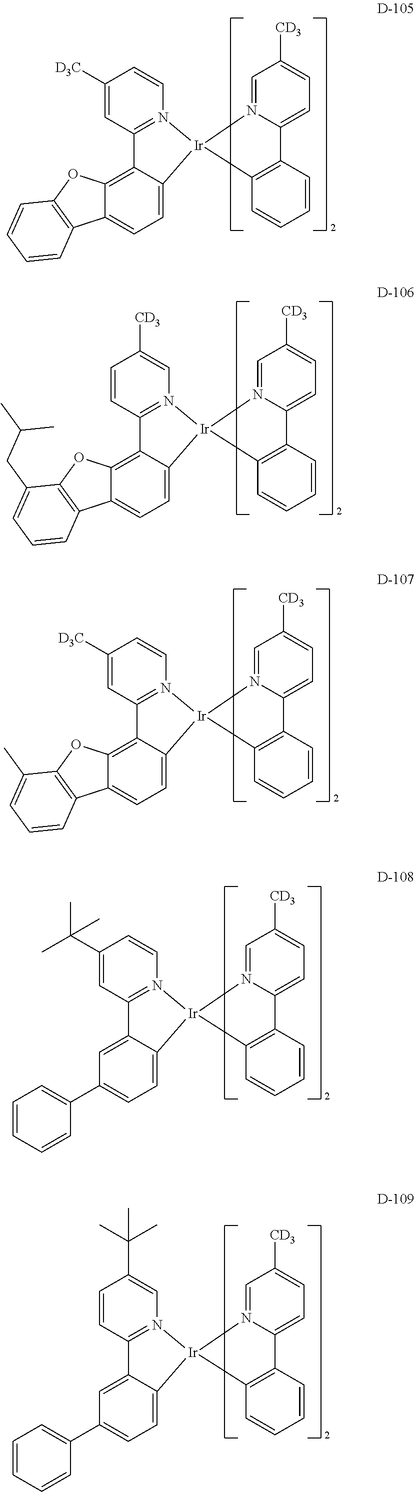

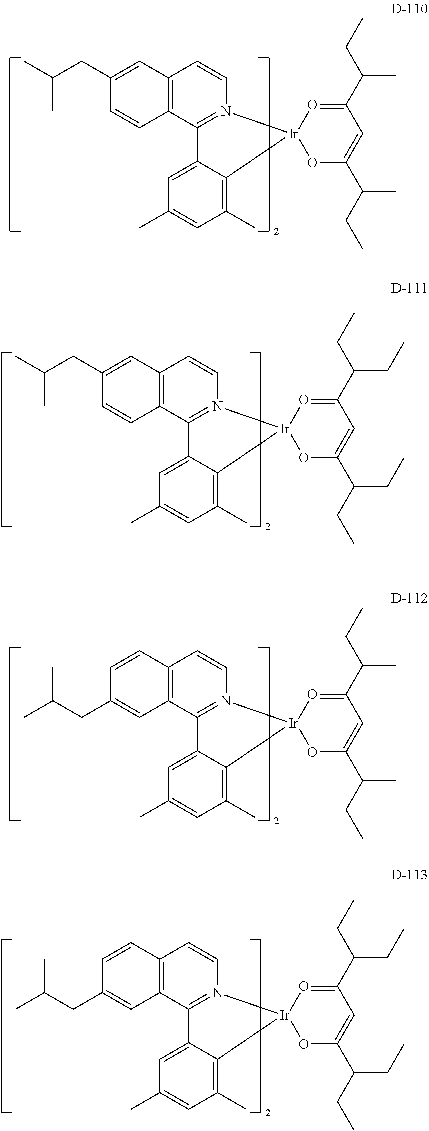

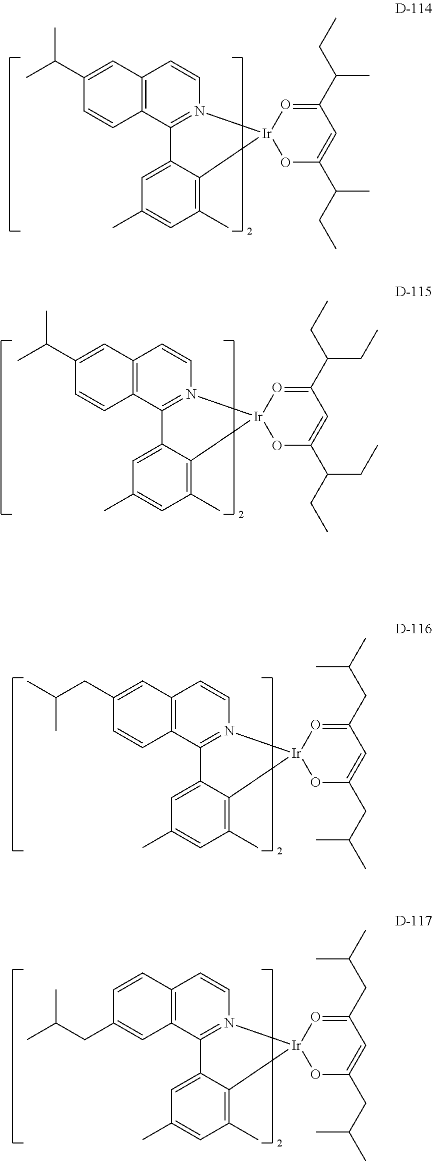

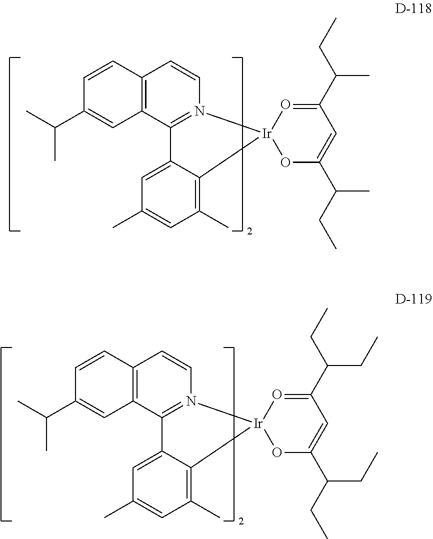

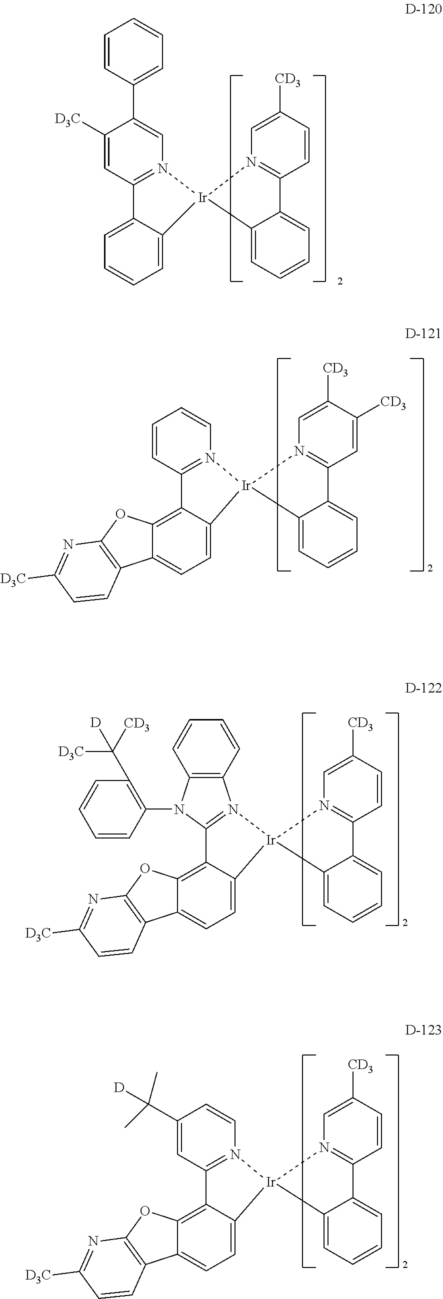

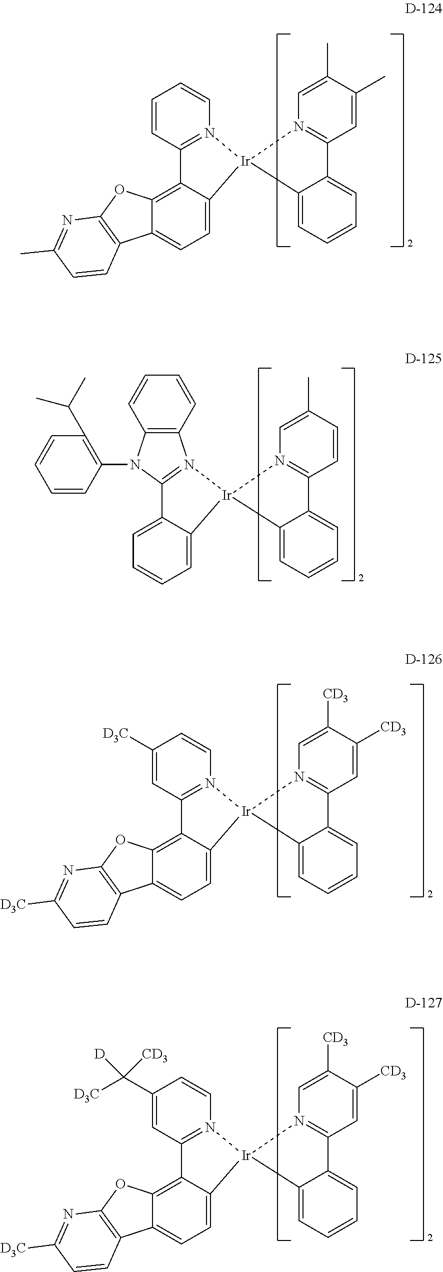

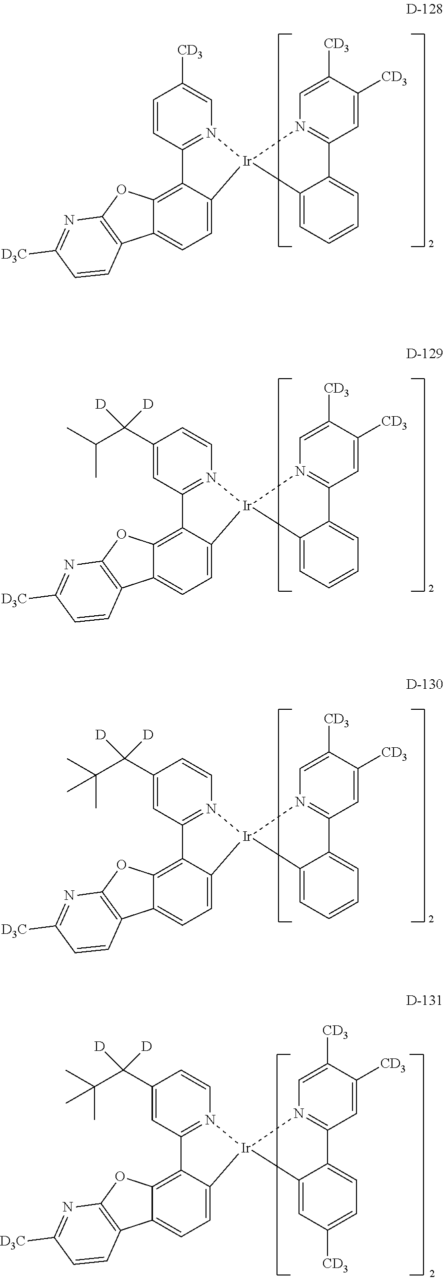

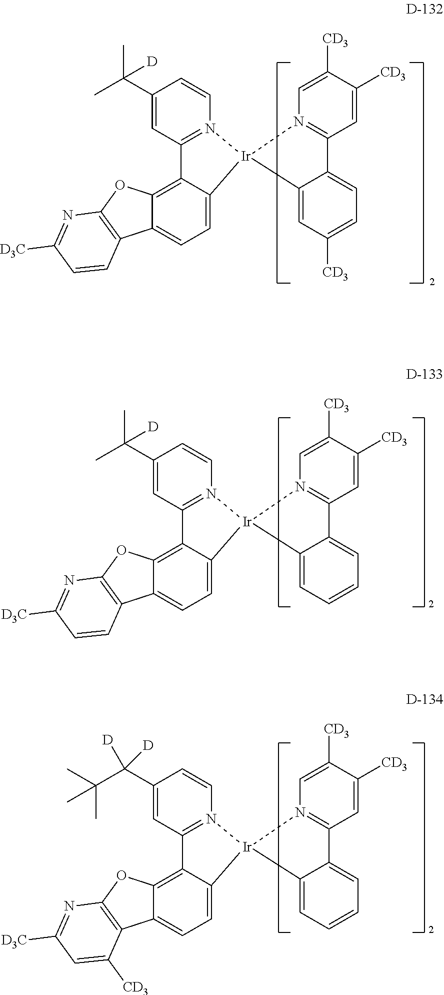

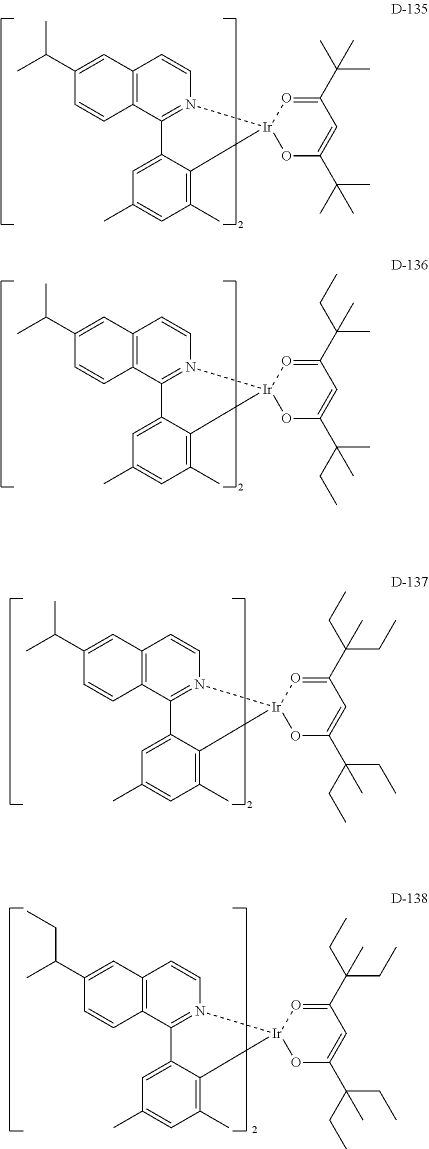

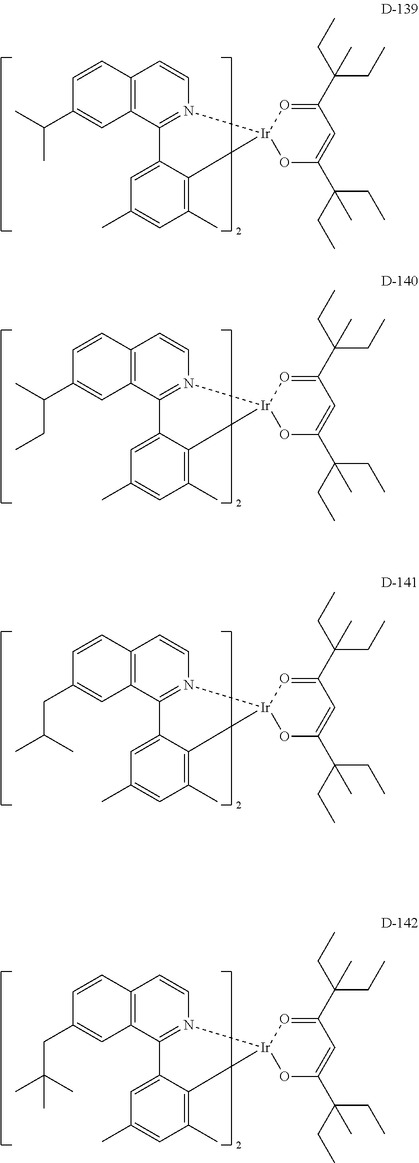

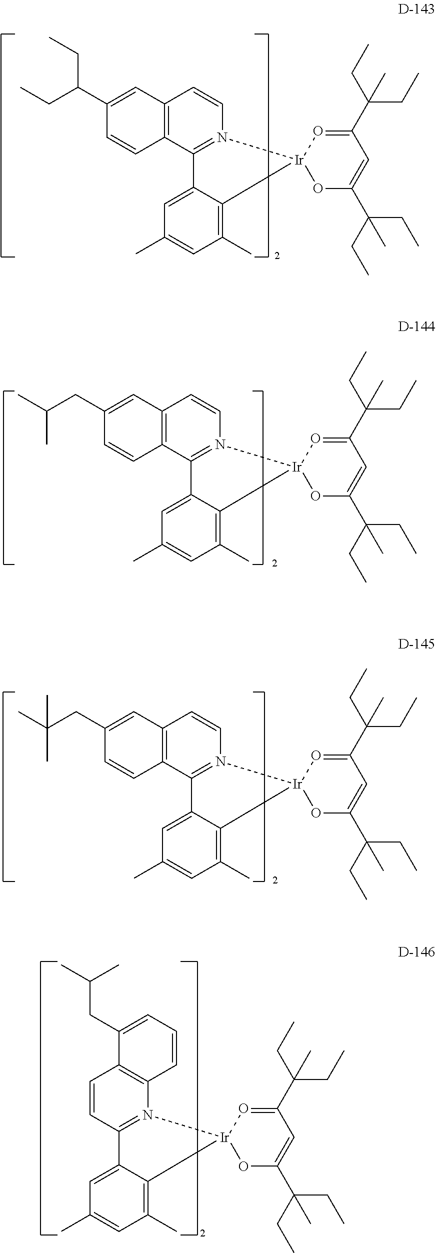

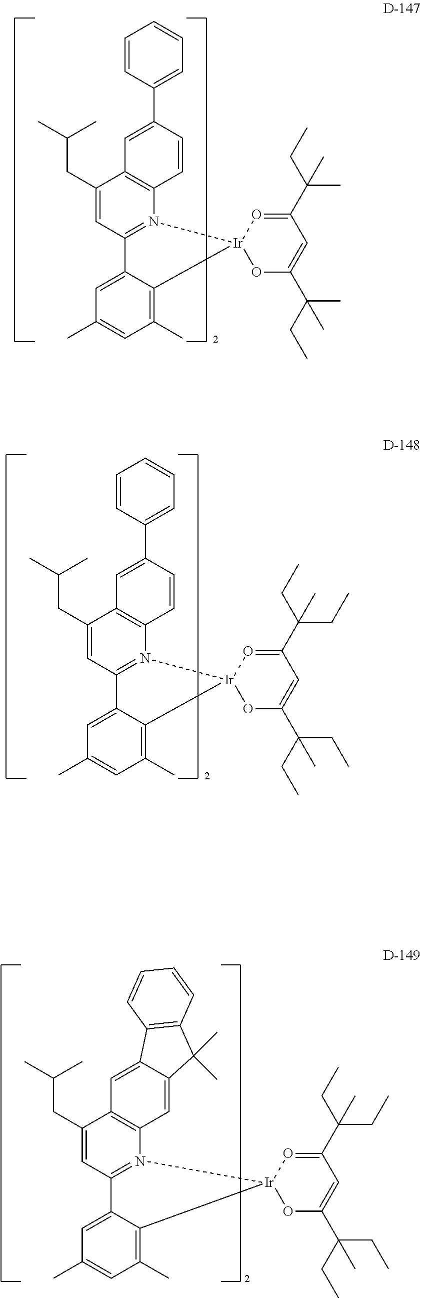

In a further aspect, the present disclosure provides a composition for manufacturing an organic electroluminescent device. The composition is preferably a composition for preparing a hole transport layer, a hole auxiliary layer, an electron blocking layer, a light-emitting layer, or a light-emitting auxiliary layer of an organic electroluminescent device and includes the compound of the present disclosure. If there are two or more hole transport layers, the compound of the present disclosure may be included in the composition for preparing a hole transport layer (hole auxiliary layer) adjacent to a light-emitting layer.

The organic electroluminescent device of the present disclosure comprises an anode; a cathode; and at least one organic layer between the anode and the cathode, wherein the organic layer may comprise a light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as a plurality of layers.

The anode and the cathode may be respectively formed with a transparent conductive material, or a transflective or reflective conductive material. The organic electroluminescent device may be a top emission type, a bottom emission type, or a both-sides emission type, depending on the materials forming the anode and the cathode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds.

Further, in the organic electroluminescent device of the present disclosure, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, a red, or a green electroluminescent compound known in the field, besides the compound of the present disclosure. If necessary, it may further comprise a yellow or an orange light-emitting layer.

In the organic electroluminescent device of the present disclosure, preferably, at least one layer selected from the group consisting of a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiOₓ (1≤X≤2), AlOₓ (1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

The organic electroluminescent material according to the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The organic electroluminescent material according to the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

The organic electroluminescent device according to one embodiment of the present disclosure may be an organic electroluminescent device having a tandem structure. In the case of a tandem organic electroluminescent device according to one embodiment, a single light-emitting unit (light-emitting part) may be formed in a structure in which two or more light-emitting units are connected by a charge generation layer. The organic electroluminescent device may comprise a plurality of two or more light-emitting units having first and second electrodes opposed to each other on a substrate and a light-emitting layer that is stacked between the first and second electrodes and emits light in a specific wavelength range, for example, three or more light-emitting units. The organic electroluminescent device may comprise a plurality of light-emitting units, and each light-emitting unit may comprise a hole transport zone, a light-emitting layer, and an electron transport zone. The hole transport zone may comprise a hole injection layer and a hole transport layer. The electron transport zone may include an electron transport layer and an electron injection layer. According to one embodiment, the light-emitting unit may include three or more light-emitting layers. A plurality of light-emitting units may emit the same color or different colors. Additionally, one light-emitting unit may comprise one or more light-emitting layers, and the plurality of light-emitting layers may be of the same or different colors. The organic electroluminescent device may comprise one or more charge generation layers located between each light-emitting unit. The charge generation layer refers to the layer in which holes and electrons are generated when voltage is applied. When there are three or more light-emitting units, a charge generation layer may be located between each light-emitting unit. In this case, the plurality of charge generation layers may be the same as or different from each other. By arranging the charge generation layer between light-emitting units, current efficiency may be increased in each light-emitting unit, and charges may be smoothly distributed. Specifically, the charge generation layer is provided between two adjacent stacks and can serve to drive a tandem organic electroluminescent device using only a pair of anode and cathode without a separate internal electrode located between the stacks.

The charge generation layer may be composed of an N-type charge generation layer and a P-type charge generation layer, and the N-type charge generation layer may be doped with an alkali metal, an alkaline earth metal, or a compound of an alkali metal and an alkaline earth metal. The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The P-type charge generation layer may be composed of a metal or an organic material doped with a P-type dopant. For example, the metal may be made of one or more alloys selected from the group consisting of Al, Cu, Fe, Pb, Zn, Au, Pt, W, In, Mo, Ni, and Ti. Additionally, commonly used materials may be used as the P-type dopant and host materials used in the P-type doped organic material.

The manufacturing method of the organic electroluminescent device of the present disclosure is not limited, and the manufacturing method of the Device Example as described below is only an example and is not limited thereto. One skilled in the art can reasonably modify the manufacturing method of the Device Examples as described below by relying on existing technology. For example, there is no particular limitation on the mixing ratio of the first compound and the second compound, and thus one skilled in the art can reasonably select it within a certain range by depending on existing technology. For example, based on the total weight of the light-emitting layer material, the total weight of the first compound and the second compound accounts for 99.5%-80.0% of the total weight of the light-emitting layer, the weight ratio of the first compound and the second compound is between 1:99 and 99:1, the weight ratio of the first compound and the second compound may be between 20:80 and 99:1, or the weight ratio of the first compound and the second compound may be between 50:50 and 90:10. In the manufacture of devices, when forming a light-emitting layer by co-depositing two or more host materials and a light-emitting material, the two or more host materials and the light-emitting material may be respectively placed in different evaporation sources and co-deposited to form a light-emitting layer, or a pre-mixed mixture of two or more host materials may be placed on the same evaporation source and then co-deposited with a light-emitting material placed on another evaporation source to form a light-emitting layer. This premixing method can further save evaporation sources. According to one embodiment, the first compound, the second compound, and the light-emitting material of the present disclosure may be respectively placed in different evaporation sources and co-deposited to form a light-emitting layer, or a pre-mixed mixture of the first compound and the second compound may be placed in the same evaporation source and then co-deposited with a light-emitting material placed in another evaporation source to form a light-emitting layer.

In addition, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the organic electroluminescent compounds according to the present disclosure and the properties thereof, and the luminous characteristics of an organic electroluminescent device (OLED) comprising an organic electroluminescent compound according to the present disclosure will be explained in detail with reference to the representative compounds of the present disclosure, but the present disclosure is not limited to the following examples.

### Example 1: Preparation of Compound C-1

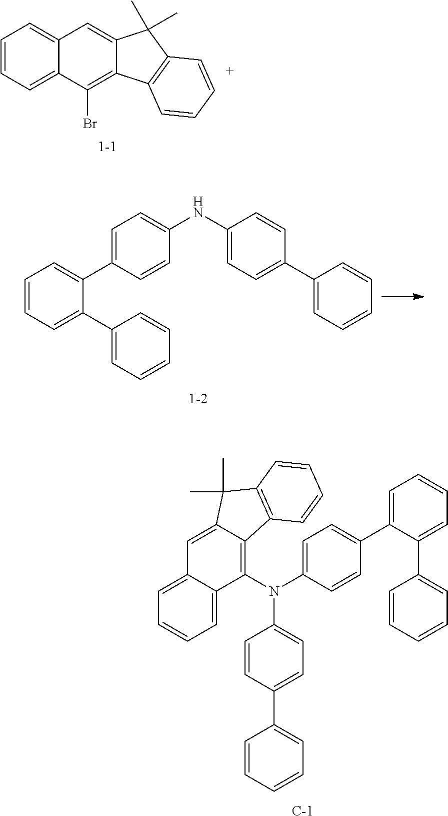

Compound 1-1 (10 g, 30.94 mmol), Compound 1-2 (13.53 g, 34.03 mmol), Pd₂(dba)₃ (1.42 g, 1.55 mmol), NaOtBu (5.95 g, 61.88 mmol), s-Phos (1.27 g, 3.094 mmol), and toluene (155 mL, 0.2 M) were added to a flask, dissolved, and refluxed at 150° C. for 2 hours. After completion of the reaction, an organic layer was extracted with ethyl acetate. After the residual moisture was removed using magnesium sulfate, the residue was dried and separated by column chromatography to obtain Compound C-1 (4.6 g, yield: 23%).

| | MW | M.P. |
|---|---|---|
| C-1 | 639.84 | 198.2° C. |

### Example 2: Preparation of Compound C-16

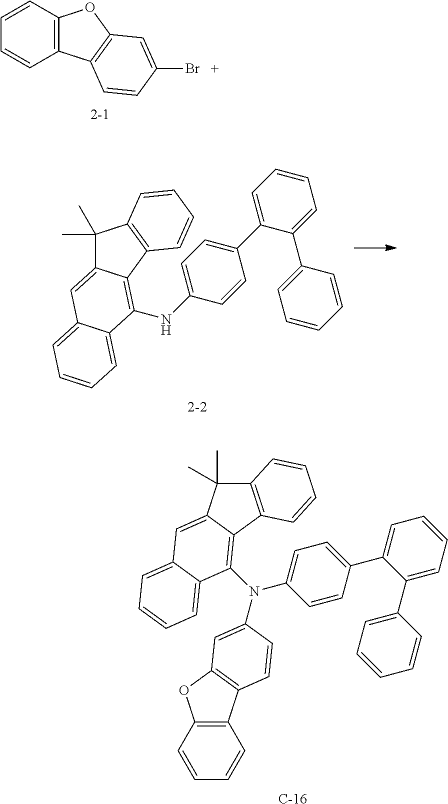

Compound 2-1 (10.7 g, 22 mmol), Compound 2-2 (6 g, 24 mmol), Pd₂(dba)₃ (1.1 g, 1.2 mmol), NaOtBu (7 g, 73 mmol), P(t-Bu)₃ (1.2 mL, 2.4 mmol), and toluene (120 mL, 0.2 M) were added to a flask, dissolved, and refluxed at 120° C. for 1 hour. After completion of the reaction, an organic layer was extracted with ethyl acetate. After the residual moisture was removed using magnesium sulfate, the residue was dried and separated by column chromatography to obtain Compound C-16 (8.4 g, yield: 52%).

| | MW | M.P. |
|---|---|---|
| C-16 | 653.83 | 173.6° C. |

### Example 3: Preparation of Compound C-13

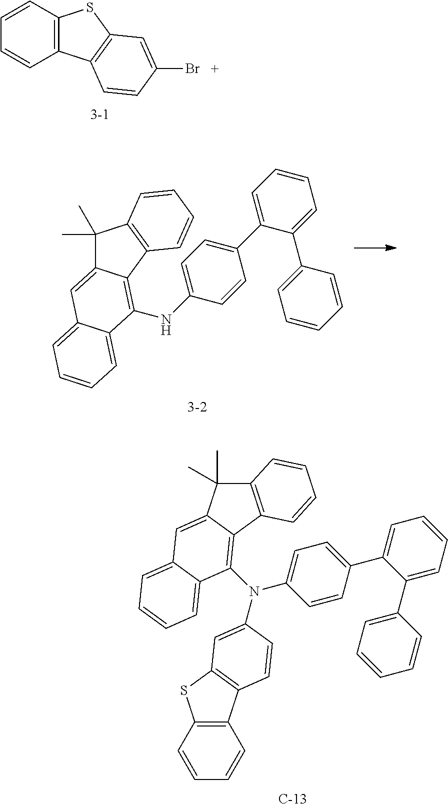

Compound 3-1 (10 g, 21 mmol), Compound 3-2 (6 g, 23 mmol), Pd₂(dba)₃ (1 g, 1.1 mmol), NaOtBu (6.6 g, 68 mmol), P(t-Bu)₃ (1.1 mL, 2.2 mmol), and toluene (115 mL, 0.2 M) were added to a flask, dissolved, and refluxed at 120° C. for 1 hour. After completion of the reaction, an organic layer was extracted with ethyl acetate. After the residual moisture was removed using magnesium sulfate, the residue was dried and separated by column chromatography to obtain Compound C-13 (5.8 g, yield: 38%).

| | MW | M.P. |
|---|---|---|
| C-13 | 669.89 | 164.2° C. |

### Example 4: Preparation of Compound C-2

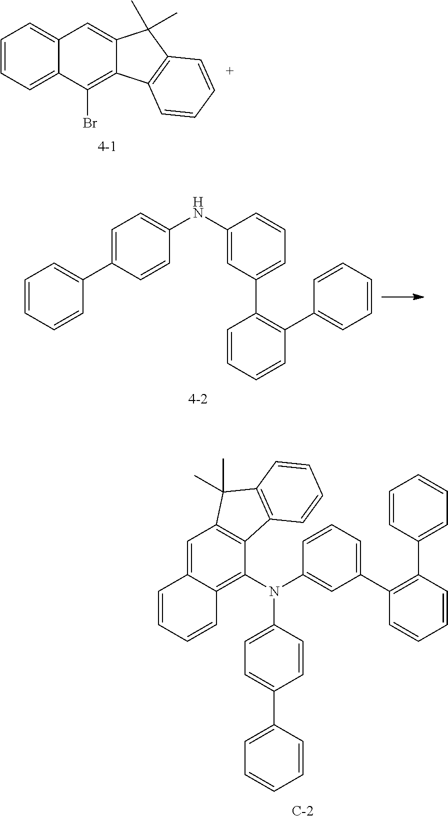

Compound 4-1 (10 g, 30.94 mmol), Compound 4-2 (12.30 g, 30.94 mmol), Pd₂(dba)₃ (1.42 g, 1.55 mmol), NaOtBu (5.95 g, 61.88 mmol), s-Phos (1.27 g, 3.094 mmol), and toluene (155 mL, 0.2 M) were added to a flask, dissolved, and refluxed at 150° C. for 2 hours. After completion of the reaction, an organic layer was extracted with ethyl acetate. After the residual moisture was removed using magnesium sulfate, the residue was dried and separated by column chromatography to obtain Compound C-2 (3.1 g, yield: 16%).

| | MW | M.P. |
|---|---|---|
| C-2 | 639.84 | 134° C. |

### Example 5: Preparation of Compound C-7

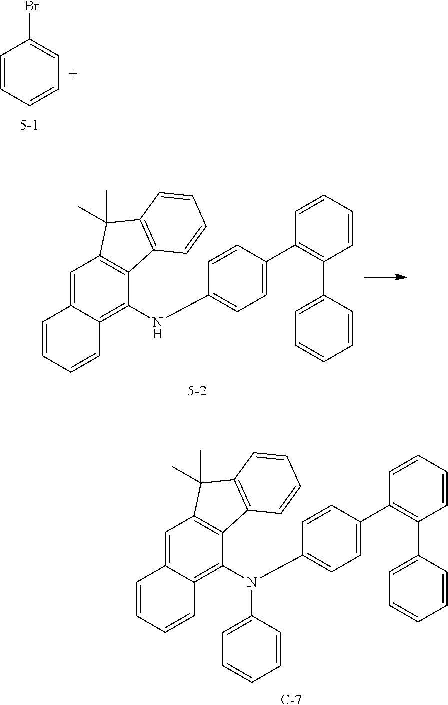

Compound 5-1 (3.94 g, 25.07 mmol), Compound 5-2 (11 g, 22.56 mmol), Pd₂(dba)₃ (1.15 g, 1.254 mmol), NaOtBu (7.23 g, 75.21 mmol), P(t-Bu)₃ (507 mg, 2.51 mmol), and toluene (125 mL, 0.2 M) were added to a flask, dissolved, and refluxed at 120° C. for 12 hours. After completion of the reaction, an organic layer was extracted with ethyl acetate. After the residual moisture was removed using magnesium sulfate, the residue was dried and separated by column chromatography to obtain Compound C-7 (7.8 g, yield: 61%).

| | MW | M.P. |
|---|---|---|
| C-7 | 563.74 | 246° C. |

### Example 6: Preparation of Compound C-8

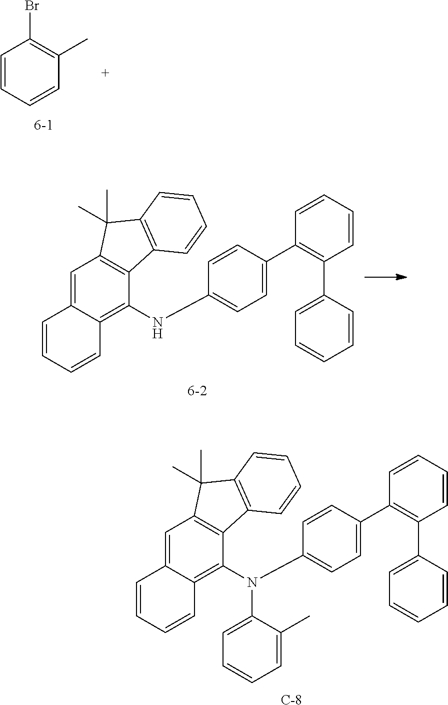

Compound 6-1 (7.72 g, 45.11 mmol), Compound 6-2 (11 g, 22.56 mmol), Pd₂(dba)₃ (1.03 g, 1.128 mmol), NaOtBu (6.5 g, 67.68 mmol), P(t-Bu)₃ (456 mg, 2.256 mmol), and toluene (113 mL, 0.2 M) were added to a flask, dissolved, and refluxed at 120° C. for 12 hours. After completion of the reaction, an organic layer was extracted with ethyl acetate. After the residual moisture was removed using magnesium sulfate, the residue was dried and separated by column chromatography to obtain Compound C-8 (11.4 g, yield: 87%).

| | MW | M.P. |
|---|---|---|
| C-8 | 577.97 | 193° C. |

### Example 7: Preparation of Compound C-3

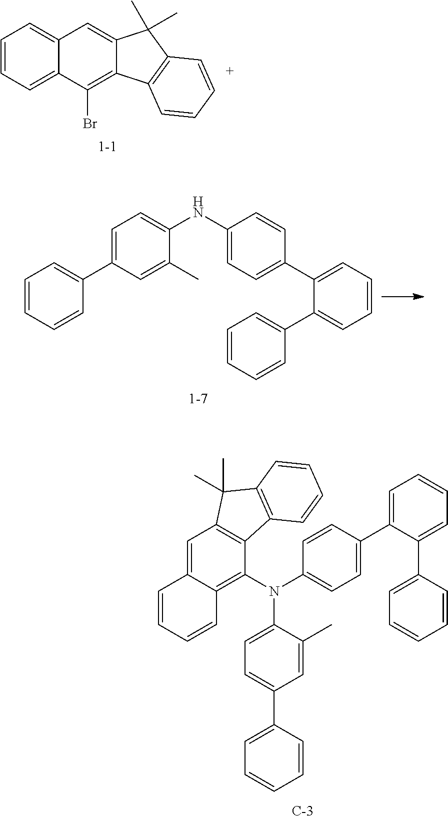

Compound 1-1 (10 g, 30.94 mmol), compound 1-7 (11.46 g, 27.84 mmol), Pd₂(dba)₃ (1.42 g, 1.55 mmol), NaOtBu (5.95 g, 61.88 mmol), P(t-Bu)₃ (626 mg, 3.094 mmol), and toluene (155 mL, 0.2 M) were added to a flask, dissolved, and refluxed at 150° C. for 2 hours. After completion of the reaction, an organic layer was extracted with ethyl acetate. After the residual moisture was removed using magnesium sulfate, the residue was dried and separated by column chromatography to obtain Compound C-3 (10.3 g, yield: 51%).

| | MW | M.P. |
|---|---|---|
| C-3 | 653.87 | 199° C. |

### Example 8: Preparation of Compound C-191

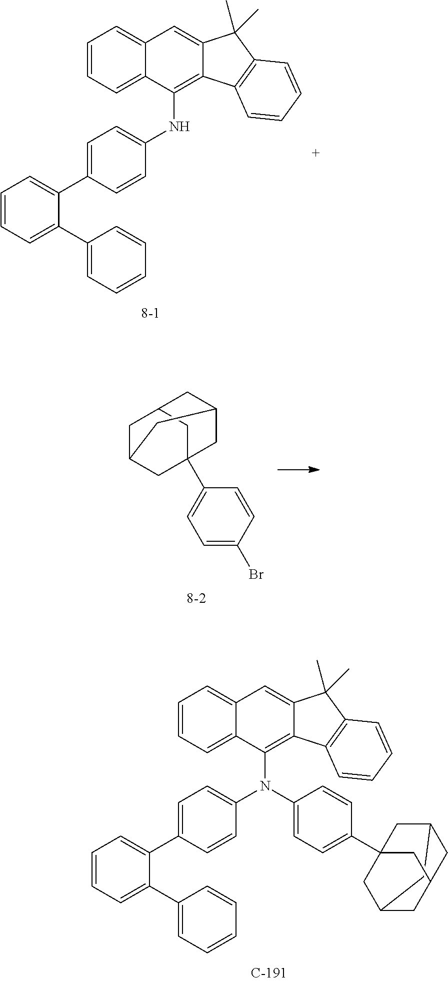 [SMILES: CC1(C)c2ccccc2-c2c1cc1ccccc1c2Nc1ccc(-c2ccccc2-c2ccccc2)cc1 ; Brc1ccc(C23CC4CC(CC(C4)C2)C3)cc1 ; CC1(C)c2ccccc2-c2c1cc1ccccc1c2N(c1ccc(-c2ccccc2-c2ccccc2)cc1)c1ccc(C23CC4CC(CC(C4)C2)C3)cc1]

Compound 8-1 (8 g, 16.0 mmol), Compound 8-2 (5.10 g, 17.7 mmol), Pd₂(dba)₃ (0.82 g, 0.09 mmol), NaOtBu (3.4 g, 35.40 mmol), s-Phos (2.0 g, 0.18 mmol), and toluene (90 mL, 0.2 M) were added to a flask, dissolved, and refluxed at 150° C. for 2 hours. After completion of the reaction, an organic layer was extracted with ethyl acetate. After the residual moisture was removed using magnesium sulfate, the residue was dried and separated by column chromatography to obtain Compound C-191 (7.1 g, yield: 57%).

| | MW | M.P. |
|---|---|---|
| C-191 | 697.97 | 340° C. |

### Device Examples 1 to 7: Producing OLEDs Comprising an Organic Electroluminescent Compound According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and Compound HT-1 was introduced into another cell of the vacuum vapor deposition apparatus. The two materials were evaporated at different rates, and Compound HI-1 was deposited in a doping amount of 3 wt % based on the total amount of Compound HI-1 and Compound HT-1 to form a hole injection layer having a thickness of 10 nm on the ITO substrate. Next, Compound HT-1 was deposited on the hole injection layer to form a first hole transport layer having a thickness of 90 nm. The compound shown in Table 1 below was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: Compound PRH was introduced into two cells of the vacuum vapor deposition apparatus as hosts, and Compound D-39 was introduced into another cell as a dopant. The two materials were evaporated at different rates and the dopant was deposited in a doping amount of 2 wt % based on the total amount of the host and the dopant to form a light-emitting layer having a thickness of 40 nm on the second hole transport layer. Next, compound HBL was deposited to a thickness of 5 nm as an electron buffer layer on the light-emitting layer. Compound ETL-1 and Compound EIL-1 were evaporated in a weight ratio of 50:50 to form an electron transport layer having a thickness of 30 nm on the electron buffer layer. After depositing Compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Examples 1 to 3: Producing an OLED not Comprising an Organic Electroluminescent Compound According to the Present Disclosure

An OLED was produced in the same manner as in Device Examples 1 to 7, except that the compound shown in Table 1 below was used as a material of the second hole transport layer.

The driving voltage, luminous efficiency, and light-emitting color at a luminance of 1,000 nit, and the time taken for luminance to decrease from 100% to 95% at a luminance of 10,000 nit (lifetime; T95) of the OLEDs produced in Device Examples 1 to 7 and Comparative Examples 1 to 3 are provided in Table 1 below.

TABLE 1

| | Second | | | | Life- |
|---|---|---|---|---|---|
| | Hole | Driving | Luminous | Light- | time |
| | Transport | Voltage | Efficiency | Emitting | T95 |
| | Layer | (V) | (lm/W) | Color | (hr) |
| Device Example 1 | C-1 | 3.0 | 30.3 | Red | 163.5 |
| Device Example 2 | C-13 | 3.0 | 26.8 | Red | 139.1 |
| Device Example 3 | C-16 | 3.0 | 30.5 | Red | 156.3 |
| Device Example 4 | C-2 | 3.7 | 23.8 | Red | 183.8 |
| Device Example 5 | C-7 | 3.5 | 27.2 | Red | 195.1 |
| Device Example 6 | C-8 | 3.9 | 24.4 | Red | 223.1 |
| Device Example 7 | C-191 | 3.3 | 28.9 | Red | 155.7 |
| Comparative | Ref-1 | 2.9 | 29.3 | Red | 40.7 |
| Example 1 | | | | | |
| Comparative | Ref-2 | 3.0 | 24.9 | Red | 33.8 |
| Example 2 | | | | | |
| Comparative | Ref-3 | 4.2 | 21.4 | Red | 68.2 |
| Example 3 | | | | | |

As shown in Table 1 above, it can be confirmed that the OLEDs comprising the compound according to the present disclosure in the second hole transport layer exhibit a driving voltage and luminous efficiency characteristics in equivalent level, and significantly improved lifetime properties, compared to the OLED not comprising the compound according to the present disclosure.

### [Table 2]

The compounds used in the Device Examples and the Comparative Examples are shown in Table 2 below.

| | |
|---|---|
| Hole Injection Layer/ First Hole Transport Layer | 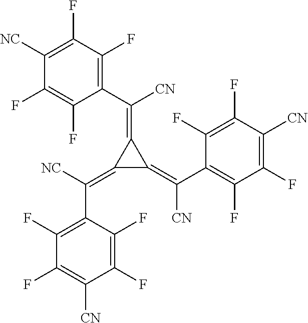 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 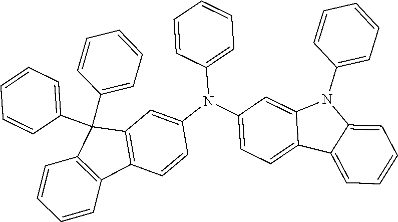 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-1 |
| Second Hole Transport Layer | 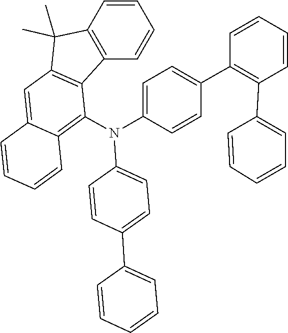 [SMILES: CC1(C)c2ccccc2-c2c1cc1ccccc1c2N(c1ccc(-c2ccccc2)cc1)c1ccc(-c2ccccc2-c2ccccc2)cc1] |
| | C-1 |
| | 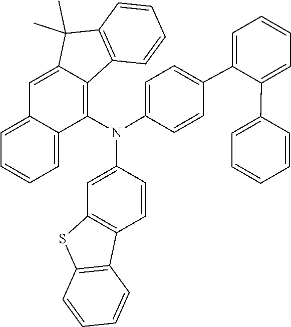 [SMILES: CC1(C)c2ccccc2-c2c1cc1ccccc1c2N(c1ccc(-c2ccccc2-c2ccccc2)cc1)c1ccc2c(c1)sc1ccccc12] |
| | C-13 |
| | 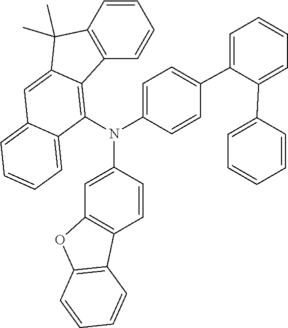 [SMILES: CC1(C)c2ccccc2-c2c1cc1ccccc1c2N(c1ccc(-c2ccccc2-c2ccccc2)cc1)c1ccc2c(c1)oc1ccccc12] |
| | C-16 |
| | 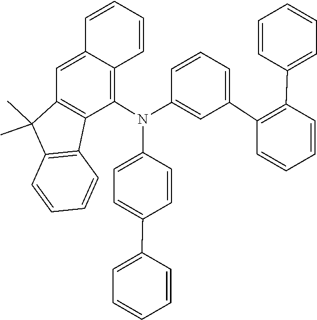 [SMILES: CC1(C)c2ccccc2-c2c1cc1ccccc1c2N(c1ccc(-c2ccccc2)cc1)c1cccc(-c2ccccc2-c2ccccc2)c1] |
| | C-2 |
| | 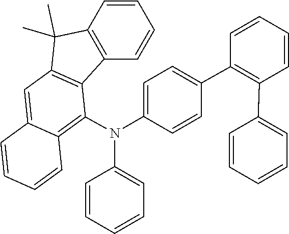 [SMILES: CC1(C)c2ccccc2-c2c1cc1ccccc1c2N(c1ccccc1)c1ccc(-c2ccccc2-c2ccccc2)cc1] |
| | C-7 |
| | 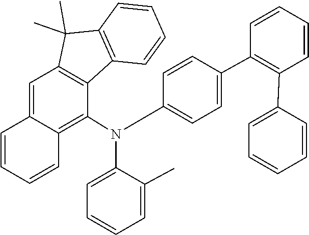 [SMILES: Cc1ccccc1N(c1ccc(-c2ccccc2-c2ccccc2)cc1)c1c2c(cc3ccccc13)C(C)(C)c1ccccc1-2] |
| | C-8 |
| | 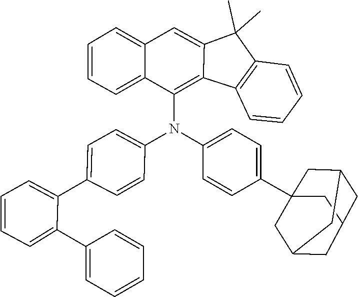 [SMILES: CC1(C)c2ccccc2-c2c1cc1ccccc1c2N(c1ccc(-c2ccccc2-c2ccccc2)cc1)c1ccc(C23CC4CC(CC(C4)C2)C3)cc1] |
| | C-191 |
| | 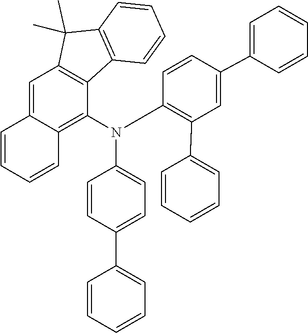 [SMILES: CC1(C)c2ccccc2-c2c1cc1ccccc1c2N(c1ccc(-c2ccccc2)cc1)c1ccc(-c2ccccc2)cc1-c1ccccc1] |
| | Ref-1 |
| | 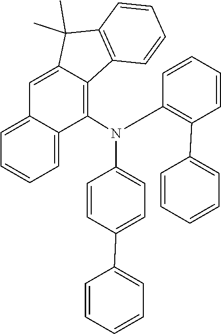 [SMILES: CC1(C)c2ccccc2-c2c1cc1ccccc1c2N(c1ccc(-c2ccccc2)cc1)c1ccccc1-c1ccccc1] |
| | Ref-2 |
| | 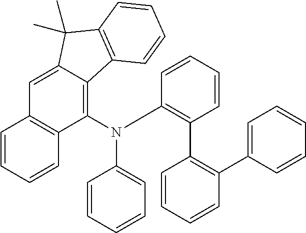 [SMILES: CC1(C)c2ccccc2-c2c1cc1ccccc1c2N(c1ccccc1)c1ccccc1-c1ccccc1-c1ccccc1] |
| | Ref-3 |
| Light-Emitting Layer | 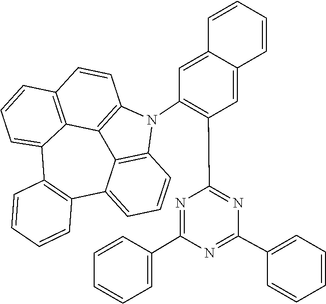 [SMILES: c1ccc(-c2nc(-c3ccccc3)nc(-c3cc4ccccc4cc3-n3c4cccc5c4c4c6c(cccc6ccc43)-c3ccccc3-5)n2)cc1] |
| | PRH |
| | 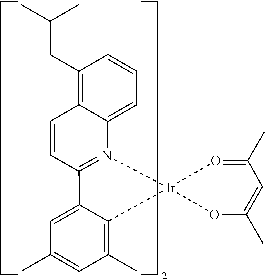 |
| | D-39 |
| Electron Buffer Layer | 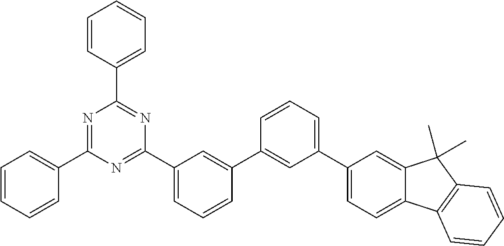 [SMILES: CC1(C)c2ccccc2-c2ccc(-c3cccc(-c4cccc(-c5nc(-c6ccccc6)nc(-c6ccccc6)n5)c4)c3)cc21] |
| | HBL |
| Electron Transport Layer/ Electron Injection Layer | 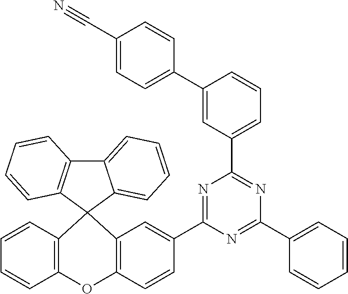 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | ETL-1 |
| | 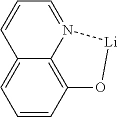 |
| | EIL-1 |

1. An organic electroluminescent compound represented by the following formula 1: 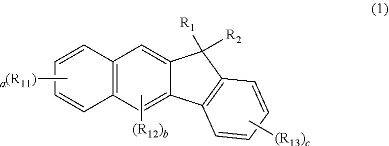
in formula 1,
R₁ and R₂, each independently, represent a substituted or unsubstituted (C1-C30)alkyl, a substituted or unsubstituted (C6-C30)aryl, or a substituted or unsubstituted (3- to 30-membered)heteroaryl; or may be linked to each other to form a ring(s);
R₁₁ to R₁₃, each independently, represent hydrogen, deuterium, or a substituted or unsubstituted (C6-C30)aryl, or are represented by the following formula 1-1, with the proviso that at least one of R₁₂ and R₁₃ is represented by the following formula 1-1; 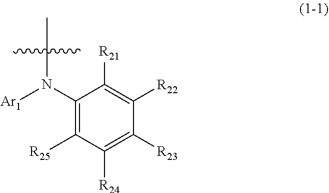
in formula 1-1,
Ar₁ represents a substituted or unsubstituted (C6-C30)aryl, or a substituted or unsubstituted (3- to 30-membered)heteroaryl;
R₂₁ and R₂₅, each independently, represent hydrogen, deuterium, an unsubstituted (C1-C30)alkyl, or a combination thereof;
R₂₂ to R₂₄, each independently, represent hydrogen, deuterium, a substituted or unsubstituted (C1-C30)alkyl, a substituted or unsubstituted (C6-C30)aryl, or a substituted or unsubstituted (3- to 30-membered)heteroaryl, with the proviso that at least one of R₂₂ to R₂₄ represents a substituted or unsubstituted (C12-C30)aryl comprising the following linking group a in ortho form; 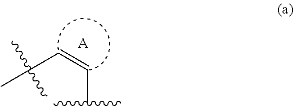
ring A represents a substituted or unsubstituted (C6-C24)arene ring;
a and c, each independently, represent an integer of 1 to 4, and b represents an integer of 1 to 2, in which if a to c represent an integer of 2 or more, each of R₁₁ to each of R₁₃ may be the same or different; and
the heteroaryl and heteroarene each comprise at least one heteroatoms selected from B, N, O, S, Si and P.

2. The organic electroluminescent compound according to claim 1, wherein the substituted aryl, the substituted arene, the substituted heteroaryl, the substituted heteroarene, and the substituted alkyl, each independently, are substituted with at least one selected from the group consisting of deuterium, a halogen, a cyano, a carboxyl, a nitro, a hydroxy, a phosphine oxide, a (C1-C30)alkyl unsubstituted or substituted with deuterium, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (3- to 30-membered)heteroaryl unsubstituted or substituted with a (C6-C30)aryl(s), a (C6-C30)aryl unsubstituted or substituted with a (3- to 30-membered)heteroaryl(s), a tri(C1-C30)alkylsilyl, a tri(C6-C30)arylsilyl, a di(C1-C30)alkyl(C6-C30)arylsilyl, a (C1-C30)alkyldi(C6-C30)arylsilyl, a fused ring group of a (C3-C30)aliphatic ring(s) and a (C6-C30)aromatic ring(s), an amino, a mono- or di-(C1-C30)alkylamino, a mono- or di-(C2-C30)alkenylamino, a mono- or di-(C6-C30)arylamino, a mono- or di-(3- to 30-membered)heteroarylamino, a (C1-C30)alkyl(C2-C30)alkenylamino, a (C1-C30)alkyl(C6-C30)arylamino, a (C1-C30)alkyl(3- to 30-membered)heteroarylamino, a (C2-C30)alkenyl(C6-C30)arylamino, a (C2-C30)alkenyl(3- to 30-membered)heteroarylamino, a (C6-C30)aryl(3- to 30-membered)heteroarylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

3. The organic electroluminescent compound according to claim 1, wherein formula 1 is represented by any one of the following formulas 2 to 4: 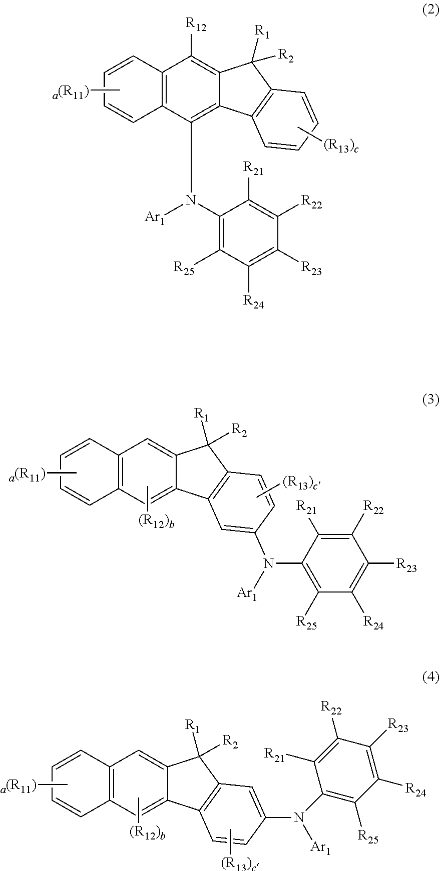
in formulas 2 to 4,
c′ represents an integer of 1 to 3, in which if c′ represents an integer of 2 or more, each of R₁₃ may be the same or different; and
R₁, R₂, R₁₁ to R₁₃, R₂₁ to R₂₅, Ar₁, a, and b are as defined in claim 1.

4. The organic electroluminescent compound according to claim 1, wherein the linking group a is represented by any one of the following structures: 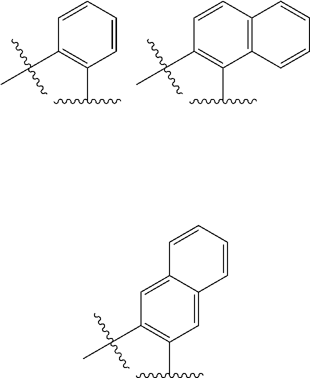 [SMILES: *c1ccccc1* ; *c1ccc2ccccc2c1* ; *c1cc2ccccc2cc1*]
in the structures, hydrogen may be replaced by at least one deuterium.

5. The organic electroluminescent compound according to claim 1, wherein the substituted or unsubstituted (C12-C30)aryl comprising the linking group a in ortho form is represented by any one of the following structures: 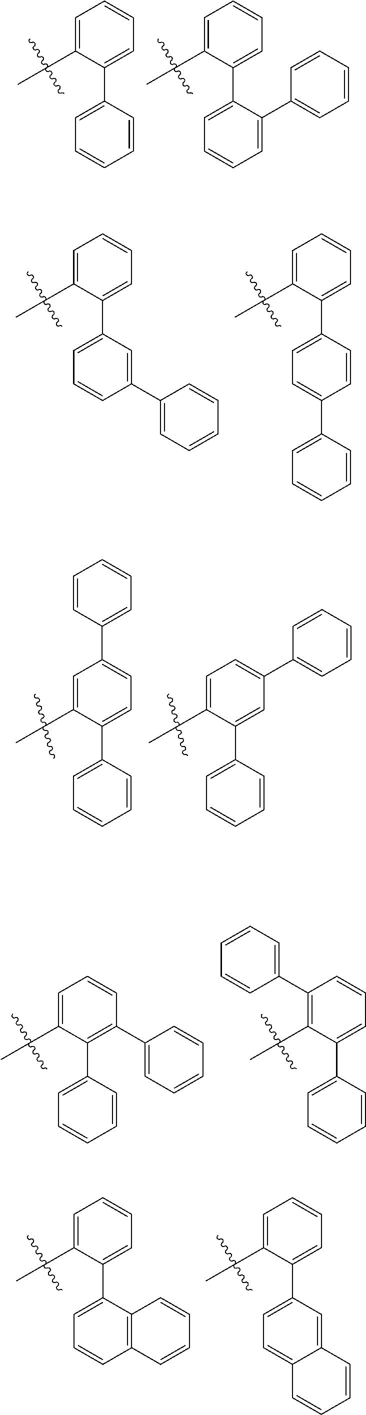 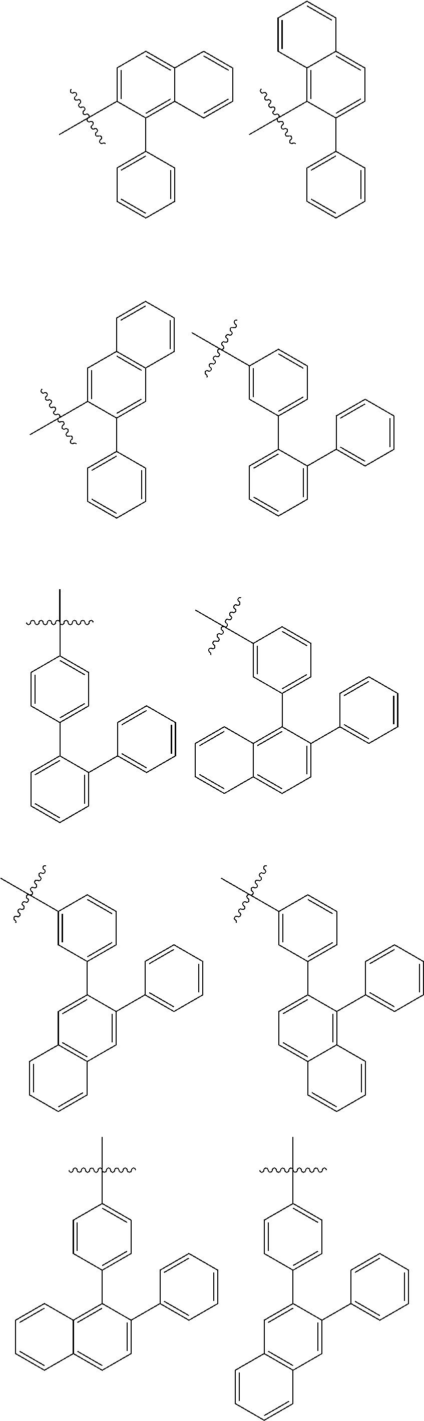 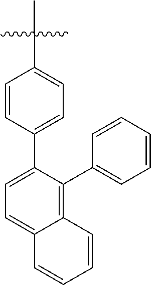 [SMILES: *c1ccc(-c2ccc3ccccc3c2-c2ccccc2)cc1]
in the structures, hydrogen may be replaced by at least one of deuterium and a methyl(s) unsubstituted or substituted with at least one deuterium.

6. The organic electroluminescent compound according to claim 1, wherein R₁ and R₂ each independently, represent a methyl unsubstituted or substituted with deuterium, or a phenyl unsubstituted or substituted with deuterium.

7. The organic electroluminescent compound according to claim 1, wherein R₁₁ to R₁₃ each independently, represent hydrogen, a methyl unsubstituted or substituted with deuterium, a phenyl unsubstituted or substituted with deuterium, or formula 1-1.

8. The organic electroluminescent compound according to claim 1, wherein the compound represented by formula 1 is selected from the following compounds: 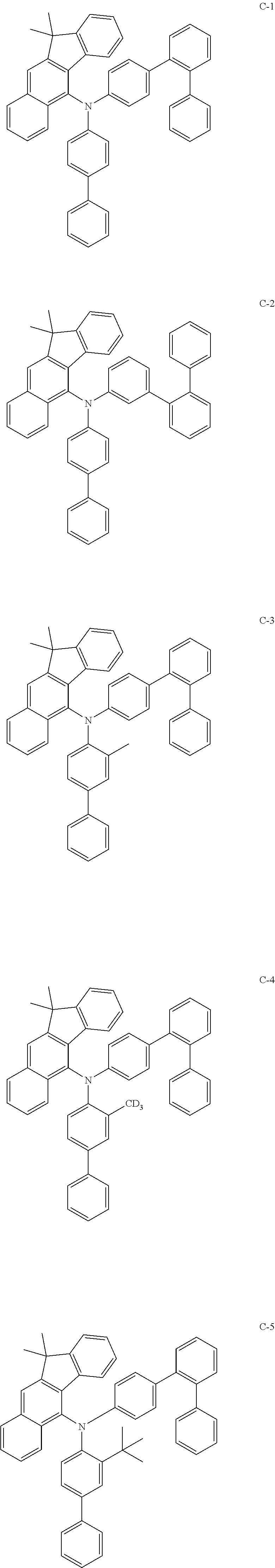 [SMILES: CC1(C)c2ccccc2-c2c1cc1ccccc1c2N(c1ccc(-c2ccccc2)cc1)c1ccc(-c2ccccc2-c2ccccc2)cc1 ; CC1(C)c2ccccc2-c2c1cc1ccccc1c2N(c1ccc(-c2ccccc2)cc1)c1cccc(-c2ccccc2-c2ccccc2)c1 ; Cc1cc(-c2ccccc2)ccc1N(c1ccc(-c2ccccc2-c2ccccc2)cc1)c1c2c(cc3ccccc13)C(C)(C)c1ccccc1-2 ; [2H]C([2H])([2H])c1cc(-c2ccccc2)ccc1N(c1ccc(-c2ccccc2-c2ccccc2)cc1)c1c2c(cc3ccccc13)C(C)(C)c1ccccc1-2 ; CC(C)(C)c1cc(-c2ccccc2)ccc1N(c1ccc(-c2ccccc2-c2ccccc2)cc1)c1c2c(cc3ccccc13)C(C)(C)c1ccccc1-2] 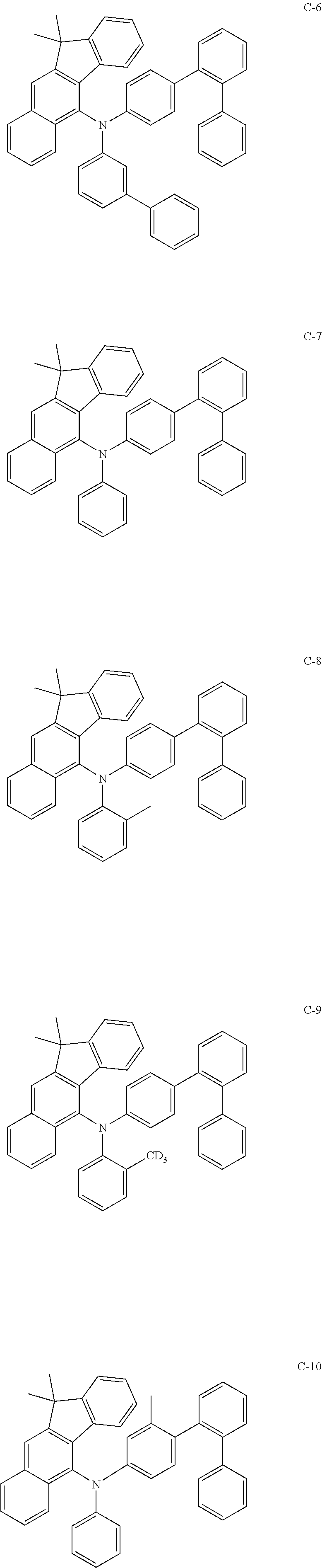 [SMILES: CC1(C)c2ccccc2-c2c1cc1ccccc1c2N(c1ccc(-c2ccccc2-c2ccccc2)cc1)c1cccc(-c2ccccc2)c1 ; CC1(C)c2ccccc2-c2c1cc1ccccc1c2N(c1ccccc1)c1ccc(-c2ccccc2-c2ccccc2)cc1 ; Cc1ccccc1N(c1ccc(-c2ccccc2-c2ccccc2)cc1)c1c2c(cc3ccccc13)C(C)(C)c1ccccc1-2 ; [2H]C([2H])([2H])c1ccccc1N(c1ccc(-c2ccccc2-c2ccccc2)cc1)c1c2c(cc3ccccc13)C(C)(C)c1ccccc1-2 ; Cc1cc(N(c2ccccc2)c2c3c(cc4ccccc24)C(C)(C)c2ccccc2-3)ccc1-c1ccccc1-c1ccccc1] 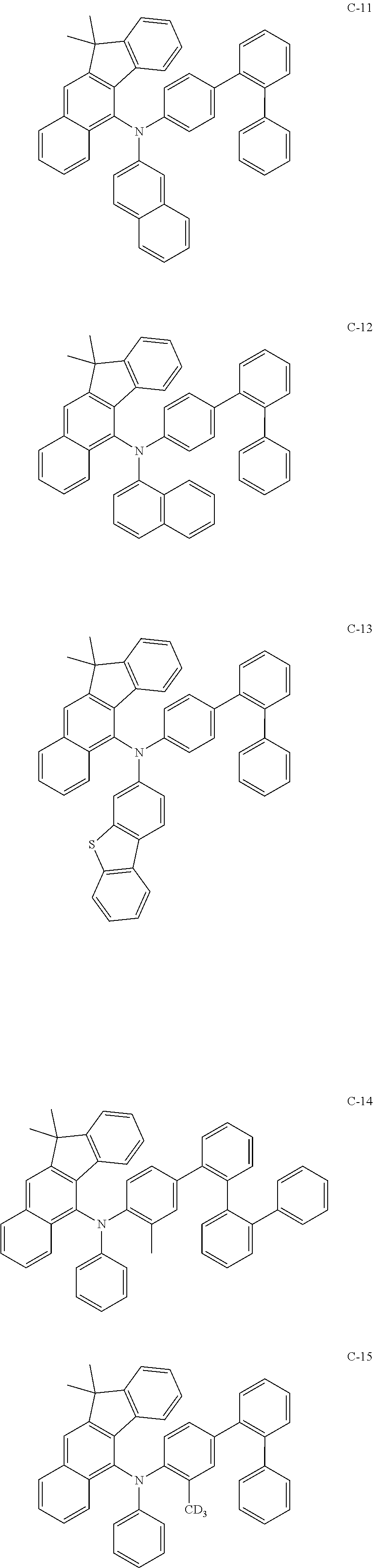 [SMILES: CC1(C)c2ccccc2-c2c1cc1ccccc1c2N(c1ccc(-c2ccccc2-c2ccccc2)cc1)c1ccc2ccccc2c1 ; CC1(C)c2ccccc2-c2c1cc1ccccc1c2N(c1ccc(-c2ccccc2-c2ccccc2)cc1)c1cccc2ccccc12 ; CC1(C)c2ccccc2-c2c1cc1ccccc1c2N(c1ccc(-c2ccccc2-c2ccccc2)cc1)c1ccc2c(c1)sc1ccccc12 ; Cc1cc(-c2ccccc2-c2ccccc2-c2ccccc2)ccc1N(c1ccccc1)c1c2c(cc3ccccc13)C(C)(C)c1ccccc1-2 ; [2H]C([2H])([2H])c1cc(-c2ccccc2-c2ccccc2)ccc1N(c1ccccc1)c1c2c(cc3ccccc13)C(C)(C)c1ccccc1-2] 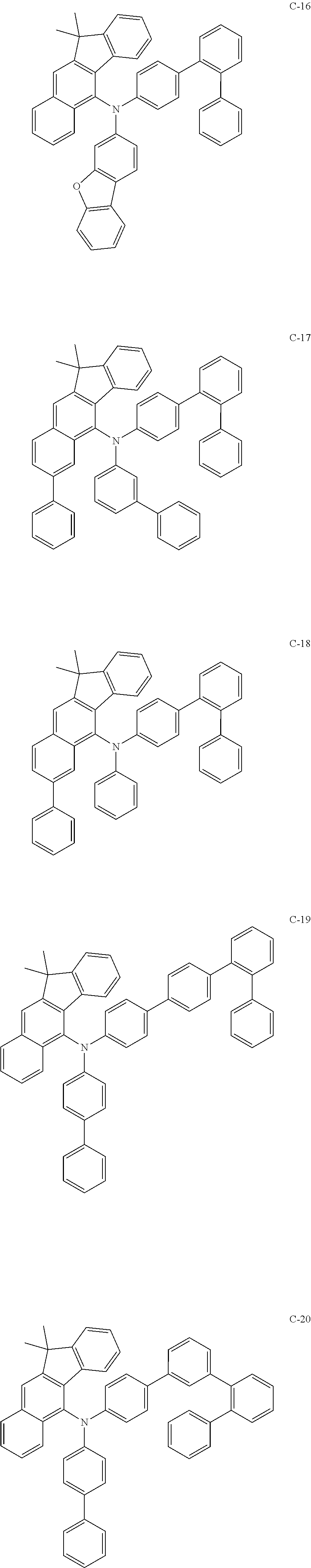 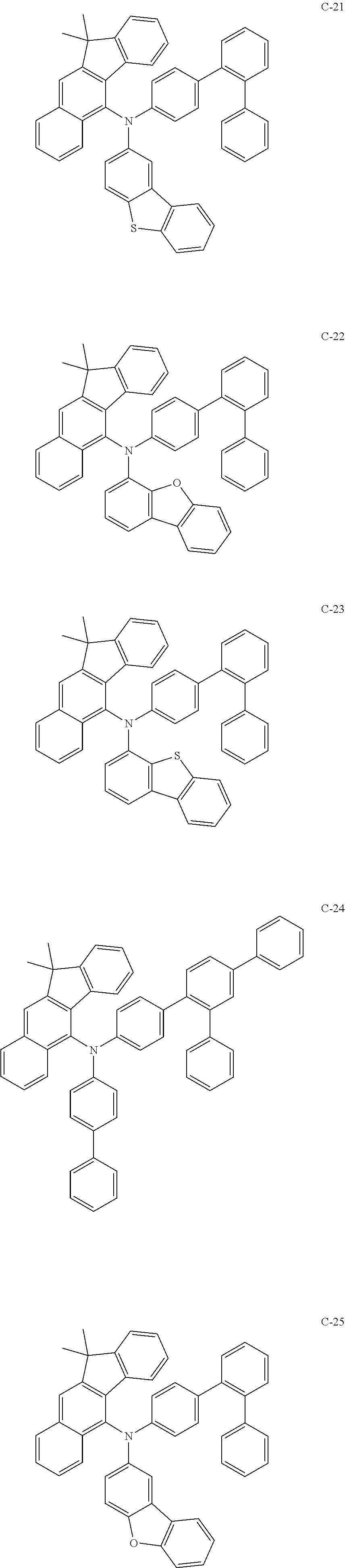 [SMILES: CC1(C)c2ccccc2-c2c1cc1ccccc1c2N(c1ccc(-c2ccccc2-c2ccccc2)cc1)c1ccc2sc3ccccc3c2c1 ; CC1(C)c2ccccc2-c2c1cc1ccccc1c2N(c1ccc(-c2ccccc2-c2ccccc2)cc1)c1cccc2c1oc1ccccc12 ; CC1(C)c2ccccc2-c2c1cc1ccccc1c2N(c1ccc(-c2ccccc2-c2ccccc2)cc1)c1cccc2c1sc1ccccc12 ; CC1(C)c2ccccc2-c2c1cc1ccccc1c2N(c1ccc(-c2ccccc2)cc1)c1ccc(-c2ccc(-c3ccccc3)cc2-c2ccccc2)cc1 ; CC1(C)c2ccccc2-c2c1cc1ccccc1c2N(c1ccc(-c2ccccc2-c2ccccc2)cc1)c1ccc2oc3ccccc3c2c1] 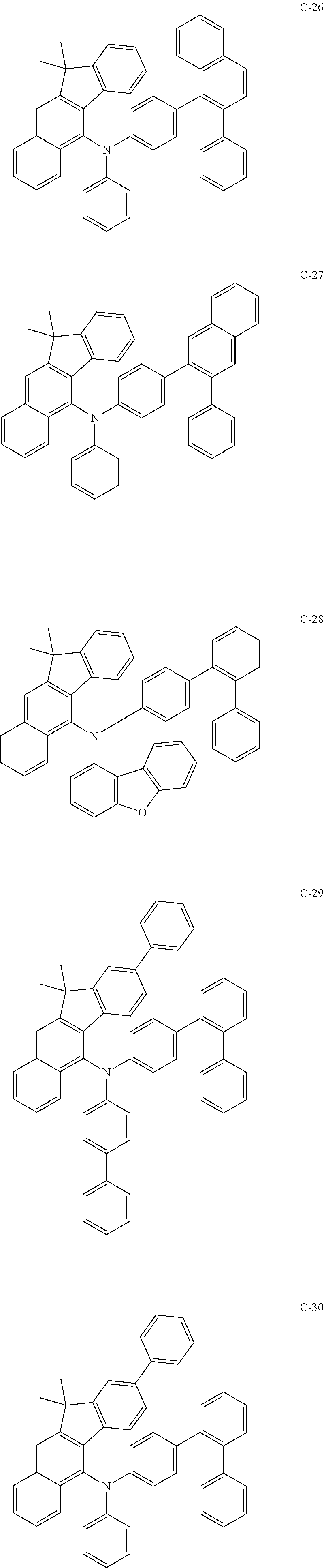 [SMILES: CC1(C)c2ccccc2-c2c1cc1ccccc1c2N(c1ccccc1)c1ccc(-c2c(-c3ccccc3)ccc3ccccc23)cc1 ; CC1(C)c2ccccc2-c2c1cc1ccccc1c2N(c1ccccc1)c1ccc(-c2cc3ccccc3cc2-c2ccccc2)cc1 ; CC1(C)c2ccccc2-c2c1cc1ccccc1c2N(c1ccc(-c2ccccc2-c2ccccc2)cc1)c1cccc2oc3ccccc3c12 ; CC1(C)c2cc(-c3ccccc3)ccc2-c2c1cc1ccccc1c2N(c1ccc(-c2ccccc2)cc1)c1ccc(-c2ccccc2-c2ccccc2)cc1 ; CC1(C)c2cc(-c3ccccc3)ccc2-c2c1cc1ccccc1c2N(c1ccccc1)c1ccc(-c2ccccc2-c2ccccc2)cc1] 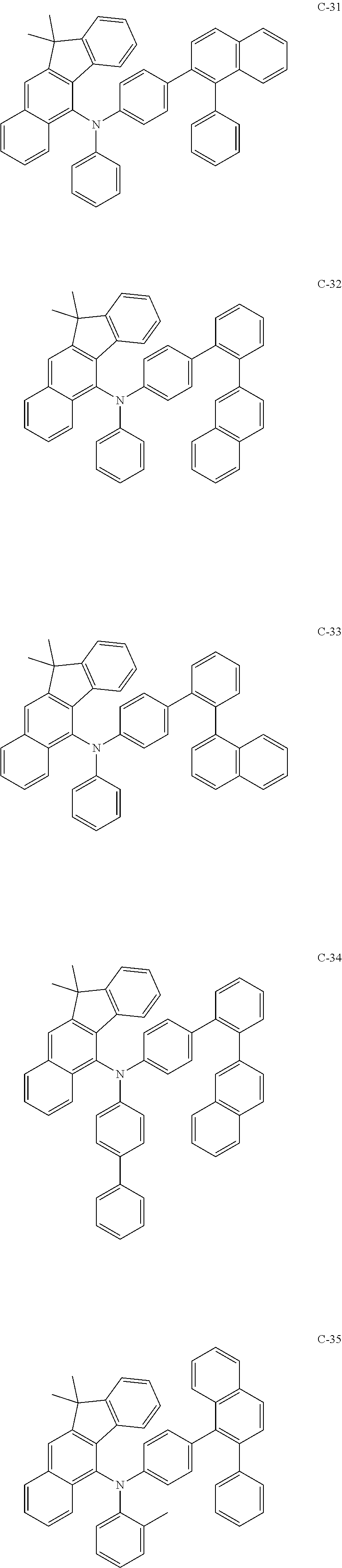 [SMILES: CC1(C)c2ccccc2-c2c1cc1ccccc1c2N(c1ccccc1)c1ccc(-c2ccc3ccccc3c2-c2ccccc2)cc1 ; CC1(C)c2ccccc2-c2c1cc1ccccc1c2N(c1ccccc1)c1ccc(-c2ccccc2-c2ccc3ccccc3c2)cc1 ; CC1(C)c2ccccc2-c2c1cc1ccccc1c2N(c1ccccc1)c1ccc(-c2ccccc2-c2cccc3ccccc23)cc1 ; CC1(C)c2ccccc2-c2c1cc1ccccc1c2N(c1ccc(-c2ccccc2)cc1)c1ccc(-c2ccccc2-c2ccc3ccccc3c2)cc1 ; Cc1ccccc1N(c1ccc(-c2c(-c3ccccc3)ccc3ccccc23)cc1)c1c2c(cc3ccccc13)C(C)(C)c1ccccc1-2] 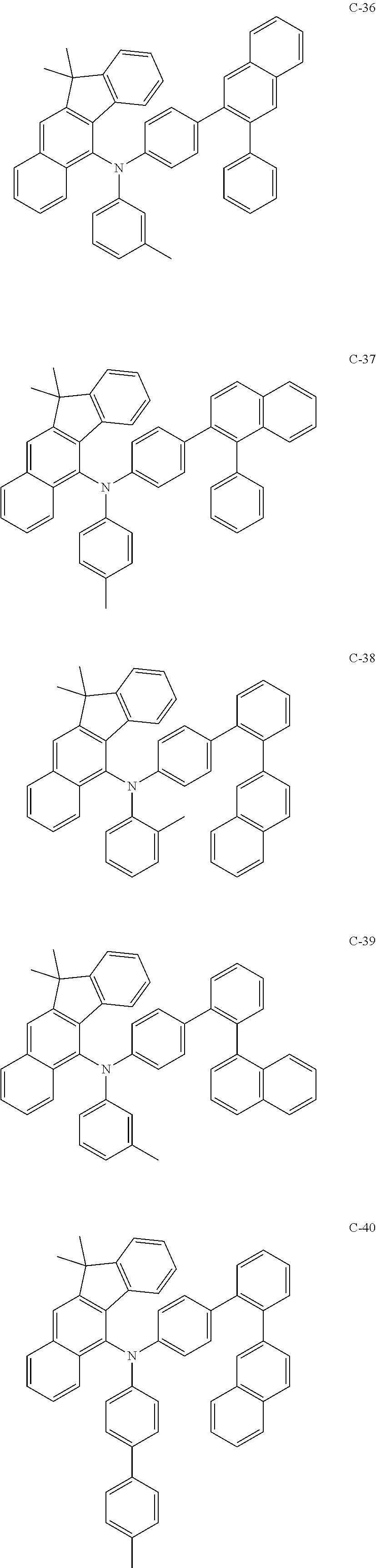 [SMILES: Cc1cccc(N(c2ccc(-c3cc4ccccc4cc3-c3ccccc3)cc2)c2c3c(cc4ccccc24)C(C)(C)c2ccccc2-3)c1 ; Cc1ccc(N(c2ccc(-c3ccc4ccccc4c3-c3ccccc3)cc2)c2c3c(cc4ccccc24)C(C)(C)c2ccccc2-3)cc1 ; Cc1ccccc1N(c1ccc(-c2ccccc2-c2ccc3ccccc3c2)cc1)c1c2c(cc3ccccc13)C(C)(C)c1ccccc1-2 ; Cc1cccc(N(c2ccc(-c3ccccc3-c3cccc4ccccc34)cc2)c2c3c(cc4ccccc24)C(C)(C)c2ccccc2-3)c1 ; Cc1ccc(-c2ccc(N(c3ccc(-c4ccccc4-c4ccc5ccccc5c4)cc3)c3c4c(cc5ccccc35)C(C)(C)c3ccccc3-4)cc2)cc1] 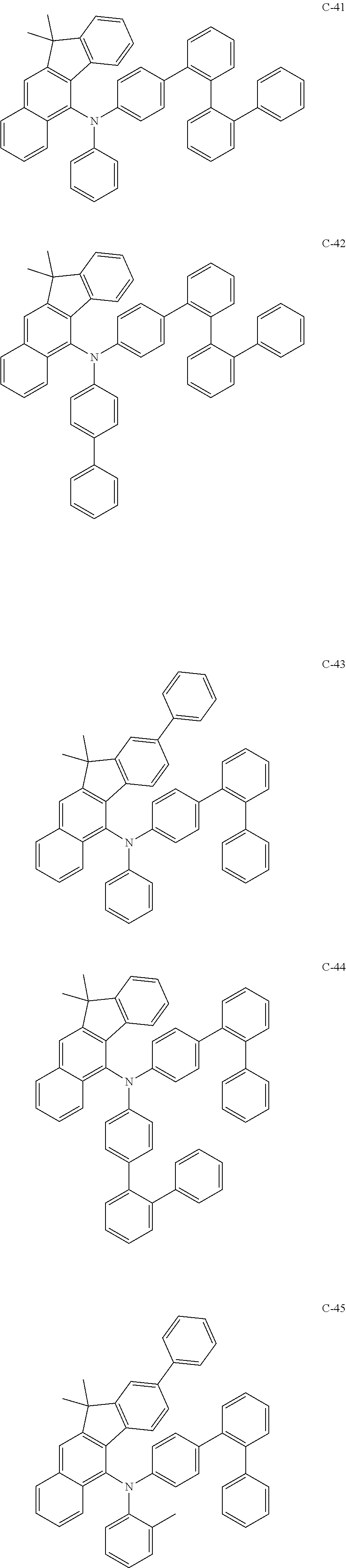 [SMILES: CC1(C)c2ccccc2-c2c1cc1ccccc1c2N(c1ccccc1)c1ccc(-c2ccccc2-c2ccccc2-c2ccccc2)cc1 ; CC1(C)c2ccccc2-c2c1cc1ccccc1c2N(c1ccc(-c2ccccc2)cc1)c1ccc(-c2ccccc2-c2ccccc2-c2ccccc2)cc1 ; CC1(C)c2cc(-c3ccccc3)ccc2-c2c1cc1ccccc1c2N(c1ccccc1)c1ccc(-c2ccccc2-c2ccccc2)cc1 ; CC1(C)c2ccccc2-c2c1cc1ccccc1c2N(c1ccc(-c2ccccc2-c2ccccc2)cc1)c1ccc(-c2ccccc2-c2ccccc2)cc1 ; Cc1ccccc1N(c1ccc(-c2ccccc2-c2ccccc2)cc1)c1c2c(cc3ccccc13)C(C)(C)c1cc(-c3ccccc3)ccc1-2] 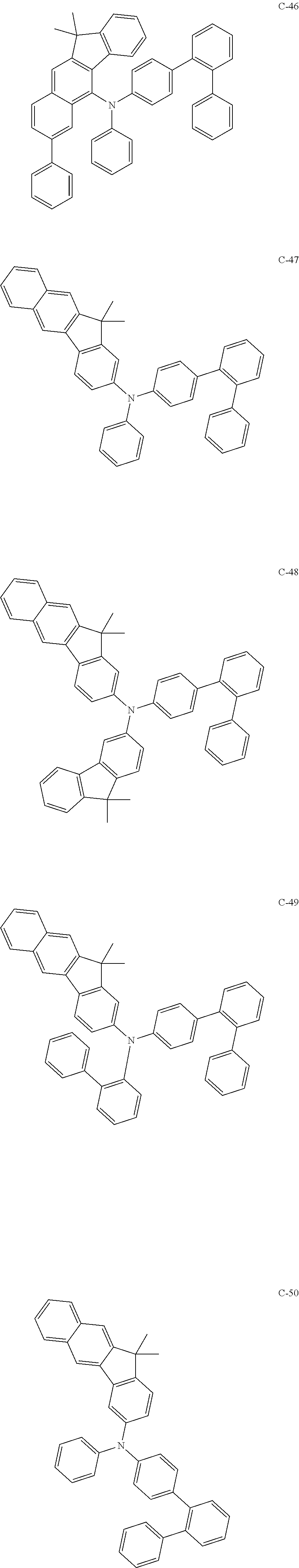 [SMILES: CC1(C)c2ccccc2-c2c1cc1ccc(-c3ccccc3)cc1c2N(c1ccccc1)c1ccc(-c2ccccc2-c2ccccc2)cc1 ; CC1(C)c2cc(N(c3ccccc3)c3ccc(-c4ccccc4-c4ccccc4)cc3)ccc2-c2cc3ccccc3cc21 ; CC1(C)c2ccccc2-c2cc(N(c3ccc(-c4ccccc4-c4ccccc4)cc3)c3ccc4c(c3)C(C)(C)c3cc5ccccc5cc3-4)ccc21 ; CC1(C)c2cc(N(c3ccc(-c4ccccc4-c4ccccc4)cc3)c3ccccc3-c3ccccc3)ccc2-c2cc3ccccc3cc21 ; CC1(C)c2ccc(N(c3ccccc3)c3ccc(-c4ccccc4-c4ccccc4)cc3)cc2-c2cc3ccccc3cc21] 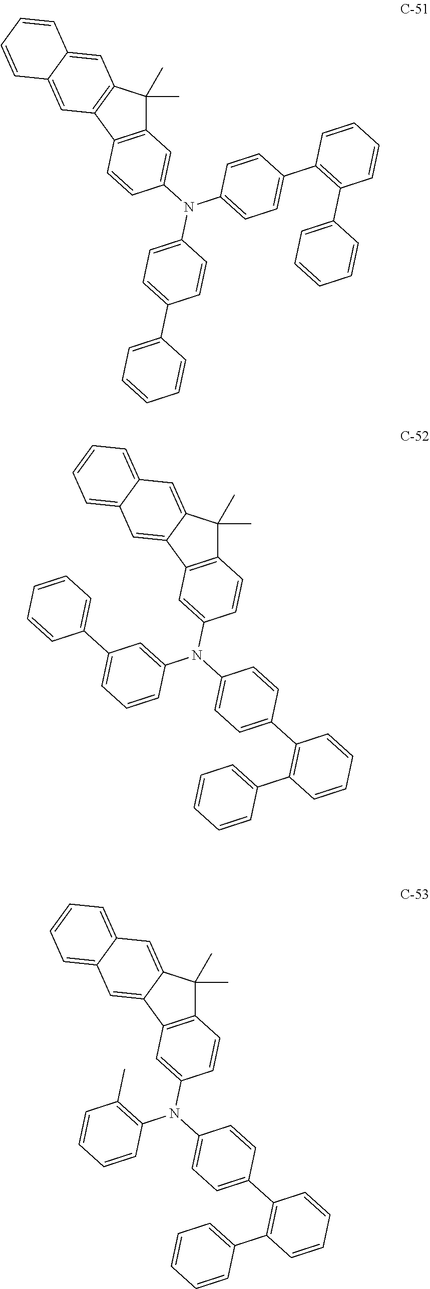 [SMILES: CC1(C)c2cc(N(c3ccc(-c4ccccc4)cc3)c3ccc(-c4ccccc4-c4ccccc4)cc3)ccc2-c2cc3ccccc3cc21 ; CC1(C)c2ccc(N(c3ccc(-c4ccccc4-c4ccccc4)cc3)c3cccc(-c4ccccc4)c3)cc2-c2cc3ccccc3cc21 ; Cc1ccccc1N(c1ccc(-c2ccccc2-c2ccccc2)cc1)c1ccc2c(c1)-c1cc3ccccc3cc1C2(C)C] 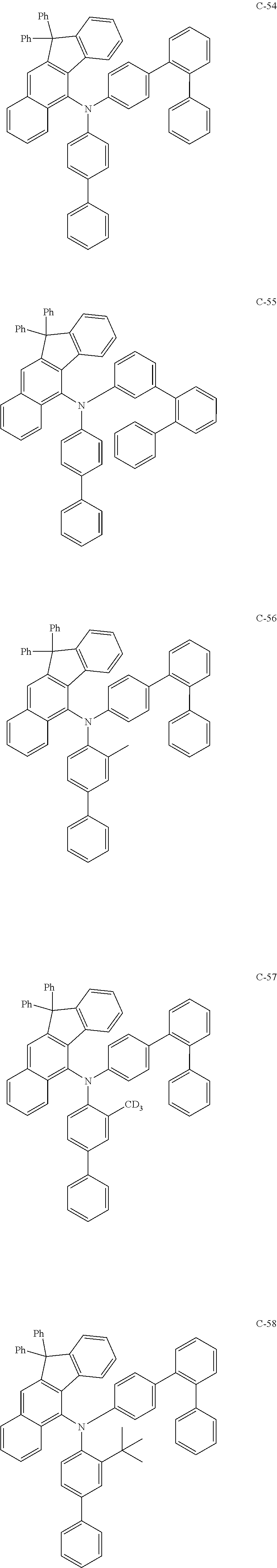 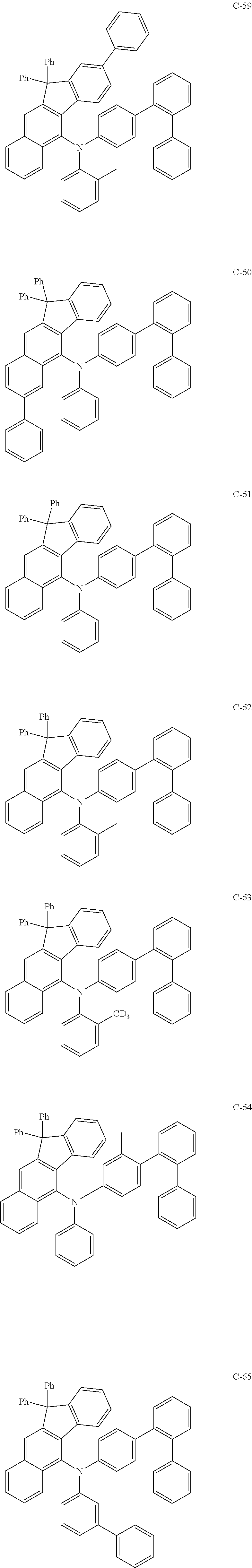 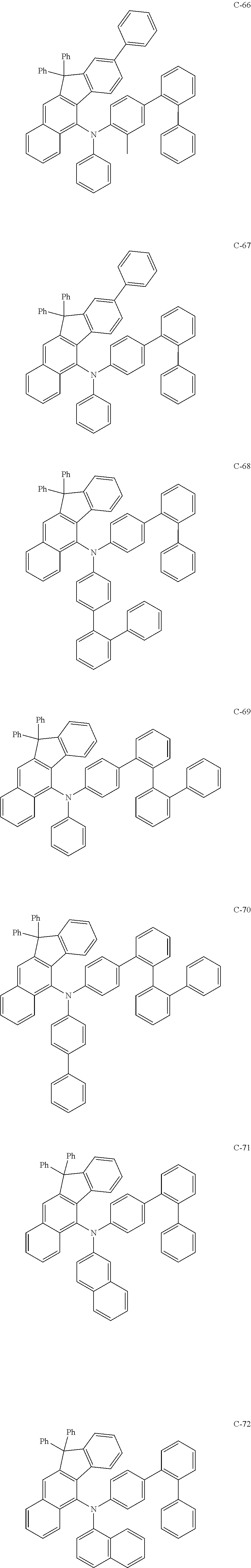 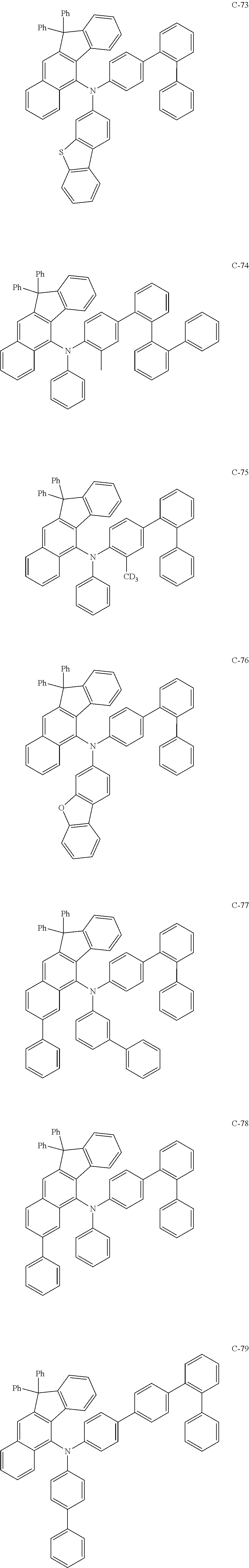 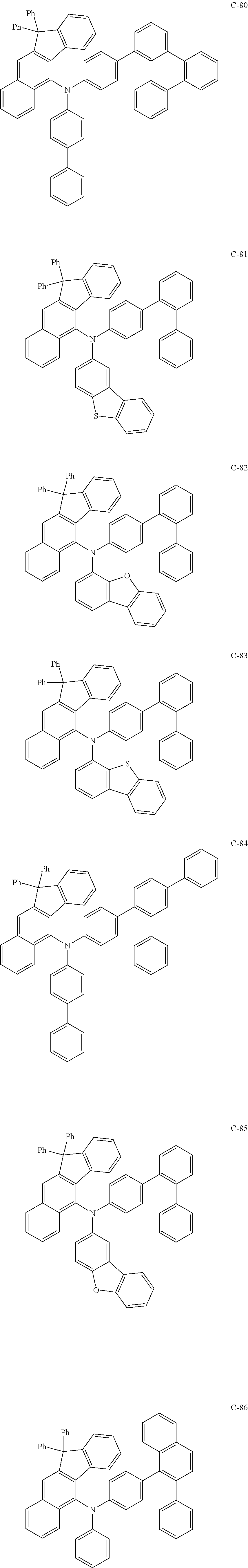 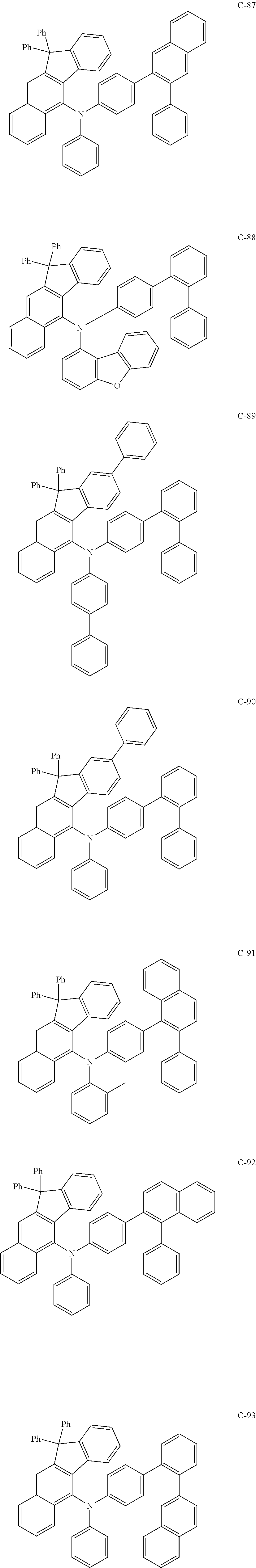 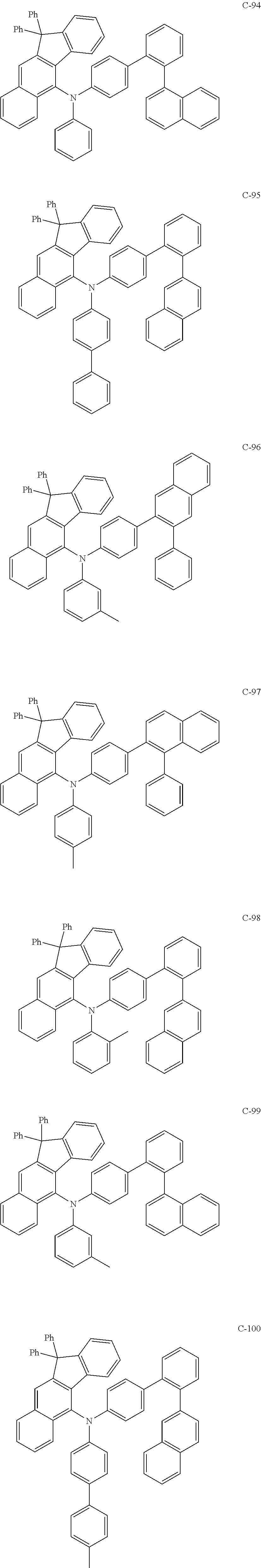 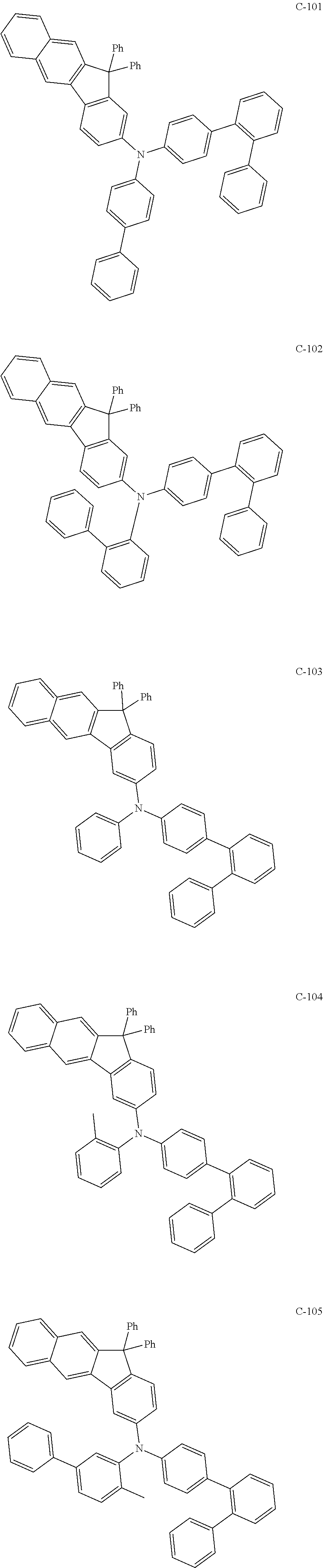 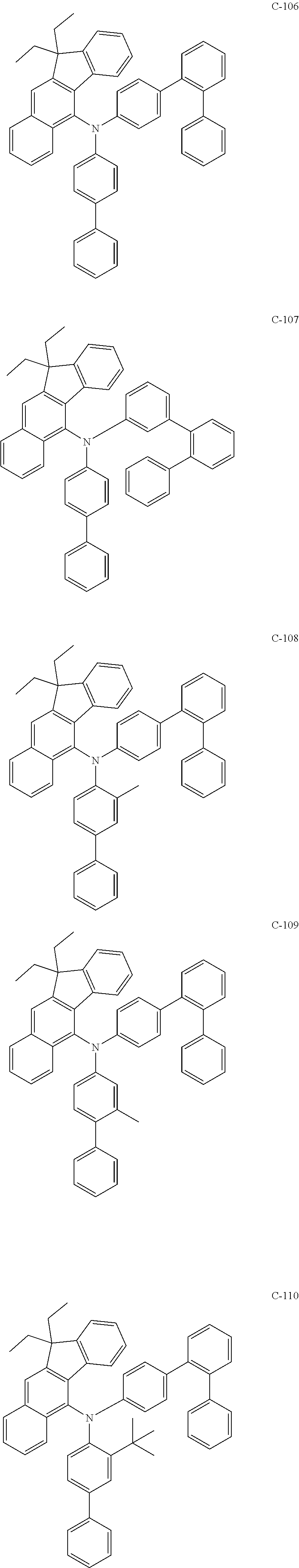 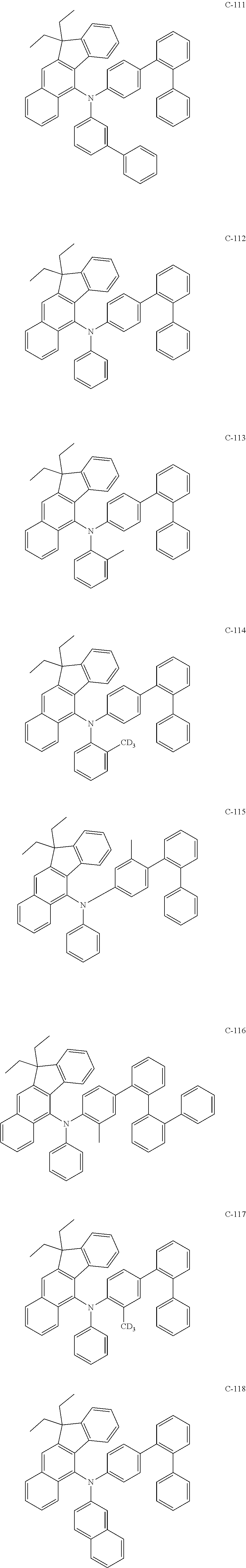 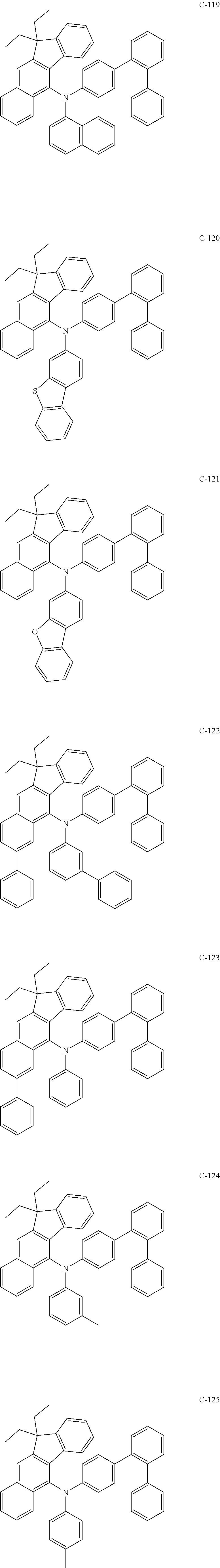 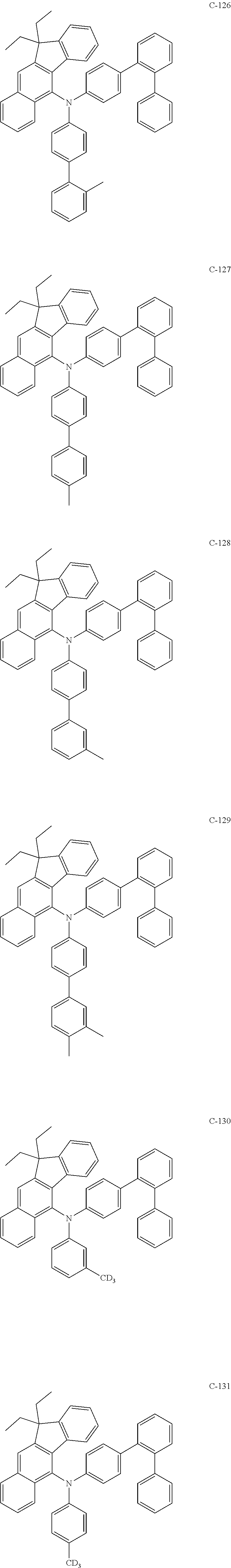 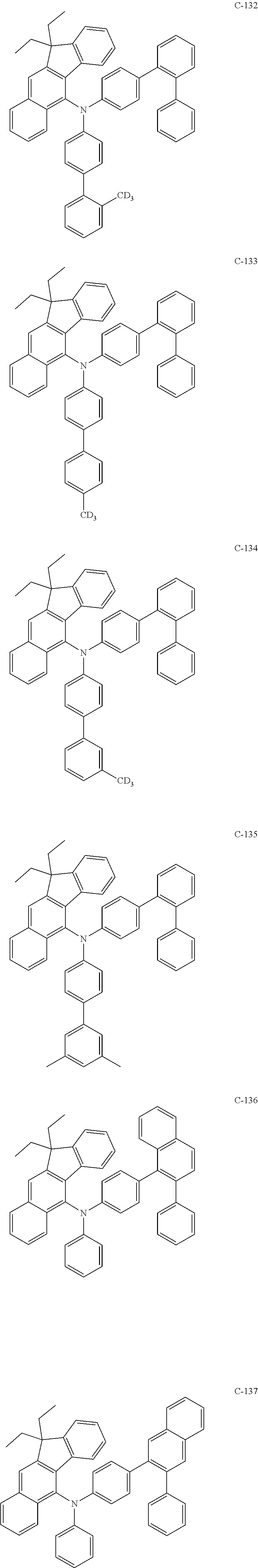 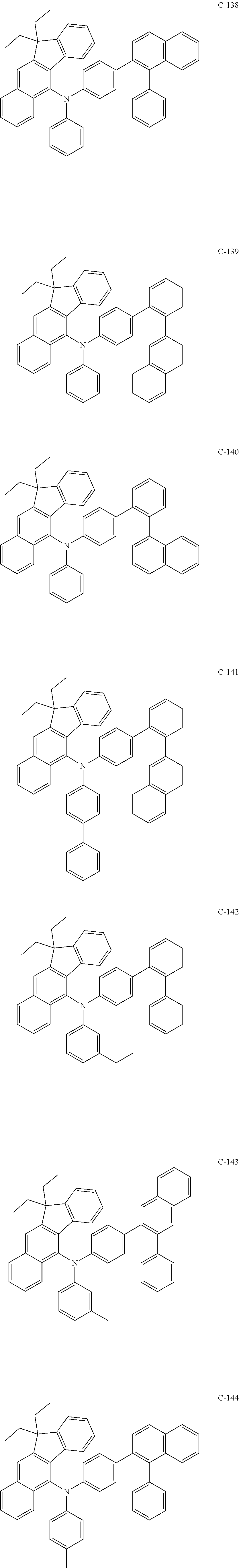 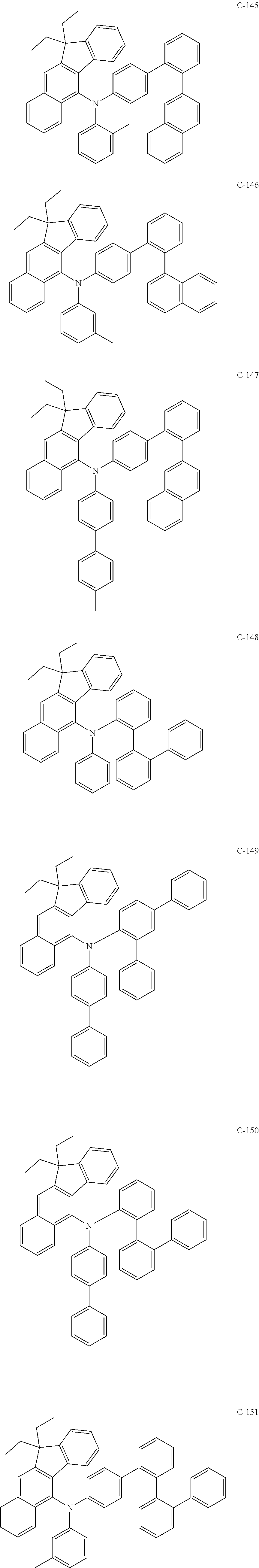 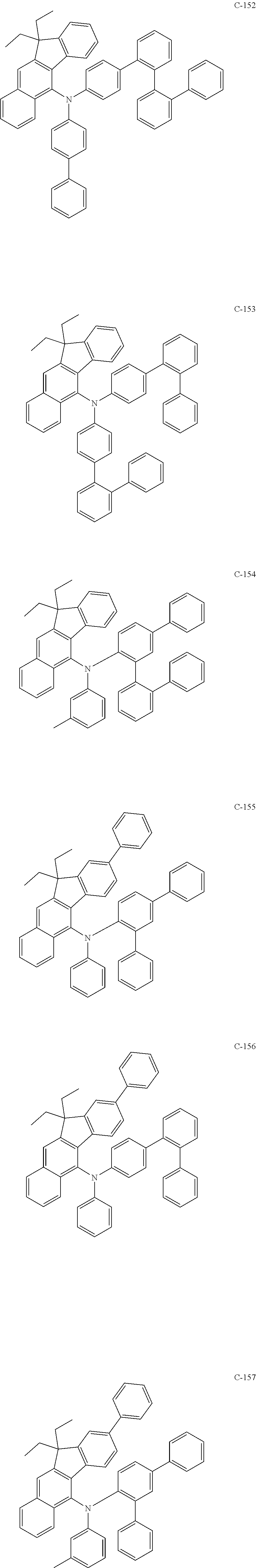 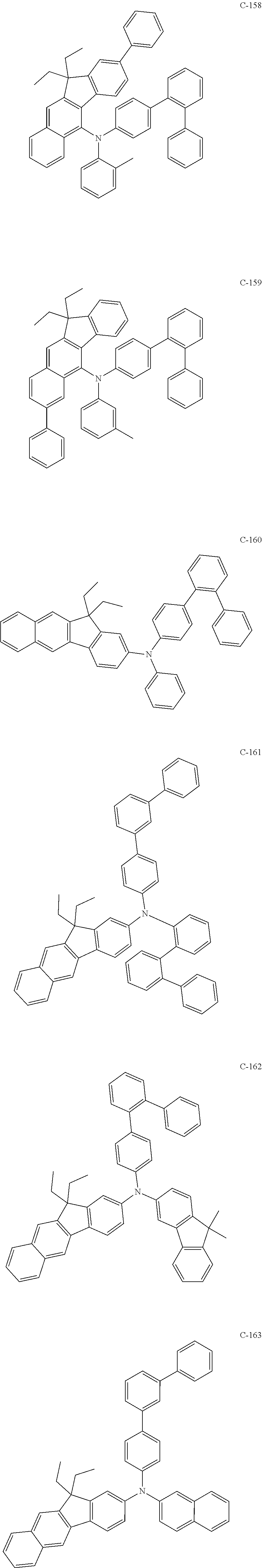 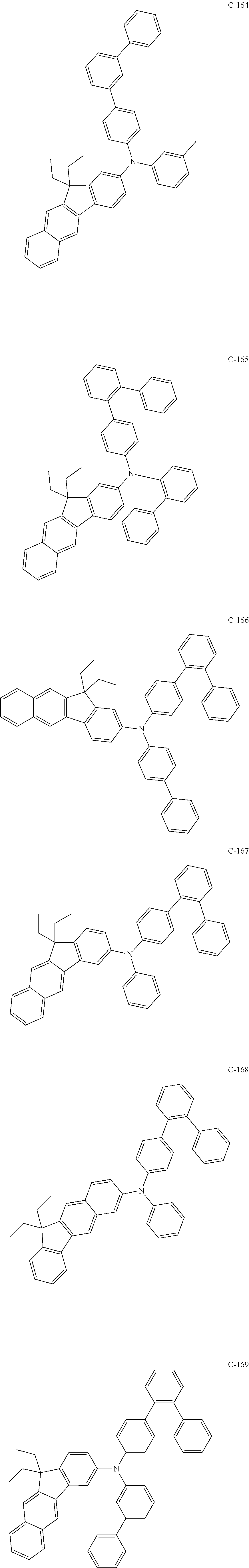 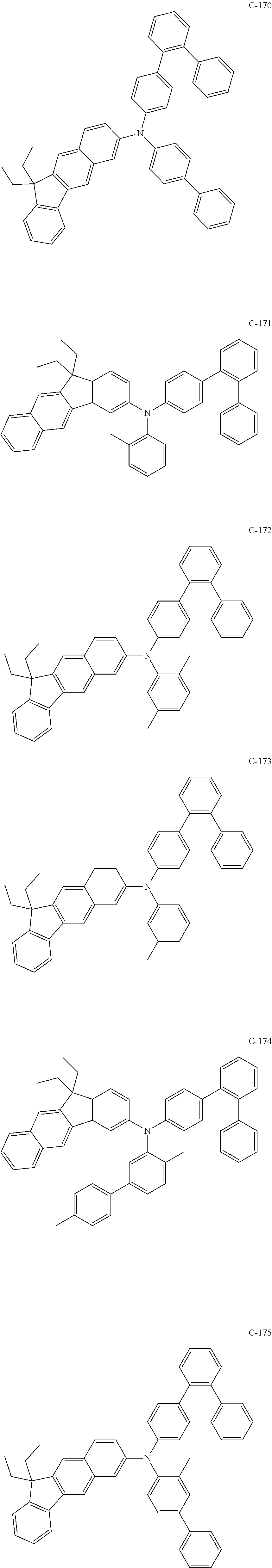 [SMILES: CCC1(CC)c2ccccc2-c2cc3cc(N(c4ccc(-c5ccccc5)cc4)c4ccc(-c5ccccc5-c5ccccc5)cc4)ccc3cc21 ; CCC1(CC)c2ccc(N(c3ccc(-c4ccccc4-c4ccccc4)cc3)c3ccccc3C)cc2-c2cc3ccccc3cc21 ; CCC1(CC)c2ccccc2-c2cc3cc(N(c4ccc(-c5ccccc5-c5ccccc5)cc4)c4cc(C)ccc4C)ccc3cc21 ; CCC1(CC)c2ccccc2-c2cc3cc(N(c4ccc(-c5ccccc5-c5ccccc5)cc4)c4cccc(C)c4)ccc3cc21 ; CCC1(CC)c2ccc(N(c3ccc(-c4ccccc4-c4ccccc4)cc3)c3cc(-c4ccc(C)cc4)ccc3C)cc2-c2cc3ccccc3cc21 ; CCC1(CC)c2ccccc2-c2cc3cc(N(c4ccc(-c5ccccc5-c5ccccc5)cc4)c4ccc(-c5ccccc5)cc4C)ccc3cc21] 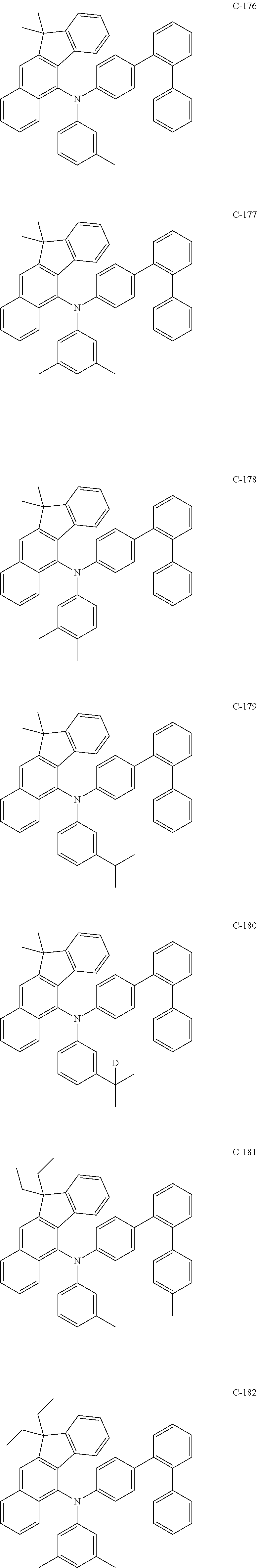 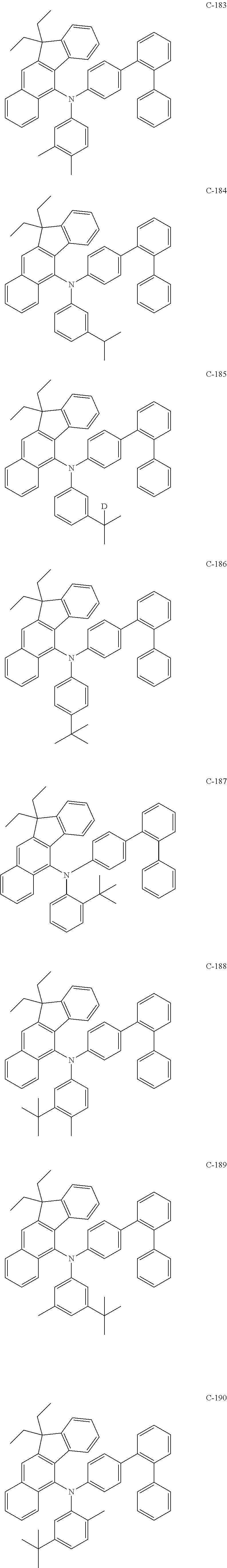 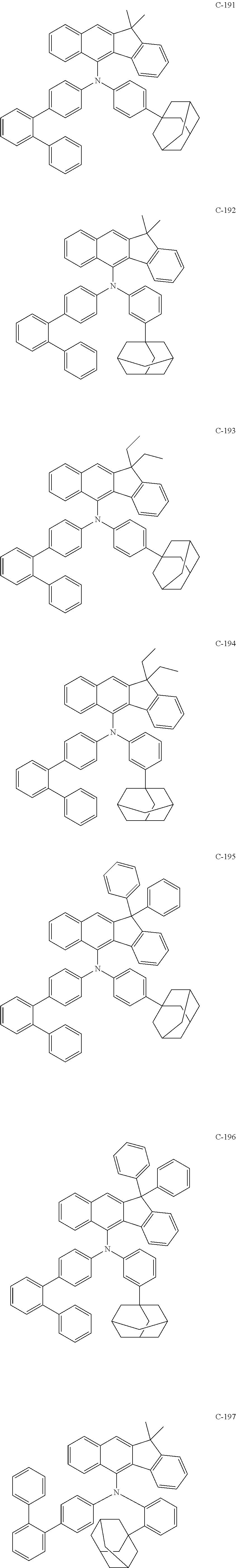 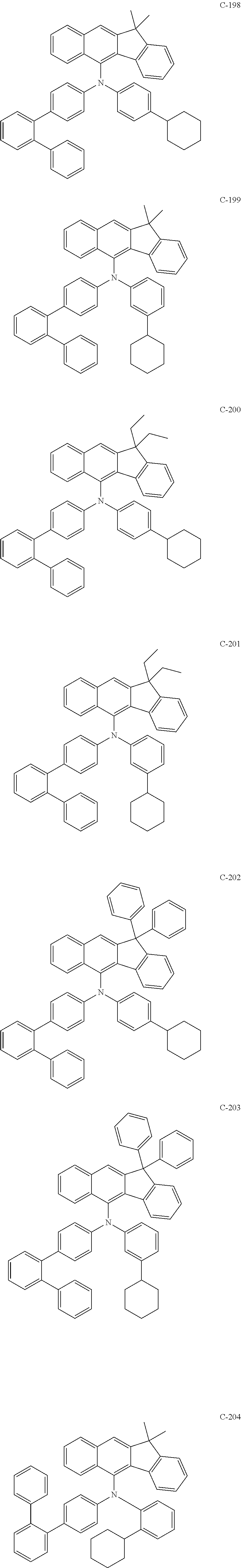 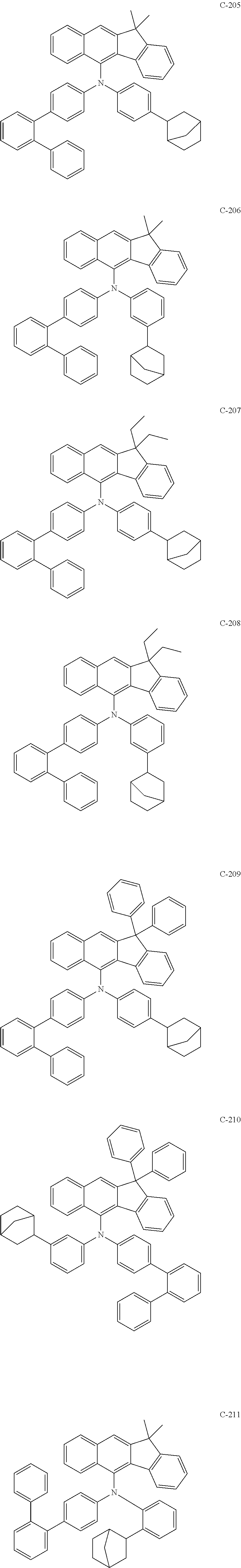
in the structures, Ph represents a phenyl group.

9. An organic electroluminescent material comprising the organic electroluminescent compound according to claim 1.

10. An organic electroluminescent device comprising the organic electroluminescent compound according to claim 1.

11. The organic electroluminescent device according to claim 10, wherein the organic electroluminescent compound is comprised in at least one layer of a light-emitting layer, a first hole transport layer, a second hole transport layer, a hole auxiliary layer, an electron blocking layer, and a light-emitting auxiliary layer.
